(19) United States
(12) Patent Application Publication
(10) Pub. No.: US 2004/0018194 A1
(43) Pub. Date: Jan. 29, 2004

(54) Recombinant anti-CD30 antibodies and uses thereof

(76) Inventors: Joseph A. Francisco, Edmonds, WA (US); Grant Risdon, Seattle, WA (US); Alan F. Wahl, Mercer Island, WA (US); Clay Siegall, Edmonds, WA (US); Peter D. Senter, Seattle, WA (US); Sveltana Doronina, Snohomish, WA (US); Brian E. Toki, Lynnwood, WA (US)

Correspondence Address: Pennie & Edmonds LLP, 1155 Avenue of the Americas, New York, NY, 10036-2711, US

(21) Appl. No.: 10/447,257

(22) Filed: May 28, 2003

Related U.S. Application Data
(63) Continuation-in-part of application No. PCT/US01/44811, filed on Nov. 28, 2001.
Application No. PCT/US01/44811 is a continuation-in-part of application No. 09/724,406, filed on Nov. 28, 2000.
(60) Provisional application No. 60/400,403, filed on Jul. 31, 2002.

(51) Int. Cl. A61K 39/395; C07K 16/30

(52) U.S. Cl. 424/144.1; 530/388.22

(57) ABSTRACT

The present invention relates to methods and compositions for the treatment of Hodgkin's Disease, comprising administering proteins characterized by their ability to bind to CD30, or compete with monoclonal antibodies AC10 or HeFi-1 for binding to CD30, and exert a cytostatic or cytotoxic effect on Hodgkin's disease cells in the absence of effector cells or complement. Such proteins include derivatives of monoclonal antibodies AC10 and HeFi-1. The proteins of the invention can be human, humanized, or chimeric antibodies; further, they can be conjugated to cytotoxic agents such as chemotherapeutic drugs. The invention further relates to nucleic acids encoding the proteins of the invention. The invention yet further relates to a method for identifying an anti-CD30 antibody useful for the treatment or prevention of Hodgkin's Disease.

This application is a continuation-in-part of copending International Application No. PCT/US01/44811, filed Nov. 28, 2001, which is a continuation-in-part of U.S. application Ser. No. 09/724,406, filed Nov. 28, 2000, each of which is incorporated by reference herein in its entirety.

### 1. FIELD OF THE INVENTION

The present invention relates to methods and compositions for the treatment of Hodgkin's Disease, comprising administering a protein that binds to CD30. Such proteins include recombinant/variant forms of monoclonal antibodies AC10 and HeFi-1, and derivatives thereof. This invention relates to a novel class of monoclonal antibodies directed against the CD30 receptor which, in unmodified form and in the absence of effector cells and in a complement-independent manner, are capable of inhibiting the growth of CD30-expressing Hodgkin's Disease cells.

### 2. BACKGROUND OF THE INVENTION

Curative chemotherapy regimens for Hodgkin's disease represent one of the major breakthroughs in clinical oncology. Multi-agent chemotherapy regimens have increased the cure rate to more than 80% for these patients. Nevertheless, 3% of patients die from treatment-related causes, and for patients who do not respond to standard therapy or relapse after first-line treatment, the only available treatment modality is high-dose chemotherapy in combination with stem cell transplantation. This treatment is associated with an 80% incidence of mortality, significant morbidity and a five-year survival rate of less than 50% (See e.g., Engert, et al., 1999, Seminars in Hematology 36:282-289).

The primary cause for tumor relapse is the development of tumor cell clones resistant to the chemotherapeutic agents. Immunotherapy represents an alterative strategy which can potentially bypass resistance. Monoclonal antibodies for specific targeting of malignant tumor cells has been the focus of a number of immunotherapeutic approaches. For several malignancies, antibody-based therapeutics are now an acknowledged part of the standard therapy. The engineered anti-CD20 antibody Rituxan®, for example, was approved in late 1997 for the treatment of relapsed low-grade NHL.

CD30 is a 120 kilodalton membrane glycoprotein (Froese et al., 1987, J. Immunol. 139: 2081-87) and a member of the TNF-receptor superfamily. This family includes TNF-RI, TNF-RII, CD30, CD40, OX-40 and RANK, among others.

CD30 is a proven marker of malignant cells in Hodgkin's disease (HD) and anaplastic large cell lymphoma (ALCL), a subset of non-Hodgkin's (NHL) lymphomas (Dürkop et al., 1992, Cell 88:421-427). Originally identified on cultured Hodgkin's-Reed Steinberg (H-RS) cells using the monoclonal antibody Ki-1 (Schwab et al., 1982, Nature 299:65-67), CD30 is highly expressed on the cell surface of all HD lymphomas and the majority of ALCL, yet has very limited expression in normal tissues to small numbers of lymphoid cells in the perifollicular areas (Josimovic-Alasevic et al., 1989, Eur. J. Immunol. 19:157-162). Monoclonal antibodies specific for the CD30 antigen have been explored as vehicles for the delivery of cytostatic drugs, plant toxins and radioisotopes in both pre-clinical models and clinical studies (Engert et al., 1990, Cancer Research 50:84-88; Barth et al., 2000, Blood 95:3909-3914). In patients with HD, targeting of the CD30 antigen could be achieved with low doses of the anti-CD30 mAb, BerH2 (Falini et al., 1992, British Journal of Haematology 82:38-45). Yet, despite successful in vivo targeting of the malignant tumor cells, none of the patients experienced tumor regression. In a subsequent clinical trial, a toxin (saporin) was chemically conjugated to the antibody BerH2 and all four patients demonstrated rapid and substantial reductions in tumor mass (Falini et al., 1992, Lancet 339:1195-1196).

These observations underscore the validity of the CD30 receptor as a target antigen. However, all of the patients treated with the mAb-toxin conjugate developed antibodies to the toxin. One of the major limitations of immunotoxins is their inherent immunogenicity that results in the development of antibodies to the toxin molecule and neutralizes their effects (Tsutsumi et al., 2000, Proc. Nat'l Acad. Sci. U.S.A. 97:8545-8553). Additionally, the liver toxicity and vascular leak syndrome associated with immunotoxins potentially limits the ability to deliver curative doses of these agents (Tsutsumi et al., 2000, Proc. Nat'l Acad. Sci. U.S.A. 97:8545-8553).

2.1 CD30 Monoclonal Antibodies

CD30 was originally identified by the monoclonal antibody Ki-1 and initially referred to as the Ki-1 antigen (Schwab et al., 1982, Nature 299:65-67). This mAb was developed against Hodgkin and Reed-Sternberg (H-RS) cells, the malignant cells of Hodgkin's disease (HD). A second mAb, capable of binding a formalin resistant epitope, different from that recognized by Ki-1 was subsequently described (Schwarting et al., 1989 Blood 74:1678-1689). The identification of four additional antibodies resulted in the creation of the CD30 cluster at the Third Leucocyte Typing Workshop in 1986 (McMichael, A., ed., 1987, Leukocyte Typing III (Oxford: Oxford University Press)).

2.2 CD30 Monoclonal Antibody-Based Therapeutics

The utility of CD30 mAbs in the diagnosis and staging of HD led to their evaluation as potential tools for immunotherapy. In patients with HD, specific targeting of the CD30 antigen was achieved with low doses (30-50 mg) of the anti-CD30 mAb BerH2 (Falini et al., 1992, British Journal of Haematology 82:38-45). Despite successful targeting in vivo of the malignant H-RS tumor cells, none of the patients experienced tumor regressions.

Based on these results, it was concluded that efficacy with CD30 mAb targeted immunotherapy could not be achieved with unmodified antibodies (Falini et al., 1992, Lancet 339:1195-1196). In a subsequent clinical trial, treatment of four patients with refractory HD with a toxin, saporin, chemically conjugated to the mAb BerH2 demonstrated rapid and substantial, although transient, reductions in tumor mass (Falini et al., 1992, Lancet 339:1195-1196). In recent years, investigators have worked to refine the approaches for treating CD30-expressing neoplastic cells. Examples include the development of recombinant single chain immunotoxins (Barth et al., 2000, Blood 95:3909-3914), anti-CD16-/CD30 bi-specific mAbs (Renner et al., 2000, Cancer Immunol. Immunother. 49:173-180), and the identification of new anti-CD30 mAbs which prevent the release of CD30 molecules from the cell surface (Hom-Lohrens et al., 1995, Int. J. Cancer 60:539-544). This focus has dismissed the potential of anti-CD30 mAbs with signaling activity in the treatment of Hodgkin's disease.

2.3 Identification of Anti-CD30 Monoclonal Antibodies with Agonist Activity

In cloning and characterizing the biologic activity of the human CD30 ligand (CD30L), two mAbs, M44 and M67, were described which mimicked the activity of CD30L induced receptor crosslinking (Gruss et al., 1994, Blood 83:2045-2056). In in vitro assays, these mAbs, in immobilized form, were capable of stimulating the proliferation of activated T-cells and the Hodgkin's disease cell lines of T-cell origin, L540 and HDLM-2. In contrast, these mAbs had little effect on the Hodgkin's cell lines of B-cell origin, L428 and KM-H2 (Gruss et al., 1994, Blood 83:2045-2056). In all of these assays, the binding of the CD30 receptor by the anti-CD30 mAb Ki-1 had little effect.

The proliferative activity of these agonist anti-CD30 mAbs on Hodgkin's cell lines suggested that anti-CD30 mAbs possessing signaling activity would not have any utility in the treatment of HD.

In contrast, it has recently been shown that anti-CD30 mAbs can inhibit the growth of ALCL cells, including Karpas-299, through induction of cell cycle arrest and without induction of apoptosis (Hubinger et al., 2001, Oncogene 20:590-598). Furthermore, the presence of immobilized M44 and M67 mAbs strongly inhibits the proliferation of cell lines representing CD30-expressing ALCL (Gruss et al., 1994, Blood 83:2045-2056). This inhibitory activity against ALCL cell lines was further extended to in vivo animal studies. The survival of SCID mice bearing ALCL tumor xenografts was significantly increased following the administration of the mAb M44. In addition, the anti-CD30 mAb HeFi-1, recognizing a similar epitope as that of M44, also prolonged survival in this animal model (Tian et al., 1995, Cancer Research 55:5335-5341).

2.3.1 Monoclonal Antibody AC10

The majority of murine anti-CD30 mAbs known in the art have been generated by immunization of mice with HD cell lines or purified CD30 antigen. AC10, originally termed C10 (Bowen et al., 1993, J. Immunol. 151:5896-5906), is distinct in that this anti-CD30 mAb that was prepared against a human NK-like cell line, YT (Bowen et al., 1993, J. Immunol. 151:5896-5906). Initially, the signaling activity of this mAb was evidenced by the down regulation of the cell surface expression of CD28 and CD45 molecules, the up regulation of cell surface CD25 expression and the induction of homotypic adhesion following binding of C10 to YT cells.

2.3.2 Monoclonal Antibody HeFi-1

HeFi-1 is an anti-CD30 mAb which was produced by immunizing mice with the L428 Hodgkin's disease cell line (Hecht et al., 1985, J. Immunol. 134:4231-4236). Co-culture of HeFi-1 with the Hodgkin's disease cell lines L428 or L540 failed to reveal any direct effect of the mAb on the viability of these cell lines. In vitro and in vivo antitumor activity of HeFi-1 was described by Tian et al against the Karpas 299 ALCL cell line (Tian et al., 1995, Cancer Research 55:5335-5341).

2.4 Direct Anti-Tumor Activity of Signaling CD30 Antibodies

Monoclonal antibodies represent an attractive approach to targeting specific populations of cells in vivo. Native mAbs and their derivatives may eliminate tumor cells by a number of mechanisms including, but not limited to, complement activation, antibody dependent cellular cytotoxicity (ADCC), inhibition of cell cycle progression and induction of apoptosis (Tutt et al., 1998, J. Immunol. 161:3176-3185).

As described above, mAbs to the CD30 antigen such as Ki-1 and Ber-H2 failed to demonstrate direct antitumor activity (Falini et al., 1992, British Journal of Haematology 82:38-45; Gruss et al., 1994, Blood 83:2045-2056). While some signaling mAbs to CD30, including M44, M67 and HeFi-1, have been shown to inhibit the growth of ALCL lines in vitro (Gruss et al., 1994, Blood 83:2045-2056) or in vivo (Tian et al., 1995, Cancer Res. 55:5335-5341), known anti-CD30 antibodies have not been shown to be effective in inhibiting the proliferation of HD cells in culture. In fact, two signaling anti-CD30 mAbs, M44 and M67, which inhibited the growth of the ALCL line Karpas-299, were shown to enhance the proliferation of T-cell-like HD lines in vitro while showing no effect on B-cell-like HD lines (.Gruss et al., 1994, Blood 83:2045-2056).

The conjugate of antibody Ki-1 with the Ricin A-chain made for a rather ineffective immunotoxin and it was concluded that this ineffectiveness was due to the rather low affinity of antibody Ki-1 (Engert et al., 1990, Cancer Research 50:84-88). Two other reasons may also account for the weak toxicity of Ki-1-Ricin A-chain conjugates: a) Antibody Ki-1 enhanced the release of the sCD30 from the Hodgkin-derived cell lines L428 and L540 as well as from the CD30+ non-Hodgkin's lymphoma cell line Karpas 299 (Hansen et al., 1991, Immunobiol. 183:214); b) the relatively great distance of the Ki-1 epitope from the cell membrane is also not favorable for the construction of potent immunotoxins (Press et al., 1988, J. Immunol. 141:4410-4417; May et al., 1990, J. Immunol. 144:3637-3642).

At the Fourth Workshop on Leukocyte Differentiation Antigens in Vienna in February 1989, monoclonal antibodies were submitted by three different laboratories and finally characterized as belonging to the CD30 group. Co-cultivation experiments by the inventors of L540 cells with various antibodies according to the state of the art, followed by the isolation of sCD30 from culture supernatant fluids, revealed that the release of the sCD30 was most strongly increased by antibody Ki-1, and weakly enhanced by the antibody HeFi-1, whilst being more strongly inhibited by the antibody Ber-H2. However, the antibody Ber-H2 also labels a subpopulation of plasma cells (Schwarting et al., 1988, Blood 74:1678-1689) and G. Pallesen (G. Pallesen, 1990, Histopathology 16:409-413) describes, on page 411, that Ber-H2 is cross-reacting with an epitope of an unrelated antigen which is altered by formaldehyde.

There is a need in the art for therapeutics with increased efficacy to treat or prevent Hodgkin's Disease, a need provided by the present invention. Clinical trials and numerous pre-clinical evaluations have failed to demonstrate antitumor activity of a number of anti-CD30 mAbs in unmodified form against cells representative of Hodgkin's disease. Under conditions similar to those utilized by Gruss et al. in their evaluations of mAbs Ki-1, M44 and M67 (Gruss et al., 1994, Blood 83:2045-2056), the present inventors demonstrate a class of CD30 mAbs which is functionally distinct from those previously described. This class of anti-CD30 mAbs is capable of inhibiting the in vitro growth of all Hodgkin's lines tested. Furthermore, these unmodified mAbs possess in vivo antitumor activity against HD tumor xenografts.

Citation or identification of any reference herein shall not be construed as an admission that such reference is available as prior art to the present invention.

### 3. SUMMARY OF THE INVENTION

The present invention is based on the surprising discovery of a novel activity associated with a certain class of anti-CD30 antibodies, said class comprising AC10 and HeFi-1, namely their ability to inhibit, in the absence of effector cells and in a complement-independent fashion, the growth of both T-cell-like and B-cell-like Hodgkin's Disease (HD) cells.

The invention provides proteins that compete for binding to CD30 with monoclonal antibody AC10 or HeFi-1, and exert a cytostatic or cytotoxic effect on a Hodgkin's Disease cell line. The invention further provides antibodies that immunospecifically bind CD30 and exert a cytostatic or cytotoxic effect on a Hodgkin's Disease cell line. Generally, the antibodies of the invention can exert a cytostatic or cytotoxic effect on the Hodgkin's Disease cell line in the absence of conjugation to a cytostatic or cytotoxic agent, respectively.

In preferred embodiments, the antibodies of the invention can exert a cytostatic or cytotoxic effect on a Hodgkin's Disease cell line in the absence of effector cells (e.g., natural killer cells, neutrophils) and in a complement-independent manner.

The present invention thus provides an antibody that (a) immunospecifically binds CD30, (b) exerts a cytostatic or cytotoxic effect on a Hodgkin's Disease cell line, which cytostatic or cytotoxic effect is complement-independent and achieved in the absence of: conjugation to a cytostatic or cytotoxic agent, and in the absence of effector cells, and (c) is not monoclonal antibody AC10 or HeFi-1 and does not result from cleavage of AC10 or HeFi-1 with papain or pepsin. In certain embodiments, the antibody comprises a human constant domain.

The present invention further provides an antibody that (a) competes for binding to CD30 with monoclonal antibody AC10 or HeFi-1, (b) exerts a cytostatic or cytotoxic effect on a Hodgkin's Disease cell line, which cytostatic or cytotoxic effect is not complement-dependent and is achieved in the absence of conjugation to a cytostatic or cytotoxic agent and in the absence of effector cells, and (c) is not monoclonal antibody AC10 or HeFi-1 and does not result from cleavage of AC10 or HeFi-1 with papain or pepsin. In certain embodiments, the antibody comprises a human constant domain.

The present invention yet further provides an antibody that (a) immunospecifically binds CD30; (b) exerts a cytostatic or cytotoxic effect on a Hodgkin's Disease cell line, wherein said antibody exerts the cytostatic or cytotoxic effect on the Hodgkin's Disease cell line in the absence of conjugation to a cytostatic or cytotoxic agent, respectively; and (c) is not monoclonal antibody AC10 or HeFi-1 and does not result from cleavage of AC10 or HeFi-1 with papain or pepsin, wherein the cytostatic or cytotoxic effect is exhibited upon performing a method comprising (i) immobilizing said antibody in a well, said well having a culture area of about 0.33 cm²; (ii) adding 5,000 cells of the Hodgkin's Disease cell line in the presence of only RPMI with 10% fetal bovine serum or 20% fetal bovine serum to the well; (iii) culturing the cells in presence of only said antibody and RPMI with 10% fetal bovine serum or 20% fetal bovine serum for a period of 72 hours to form a Hodgkin's Disease cell culture; (iv) exposing the Hodgkin's Disease cell culture to 0.5 μCi/well of ³H-thymidine during the final 8 hours of said 72-hour period; and (v) measuring the incorporation of ³H-thymidine into cells of the Hodgkin's Disease cell culture, wherein the antibody has a cytostatic or cytotoxic effect on the Hodgkin's Disease cell line if the cells of the Hodgkin's Disease cell culture have reduced ³H-thymidine incorporation compared to cells of the same Hodgkin's Disease cell line cultured under the same conditions but not contacted with the antibody. In certain embodiments, the antibody comprises a human constant domain.

The antibodies of the invention can be purified, for example by affinity chromatography with the CD30 antigen. In certain embodiments, the antibody is at least 50%, at least 60%, at least 70% or at least 80% pure. In other embodiments, the antibody is more than 85% pure, more than 90% pure, more than 95% pure or more than 99% pure.

The invention further provides a method for the treatment or prevention of Hodgkin's Disease in a subject comprising administering to the subject, in an amount effective for said treatment or prevention, an anti-CD30 antibody of the invention. The antibody used for treatment may be in the form of a pharmaceutical composition comprising said antibody and a pharmaceutically acceptable carrier.

Thus, in a specific embodiment, the invention provides a method for the treatment or prevention of Hodgkin's Disease in a subject comprising administering to the subject, in an amount effective for said treatment or prevention, an antibody that (a) immunospecifically binds CD30, (b) exerts a cytostatic or cytotoxic effect on a Hodgkin's Disease cell line, which cytostatic or cytotoxic effect is complement-independent and achieved in the absence of: conjugation to a cytostatic or cytotoxic agent, and in the absence of effector cells, and (c) is not monoclonal antibody AC10 or HeFi-1 and does not result from cleavage of AC10 or HeFi-1 with papain or pepsin. The antibody may be in the form of a pharmaceutical composition comprising said antibody and a pharmaceutically acceptable carrier.

In another specific embodiment, the invention provides a method for the treatment or prevention of Hodgkin's Disease in a subject comprising administering to the subject, in an amount effective for said treatment or prevention, an antibody that (a) competes for binding to CD30 with monoclonal antibody AC10 or HeFi-1, (b) exerts a cytostatic or cytotoxic effect on a Hodgkin's Disease cell line, which cytostatic or cytotoxic effect is not complement-dependent and is achieved in the absence of conjugation to a cytostatic or cytotoxic agent and in the absence of effector cells, and (c) is not monoclonal antibody AC10 or HeFi-1 and does not result from cleavage of AC10 or HeFi-1 with papain or pepsin. The antibody may be in the form of a pharmaceutical composition comprising said antibody and a pharmaceutically acceptable carrier.

In yet another specific embodiment, the invention provides a method for the treatment or prevention of Hodgkin's Disease in a subject comprising administering to the subject, in an amount effective for said treatment or prevention, an antibody that (a) immunospecifically binds CD30; (b) exerts a cytostatic or cytotoxic effect on a Hodgkin's Disease cell line, wherein said antibody exerts the cytostatic or cytotoxic effect on the Hodgkin's Disease cell line in the absence of conjugation to a cytostatic or cytotoxic agent, respectively; and (c) is not monoclonal antibody AC10 or HeFi-1 and does not result from cleavage of AC10 or HeFi-1 with papain or pepsin, wherein the cytostatic or cytotoxic effect is exhibited upon performing a method comprising (i) immobilizing said antibody in a well, said well having a culture area of about 0.33 cm², (ii) adding 5,000 cells of the Hodgkin's Disease cell line in the presence of only RPMI with 10% fetal bovine serum or 20% fetal bovine serum to the well; (iii) culturing the cells in presence of only said antibody and RPMI with 10% fetal bovine serum or 20% fetal bovine serum for a period of 72 hours to form a Hodgkin's Disease cell culture; (iv) exposing the Hodgkin's Disease cell culture to 0.5 μCi/well of ³H-thymidine during the final 8 hours of said 72-hour period; and (v) measuring the incorporation of ³H-thymidine into cells of the Hodgkin's Disease cell culture, wherein the antibody has a cytostatic or cytotoxic effect on the Hodgkin's Disease cell line if the cells of the Hodgkin's Disease cell culture have reduced ³H-thymidine incorporation compared to cells of the same Hodgkin's Disease cell line cultured under the same conditions but not contacted with the antibody. The antibody may be in the form of a pharmaceutical composition comprising said antibody and a pharmaceutically acceptable carrier.

The invention further provides a method for the treatment or prevention of Hodgkin's Disease in a subject comprising administering to the subject, in an amount effective for said treatment or prevention, an antibody that immunospecifically binds CD30 and exerts a cytostatic or cytotoxic effect on a Hodgkin's Disease cell line, wherein said antibody exerts the cytostatic or cytotoxic effect on the Hodgkin's Disease cell line in the absence of conjugation to a cytostatic or cytotoxic agent, respectively; and a pharmaceutically acceptable carrier.

The invention provides a method for the treatment or prevention of Hodgkin's Disease in a subject comprising administering to the subject an amount of a protein, which protein competes for binding to CD30 with monoclonal antibody AC10 or HeFi-1, and exerts a cytostatic or cytotoxic effect on a Hodgkin's Disease cell line, which amount is effective for the treatment or prevention of Hodgkin's Disease.

The anti-CD30 antibodies of the invention may be conjugated to a cytotoxic agent. In certain embodiments, the anti-CD30 antibody of an anti-CD30 antibody-cytotoxic agent conjugate of the invention is conjugated to the cytotoxic agent via a linker, wherein the linker is hydrolyzable at a pH of less than 5.5. In a specific embodiment the linker is hydrolyzable at a pH of less than 5.0.

In certain embodiments, the anti-CD30 antibody of an anti-CD30 antibody-cytotoxic agent conjugate of the invention is conjugated to the cytotoxic agent via a linker, wherein the linker is cleavable by a protease. In a specific embodiment, the protease is a lysosomal protease. In other specific embodiments, the protease is, inter alia, a membrane-associated protease, an intracellular protease, or an endosomal protease.

In certain embodiments, the anti-CD30 antibody-cytotoxic agent conjugate of the invention is anti-CD30-valine-citrulline-MMAE (anti-CD30-val-citMMAE or anti-CD30-vcMMAE) or anti-CD30-valine-citrulline-AEFP (anti-CD30-val-citAEFP or anti-CD30-vcAEFP). In specific embodiments, the anti-CD30 antibody-cytotoxic agent conjugate of the invention is AC10-valine-citrulline-MMAE (AC10-val-citMMAE or AC10-vcMMAE) or AC10-valine-citrulline-AEFP (AC10-val-citAEFP or AC10-vcAEFP).

In certain specific embodiments, the anti-CD30 antibody-cytotoxic agent conjugate of the invention is anti-CD30-phenylalanine-lysine-MMAE (anti-CD30-phe-lysMMAE or anti-CD30-fkMMAE) or anti-CD30-phenylalanine-lysine-AEFP (anti-CD30-phe-lysAEFP or anti-CD30-fkAEFP). In specific embodiments, the anti-CD30 antibody-cytotoxic agent conjugate of the invention is AC10-phenylalanine-lysine-MMAE (AC10-phe-lysMMAE or AC10-fkMMAE) or AC10-phenylalanine-lysine-AEFP (AC10-phe-lysAEFP or AC10-fkAEFP).

The AC10 antibody in the foregoing conjugates is preferably a chimeric AC10 (cAC10) or humanized AC10 (hAC10 ) antibody. Thus, in specific embodiments, the present invention provides the following conjugates: hAC10-valine-citrulline-MMAE (hAC10-val-citMMAE or hAC10-vcMMAE), cAC10-valine-citrulline-MMAE (cAC10-val-citMMAE or cAC10-vcMMAE), hAC10-valine-citrulline-AEFP (hAC10-val-citAEFP or hAC10-vcAEFP) or cAC10-valine-citrulline-AEFP (cAC10-val-citAEFP or cAC10-vcAEFP). In other specific embodiments, the invention provides the following conjugates: hAC10-phenylalanine-lysine-MMAE (hAC10-phe-lysMMAE or hAC10-fkMMAE), cAC10-phenylalanine-lysine-MMAE (cAC10-phe-lysMMAE or cAC10-fkMMAE), hAC10-phenylalanine-lysine-AEFP (hAC10-phe-lysAEFP or hAC10-fkAEFP), or cAC10-phenylalanine-lysine-AEFP (cAC10-phe-lysAEFP or cAC10-fkAEFP).

The present invention encompasses anti-CD30 antibodies that are fusion proteins comprising the amino acid sequence of a second protein such as bryodin or a pro-drug converting enzyme.

The anti-CD30 antibodies of the invention, including conjugates and fusion proteins, can be used in conjunction with radiation therapy, chemotherapy, hormonal therapy and/or immunotherapy. In specific embodiments, the chemotherapeutic agent is a cytostatic, cytotoxic, and/or immunosuppressive agent.

In certain specific embodiments, the immunosuppressive agent is gancyclovir, acyclovir, etanercept, rapamycin, cyclosporine or tacrolimus. In other embodiments, the immunosuppressive agent is an antimetabolite, a purine antagonist (e.g., azathioprine or mycophenolate mofetil), a dihydrofolate reductase inhibitor (e.g., methotrexate), a glucocorticoid. (e.g., cortisol or aldosterone), or a glucocorticoid analogue (e.g., prednisone or dexamethasone). In yet other embodiments, the immunosuppressive agent is an alkylating agent (e.g., cyclophosphamide). In yet other embodiments, the immunosuppressive agent is an anti-inflammatory agent, including but not limited to a cyclooxygenase inhibitor, a 5-lipoxygenase inhibitor, and a leukotriene receptor antagonist.

The present invention further provides an antibody that (i) immunospecifically binds CD30, (ii) exerts a cytostatic or cytotoxic effect on a Hodgkin's Disease cell line, and (iii) comprises a human constant domain, or is not monoclonal antibody AC10 or HeFi-1 and does not result from cleavage of AC10 or HeFi-1 with papain or pepsin. Most preferably, the antibody can exert a cytostatic or cytotoxic effect on the Hodgkin's Disease cell line in the absence of conjugation to a cytostatic or cytotoxic agent, respectively. Moreover, the antibodies of the invention are capable of exerting a cytostatic or cytotoxic effect in the absence of effector cells (such as natural killer cells) and in a complement-independent fashion.

The present invention further provides a protein which (i) competes for binding to CD30 with monoclonal antibody AC10 or HeFi-1, (ii) exerts a cytostatic or cytotoxic effect on a Hodgkin's Disease cell line, and (iii) comprises a human constant domain, or is not monoclonal antibody AC10 or HeFi-1 and does not result from cleavage of AC10 or HeFi-1 with papain or pepsin.

Most preferably, the proteins and antibodies of the invention can exert a cytostatic or cytotoxic effect on the Hodgkin's Disease cell line in the absence of conjugation to a cytostatic or cytotoxic agent, respectively. Additionally, the proteins of the invention are capable of exerting a cytostatic or cytotoxic effect in the absence of effector cells (such as natural killer cells) and in a complement-independent fashion.

The present invention further provides a protein comprising SEQ ID NO:4, SEQ ID NO:6, SEQ ID NO:8, SEQ ID NO:12, SEQ ID NO:14 or SEQ ID NO:16, which protein (i) immunospecifically binds CD30, and (ii) comprises a human constant domain, or is not monoclonal antibody AC10 and does not result from cleavage of AC10 with papain or pepsin.

The present invention yet further provides a protein comprising SEQ ID NO:20, SEQ ID NO:22, SEQ ID NO:24, SEQ ID NO:28, SEQ ID NO:30 or SEQ ID NO:32, which protein (i) immunospecifically binds CD30, and (ii) comprises a human constant domain, or is not monoclonal antibody HeFi-1 and does not result from cleavage of HeFi-1 with papain or pepsin.

The present invention yet further provides a protein comprising an amino acid sequence that has at least 95% identity to SEQ ID NO:2 or SEQ ID NO:10, which protein (i) immunospecifically binds CD30; and (ii) comprises a human constant domain, or is not monoclonal antibody AC10 and does not result from cleavage of AC10 with papain or pepsin.

The present invention yet further provides a protein comprising an amino acid sequence that has at least 95% identity to SEQ ID NO:18 or SEQ ID NO:26, which protein (i) immunospecifically binds CD30; and (ii) comprises a human constant domain, or is not monoclonal antibody HeFi-1 and does not result from cleavage of HeFi-1 with papain or pepsin, in an amount effective for the treatment or prevention of Hodgkin's Disease.

The present invention yet further provides a pharmaceutical composition comprising a therapeutically effective amount of any of the anti-CD30 antibodies of the invention and a pharmaceutically acceptable carrier.

The present invention further provides a pharmaceutical composition comprising (a) an antibody that (i) immunospecifically binds CD30, (ii) exerts a cytostatic or cytotoxic effect on a Hodgkin's Disease cell line, and (iii) comprises a human constant domain, or is not monoclonal antibody AC10 or HeFi-1 and does not result from cleavage of AC10 or HeFi-1 with papain or pepsin, in an amount effective for the treatment or prevention of Hodgkin's Disease; and (b) a pharmaceutically acceptable carrier.

The present invention further provides a pharmaceutical composition comprising (a) a protein, which protein (i) competes for binding to CD30 with monoclonal antibody AC10 or HeFi-1, (ii) exerts a cytostatic or cytotoxic effect on a Hodgkin's Disease cell line, and (iii) comprises a human constant domain, or is not monoclonal antibody AC10 or HeFi-1 and does not result from cleavage of AC10 or HeFi-1 with papain or pepsin, in an amount effective for the treatment or prevention of Hodgkin's Disease; and (b) a pharmaceutically acceptable carrier.

The present invention yet further provides a pharmaceutical composition comprising (a) a protein comprising SEQ ID NO:4, SEQ ID NO:6, SEQ ID NO:8, SEQ ID NO:12, SEQ ID NO:14 or SEQ ID NO:16, which protein (i) immunospecifically binds CD30, and (ii) comprises a human constant domain, or is not monoclonal antibody AC10 and does not result from cleavage of AC10 with papain or pepsin, in an amount effective for the treatment or prevention of Hodgkin's Disease; and (b) a pharmaceutically acceptable carrier.

The present invention yet further provides a pharmaceutical composition comprising (a) a protein comprising SEQ ID NO:20, SEQ ID NO:22, SEQ ID NO:24, SEQ ID NO:28, SEQ ID NO:30 or SEQ ID NO:32, which protein (i) immunospecifically binds CD30, and (ii) comprises a human constant domain, or is not monoclonal antibody HeFi-1 and does not result from cleavage of HeFi-1 with papain or pepsin, in an amount effective for the treatment or prevention of Hodgkin's Disease; and (b) a pharmaceutically acceptable carrier.

The present invention yet further provides a pharmaceutical composition comprising (a) a protein comprising an amino acid sequence that has at least 95% identity to SEQ ID NO:2 or SEQ ID NO:10, which protein (i) immunospecifically binds CD30; and (ii) comprises a human constant domain, or is not monoclonal antibody AC10 and does not result from cleavage of AC10 with papain or pepsin, in an amount effective for the treatment or prevention of Hodgkin's Disease; and (b) a pharmaceutically acceptable carrier.

The present invention yet further provides a pharmaceutical composition comprising: (a) a protein comprising an amino acid sequence that has at least 95% identity to SEQ ID NO:18 or SEQ ID NO:26, which protein (i) immunospecifically binds CD30; and (ii) comprises a human constant domain, or is not monoclonal antibody HeFi-1 and does not result from cleavage of HeFi-1 with papain or pepsin, in an amount effective for the treatment or prevention of Hodgkin's Disease; and (b) a pharmaceutically acceptable carrier.

In certain embodiments, the anti-CD30 antibody of the invention is a monoclonal antibody, a humanized chimeric antibody, a chimeric antibody, a humanized antibody, a glycosylated antibody, a multispecific antibody, a human antibody, a single-chain antibody, a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain. In certain embodiments, the antibody is a bispecific antibody. In other embodiments, the antibody is not a bispecific antibody.

In another preferred embodiment, the protein or antibody is conjugated to a cytotoxic agent. In yet another preferred embodiment, the protein or antibody is a fusion protein comprising the amino acid sequence of a second protein that is not an antibody.

In a specific embodiment, the antibody comprises a human constant domain (e.g., is a human, humanized or chimeric antibody) and is also conjugated to a cytotoxic or a cytostatic agent.

In determining the cytostatic effect of the proteins, including antibodies, of the invention on Hodgkin's Disease cell lines, a culture of the Hodgkin's Disease cell line is contacted with the protein, said culture being of about 5,000 cells in a culture area of about 0.33 cm², said contacting being for a period of 72 hours; exposed to 0.5 μCi of ³H-thymidine during the final 8 hours of said 72-hour period; and the incorporation of ³H-thymidine into cells of the culture, is measured. The protein has a cytostatic or cytotoxic effect on the Hodgkin's Disease cell line if the cells of the culture have reduced ³H-thymidine incorporation compared to cells of the same Hodgkin's Disease cell line cultured under the same conditions but not contacted with the protein.

In one embodiment, the assay for the cytostatic or cytotoxic effect of an antibody of the invention is exhibited upon performing a method comprising (i) immobilizing the antibody in a well, said well having a culture area of about 0.33 cm²; (ii) adding 5,000 cells of the Hodgkin's Disease cell line in the presence of only RPMI with 10% fetal bovine serum or 20% fetal bovine serum to the well; (iii) culturing the cells in presence of only said antibody and RPMI with 10% fetal bovine serum or 20% fetal bovine serum for a period of 72 hours to form a Hodgkin's Disease cell culture; (iv) exposing the Hodgkin's Disease cell culture to 0.5 μCi/well of ³H-thymidine during the final 8 hours of said 72-hour period; and (v) measuring the incorporation of ³H-thymidine into cells of the Hodgkin's Disease cell culture, wherein the antibody has a cytostatic or cytotoxic effect on the Hodgkin's Disease cell line if the cells of the Hodgkin's Disease cell culture have reduced ³H-thymidine incorporation compared to cells of the same Hodgkin's Disease cell line cultured under the same conditions but not contacted with the antibody.

In certain embodiments of the assay, instead of 10% or 20% serum, 0%, 5%, 7.5%, or 15% serum is added to the well. As is standard practice among those skilled in the art, the serum is heat-inactivated prior to its addition to the culture.

Suitable Hodgkin's Disease cell lines to determine the cytostatic or cytotoxic effects of the proteins of the invention are L428, L450, HDLM2 or KM-H2.

One of skill in the art would recognize that there will be slight variation of cell growth and/or thymidine incorporation between Hodgkin's Disease cell cultures that does not relate to the presence of anti-CD30 antibodies. As used herein, the term “reduced ³H-thymidine incorporation” refers to a statistically significant reduction in ³H-thymidine incorporation or a reduction in ³H-thymidine incorporation of at least about 10%. In preferred embodiments, the reduction in ³H-thymidine incorporation is at least a 15%, 20% or 25% reduction. In specific modes of the embodiment, the term “reduced ³H-thymidine incorporation” refers to a reduction of ³H-thymidine incorporation of at least 30%, 35%, 40%, 45%, 50%, 55%, 60%, 65%, 70%, 75%, 80%, 85%, 95% or 95%.

The anti-CD30 antibodies of the invention may or may not have an effect on the shedding of soluble CD30 (“sCD30”) from the surface of a CD30-expressing cell. In certain embodiments, the anti-CD30 antibodies of the invention do not inhibit the shedding of sCD30 by greater than 25%, more preferably no greater than 15% and most preferably no greater than 5%. In other embodiments, the anti-CD30 antibodies of the invention increase the shedding of sCD30, for example by at least 5%, 10%, 15% or 20%. In specific embodiments, the anti-CD30 antibodies of the invention alter the shedding of sCD30 only by −10% to +10% or by −5% to +5%.

Wherein the protein of the invention is an antibody, the antibody is a monoclonal antibody, preferably a recombinant antibody, and most preferably is human, humanized, or chimeric.

The present invention yet further provides an isolated and/or purified nucleic acid comprising a nucleotide sequence encoding a heavy chain of any of the anti-CD30 antibodies of the invention. In certain embodiments, the nucleic acid further encodes the light chain of an anti-CD30 antibody of the invention.

The present invention further provides recombinant cells containing a nucleic acid comprising a nucleotide sequence encoding a heavy chain of any of the anti-CD30 antibodies of the invention. The cell may further contain, in the same or in a separate nucleic acid as that encoding the heavy chain, a nucleic acid encoding the light chain of any of the anti-CD30 antibodies of the invention. The heavy chain and/or the light chain coding sequences are preferably operably linked to a promoter.

Methods of producing the anti-CD30 antibodies (or a heavy or light chain thereof) of the invention, comprising growing the recombinant cells of the invention under conditions such that the antibody (or heavy or light chain) is expressed, and recovering the expressed protein, are alos provided.

The invention further provides isolated nucleic acids encoding a protein, including but not limited to an antibody, that competes for binding to CD30 with monoclonal antibody AC10 or HeFi-1, and exerts a cytostatic or cytotoxic effect on a Hodgkin's Disease cell line. The invention further provides methods of isolating nucleic acids encoding antibodies that immunospecifically bind CD30 and exert a cytostatic or cytotoxic effect on a Hodgkin's Disease cell line. Proteins and antibodies encoded by any of the foregoing nucleic acids are also provided.

The invention further provides a method of producing a protein comprising growing a cell containing a recombinant nucleotide sequence encoding a protein, which protein competes for binding to CD30 with monoclonal antibody AC10 or HeFi-1 and exerts a cytostatic or cytotoxic effect on a Hodgkin's Disease cell line, such that the protein is expressed by the cell; and recovering the expressed protein.

The invention yet further provides a method for identifying an anti-CD30 antibody useful for the treatment or prevention of Hodgkin's Disease, comprising determining whether the anti-CD30 antibody exerts a cytostatic or cytotoxic effect on a Hodgkin's Disease cell line by contacting a culture of the Hodgkin's Disease cell line with the protein, said culture being of about 5,000 cells in a culture area of about 0.33 cm², said contacting being for a period of 72 hours; exposing the culture to 0.5 μCi of ³H-thymidine during the final 8 hours of said 72-hour period; and measuring the incorporation of ³H-thymidine into cells of the culture. The anti-CD30 antibody has a cytostatic or cytotoxic effect on the Hodgkin's Disease cell line and is useful for the treatment or prevention of Hodgkin's Disease if the cells of the culture have reduced ³H-thymidine incorporation compared to cells of the same Hodgkin's Disease cell line cultured under the same conditions but not contacted with the anti-CD30 antibody.

In a specific mode of the embodiment, the method comprises (i) immobilizing the antibody in a well, said well having a culture area of about 0.33 cm²; (ii) adding 5,000 cells of the Hodgkin's Disease cell line in the presence of only RPMI with 10% fetal bovine serum or 20% fetal bovine serum to the well; (iii) culturing the cells in presence of only said antibody and RPMI with 10% fetal bovine serum or 20% fetal bovine serum for a period of 72 hours to form a Hodgkin's Disease cell culture; (iv) exposing the Hodgkin's Disease cell culture to 0.5 μCi/well of ³H-thymidine during the final 8 hours of said 72-hour period; and (v) measuring the incorporation of ³H-thymidine into cells of the Hodgkin's Disease cell culture, wherein the antibody has a cytostatic or cytotoxic effect on the Hodgkin's Disease cell line if the cells of the Hodgkin's Disease cell culture have reduced ³H-thymidine incorporation compared to cells of the same Hodgkin's Disease cell line cultured under the same conditions but not contacted with the antibody.

In certain embodiments of the method, instead of 10% or 20% serum, 0%, 5%, 7.5%, or 15% serum is added to the well.

3.1 Abbreviations

The abbreviation “AEFP” refers to dimethylvaline-valine-dolaisoleuine-dolaproine-phenylalanine-p-phenylenediamine, the auristatin

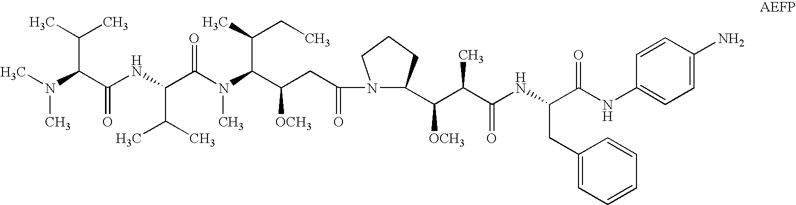

The abbreviation “MMAE” refers to monomethyl auristatin E, the auristatin E derivative depicted below:

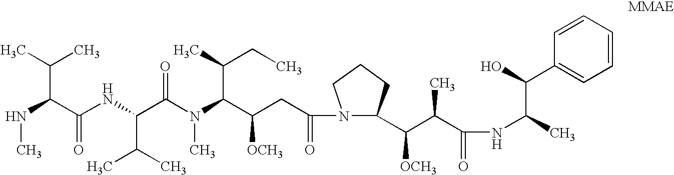

The abbreviation “AEB” refers to an ester produced by reacting auristatin E with paraacetyl benzoic acid, the structure of which is depicted below:

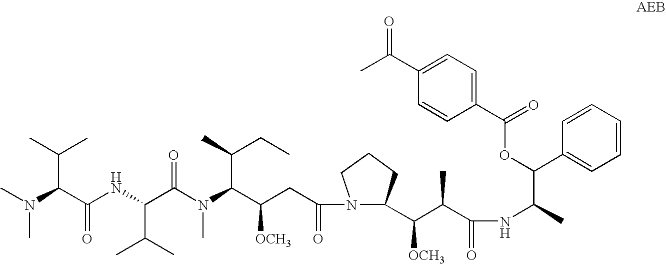

The abbreviation “AEVB” refers to an ester produced by reacting auristatin E with benzoylvaleric acid, the structure of which is depicted below:

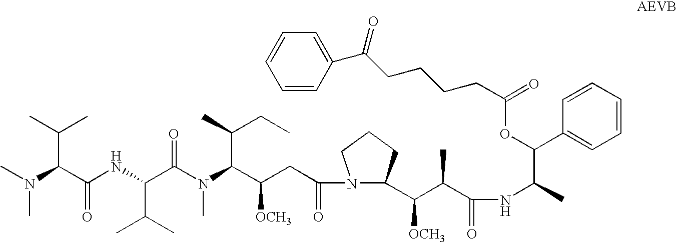

The abbreviations “fk” and “phe-lys” refer to the linker phenylalanine-lysine.

The abbreviations “vc” and “val-cit” refer to the linker valine-citrulline.

### 4. BRIEF DESCRIPTION OF THE FIGURES

FIG. 1. Growth inhibition of Hodgkin's disease cell lines: Hodgkin's disease cell lines HDLM-2, L540, M428 and KM-H2 were cultured at 5×10⁴ cells/well in the presence or absence of 10 μg/ml of immobilized AC10. Ki-1 was used as a control in these assays. Proliferation was measured by ³H-thymidine incorporation following 72 hours of culture.

FIG. 2. Growth inhibition of Hodgkin's disease cell lines: Hodgkin's disease cell lines HDLM-2, L540, L428 and KM-H2 were cultured at 5×10³ cells/well in the presence or absence of 10 μg/ml of immobilized AC10. Ki-1 was used as a control in these assays. Proliferation was measured by ³H-thymidine incorporation following 72 hours of culture.

FIG. 3. Growth inhibition of Hodgkin's disease cell lines: Hodgkin's disease cell lines HDLM-2, L540, L428 and KM-H2 were cultured at 5×10⁴ cells/well in the presence or absence of 0.1 μg/ml AC10 or HeFi-1 that had been cross-linked by the addition of 20 μg/ml polyclonal goat anti-mouse IgG antibodies. Proliferation was measured by ³H-thymidine incorporation following 72 hours of culture.

FIG. 4. Growth inhibition of Hodgkin's disease cell lines: Hodgkin's disease cell lines HDLM-2, L540, L428 and KM-H2 were cultured at 5×10³ cells/well in the presence or absence of 0.1 μg/ml AC10 or HeFi-1 that had been cross-linked by the addition of 20 μg/ml polyclonal goat anti-mouse IgG antibodies. Proliferation was measured by ³H-thymidine incorporation following 72 hours of culture.

FIG. 5. Antitumor activity of AC10 (circles) and HeFi-1 (squares) in disseminated (A) and subcutaneous (B) L540cy Hodgkin's disease xenografts. A) Mice were implanted with 1×10⁷ cells through the tail vein on day 0 and received intraperitoneal injections of antibody at 1 mg/kg/injection using an administration schedule of q2d×10. B) Mice were implanted subcutaneously with 2×10⁷ L540cy cells. When tumors were palpable mice were treated with intraperitoneal injections of AC10 or HeFi-1 at 2 mg/kg/injection q2d×10. In both experiments untreated mice (X) received no therapy.

FIG. 6. Chimeric AC10 expression vector. DNA encoding the heavy chain variable region (Vp) of mAb AC10 was joined to the sequence encoding the human gamma 1 constant region, and the AC10 light chain variable region (VL) was similarly joined to the human kappa constant region in separate cloning vectors. The heavy and light chain chimeric sequences were cloned into plasmid pDEF14 for expression of intact chimeric monoclonal antibody in CHO cells. pDEF14 utilizes the Chinese hamster elongation factor 1 alpha gene promoter which drives transcription of heterologous genes (U.S. Pat. No. 5,888,809).

FIG. 7. Binding saturation of AC10 and chimeric AC10 (cAC10) to CD30-positive Karpas-299. Cells were combined with increasing concentrations of AC10 or cAC10 for 20 minutes, washed with 2% PBS/PBS (staining media) to remove free mAb and incubated with goat-anti-mouse-FITC or goat-anti-human-FITC respectively. The labeled cells were washed again with staining media and examined by flow cytometry. The resultant mean fluorescence intensities were plotted versus mAb concentration as described in Section 9.1.

FIG. 8. In vitro growth inhibition by chimeric AC10 (cAC10). CD30-positive lines and the CD30-negative line HL-60 were plated at 5,000 cells/well. Chimeric AC10 was added at the concentrations noted in the presence of a corresponding 10-fold excess of goat-anti-human IgG. The percent inhibition relative to untreated control wells was plotted versus cAC10 concentration.

FIG. 9. Cell cycle effects of chimeric AC10 on L540cy HD cells. Cells were treated with 1 pg/ml cAC10 and 10 pg/ml of goat anti-human secondary antibody. At the times indicated cells were labeled with BrdU, permeablized and stained with anti-BrdU to detect nascent DNA synthesis (bottom panel), and stained with propidium iodine to detect total DNA content (top panel). Top panels profile G₁, S-phase and G₂ content via P1 staining and the bottom panels show content and DNA synthesis as detected by BrdU incorporation. Regions 2, 5 and 3 designate G₁, S-phase and G₂ respectively. Region 4, containing DNA of sub-G₂ content not undergoing DNA synthesis and region 6, DNA of sub-G₁ content indicate cells with apoptotic DNA fragmentation (Donaldson et al., 1997, J. Immunol. Meth. 203:25-33).

FIG. 10. Efficacy of chimeric AC10 in HD models. (A) Antitumor activity of cAC10 on disseminated L540cy Hodgkin's disease in SCID mice. Groups of mice (five/group) either were left untreated (x) or received 1 (□), 2, (Δ) or 4 (●) mg/kg cAC10 (q4d×5) starting on day 1 after tumor inoculation. (B) Disseminated L540cy Hodgkin's disease in SCID mice where groups mice (five/group) were either were left untreated (x) or received therapy initiated either on day 1 (□), day5 (Δ), or day 9 (●) by cAC10 administered at 4 mg/kg using a schedule of q4d×5. (C) Subcutaneous L540cy HD tumor model in SCID mice. Mice were implanted with 2×10⁷ L540cy Hodgkin's disease cells into the right flank. Groups of mice (five/group) either were left untreated (x) or received 1 (□), 2, (Δ) or 4 (●) mg/kg chimeric AC10 (q4d×5; ▴) starting when the tumor size in each group of 5 animals averaged ˜50 mm³.

FIG. 11. Antitumor activity of chimeric AC10 (cAC10) in subcutaneous L540cy Hodgkin's disease xenografts. SCID mice were implanted subcutaneously with L540cy cells and when the tumors reached an average size of >150 mm³ mice were either left untreated (X) or treated with cAC10 (□) at 2 mg/kg twice per week for 5 injections.

FIG. 12. Delivery of AEB to CD30 positive cells via chimeric AC10. Cells of the indicated cell lines were exposed to chimeric AC10 conjugated to the cytotoxic agent AEB, a derivative of auristatin E (the conjugate is described in U.S. application Ser. No. 09/845,786 filed Apr. 30, 2001, which is incorporated by reference here in its entirety). Cell viability in percent of control is plotted over the concentration of cAC10-drug conjugate that was administered.

FIG. 13. Activity of chimeric AC10 -AEB conjugate on mice bearing L540cy Hodgkin's disease xenografts. Mice were implanted with L540cy cells subcutaneously. Chimeric AC10 conjugated to the cytotoxic agent AEB, a derivative of auristatin E, was administered at indicated doses with a total of 4 doses at 40 day intervals. Tumor volume in mm³ is plotted over days after tumor implantation.

### DETAILED DESCRIPTION OF THE INVENTION

The present invention relates to proteins that bind to CD30 and exert a cytostatic or cytotoxic effect on HD cells. The invention further relates to proteins that compete with AC10 or HeFi-1 for binding to CD30 and exert a cytostatic or cytotoxic effect on HD cells. In one embodiment, the protein is an antibody. In a preferred mode of the embodiment, the antibody is AC10 or HeFi-1, most preferably a humanized or chimeric AC10 or HeFi-1.

The invention further relates to proteins encoded by and nucleotide sequences of AC10 and HeFi-1 genes. The invention further relates to fragments and other derivatives and analogs of such AC10 and HeFi-1 proteins. Nucleic acids encoding such fragments or derivatives are also within the scope of the invention. Production of the foregoing proteins, e.g., by recombinant methods, is provided.

The invention also relates to AC10 and HeFi-1 proteins and derivatives including fusion/chimeric proteins which are functionally active, i.e., which are capable of displaying binding to CD30 and exerting a cytostatic or cytotoxic effect on HD cells.

Antibodies to CD30 encompassed by the invention include human, chimeric or humanized antibodies, and such antibodies conjugated to cytotoxic agents such chemotherapeutic drugs.

The invention further relates to methods of treating or preventing HD comprising administering a composition comprising a protein or nucleic acid of the invention alone or in combination with a cytotoxic agent, including but not limited to a chemotherapeutic drug.

For clarity of disclosure, and not by way of limitation, the detailed description of the invention is divided into the subsections which follow.

5.1 Proteins of the Invention

The present invention encompasses proteins, including but not limited to antibodies, that bind to CD30 and exert cytostatic and/or cytotoxic effects on HD cells. The invention further relates to proteins that compete with AC10 or HeFi-1 for binding to CD30 and exert a cytostatic or cytotoxic effect on HD cells. The cytostatic or cytotoxic effect of the proteins of the invention is preferebly not complement- or effector cell-dependent.

The present invention further encompasses proteins comprising, or alternatively consisting of, a CDR of HeFi-1 (SEQ ID NO:20, SEQ ID NO:22; SEQ ID NO:24; SEQ ID NO:28, SEQ ID NO:30 or SEQ ID NO:32) or AC10 (SEQ ID NO:4; SEQ ID NO:6; SEQ ID NO:8; SEQ ID NO:12; SEQ ID NO:14; or SEQ ID NO:16).

The present invention further encompasses proteins comprising, or alternatively consisting of, a variable region of HeFi-1 (SEQ ID NO:18 or SEQ ID NO:26) or AC10 (SEQ ID NO:2 or SEQ ID NO:10). A table indicating the region of AC10 or HeFi-1 to which each SEQ ID NO corresponds to is provided below:

TABLE 1

| | NUCLEOTIDE | |
|---|---|---|
| | OR | SEQ |
| | AMINO | ID |
| MOLECULE | ACID | NO |
| AC 10 Heavy Chain Variable Region | Nucleotide | 1 |
| AC 10 Heavy Chain Variable Region | Amino Acid | 2 |
| AC 10 Heavy Chain-CDR1(H1) | Nucleotide | 3 |
| AC 10 Heavy Chain-CDR1(H1) | Amino Acid | 4 |
| AC 10 Heavy Chain-CDR2(H2) | Nucleotide | 5 |
| AC 10 Heavy Chain-CDR2(H2) | Amino Acid | 6 |
| AC 10 Heavy Chain-CDR3(H3) | Nucleotide | 7 |
| AC 10 Heavy Chain-CDR3(H3) | Amino Acid | 8 |
| AC 10 Light Chain Variable Region | Nucleotide | 9 |
| AC 10 Light Chain Variable Region | Amino Acid | 10 |
| AC 10 Light Chain-CDR1(L1) | Nucleotide | 11 |
| AC 10 Light Chain-CDR1(L1) | Amino Acid | 12 |
| AC 10 Light Chain-CDR2(L2) | Nucleotide | 13 |
| AC 10 Light Chain-CDR2(L2) | Amino Acid | 14 |
| AC 10 Light Chain-CDR3(L3) | Nucleotide | 15 |
| AC 10 Light Chain-CDR3(L3) | Amino Acid | 16 |
| HeFi-1 Heavy Chain Variable Region | Nucleotide | 17 |
| HeFi-1 Heavy Chain Variable Region | Amino Acid | 18 |
| HeFi-1 Heavy Chain-CDR1(H1) | Nucleotide | 19 |
| HeFi-1 Heavy Chain-CDR1(H1) | Amino Acid | 20 |
| HeFi-1 Heavy Chain-CDR2(H2) | Nucleotide | 21 |
| HeFi-1 Heavy Chain-CDR2(H2) | Amino Acid | 22 |
| HeFi-1 Heavy Chain-CDR3(H3) | Nucleotide | 23 |
| HeFi-1 Heavy Chain-CDR3(H3) | Amino Acid | 24 |
| HeFi-1 Light Chain Variable Region | Nucleotide | 25 |
| HeFi-1 Light Chain Variable Region | Amino Acid | 26 |
| HeFi-1 Light Chain-CDR1(L1) | Nucleotide | 27 |
| HeFi-1 Light Chain-CDR1(L1) | Amino Acid | 28 |
| HeFi-1 Light Chain-CDR2(L2) | Nucleotide | 29 |
| HeFi-1 Light Chain-CDR2(L2) | Amino Acid | 30 |
| HeFi-1 Light Chain-CDR3(L3) | Nucleotide | 31 |
| HeFi-1 Light Chain-CDR3(L3) | Amino Acid | 32 |

The present invention further comprises functional derivatives or analogs of AC10 and HeFi-1. As used herein, the term “functional” in the context of a peptide or protein of the invention indicates that the peptide or protein is 1) capable of binding to CD30 and 2) exerts a cytostatic and/or cytotoxic effect on HD cells.

Generally, antibodies of the invention immunospecifically bind CD30 and exert cytostatic and cytotoxic effects on malignant cells in HD. The cytostatic or cytotoxic effect of the anti-CD30 antibodies of the invention preferably is not complement-dependent and/or is not effector cell-dependent.

The anti-CD30 antibodies of the invention may or may not have an effect on the shedding of soluble CD30 (“sCD30”) from the surface of a CD30-expressing cell, such as a Hodgkin's Disease cell. In certain embodiments, the anti-CD30 antibodies of the invention do not inhibit the shedding of sCD30 by greater than 25%, more preferably no greater than 15% and most preferably no greater than 5%. In other embodiments, the anti-CD30 antibodies of the invention increase the shedding of sCD30, for example by at least 5%, 10%, 15% or 20%. In specific embodiments, the anti-CD30 antibodies of the invention alter the shedding of sCD30 only by −10% to +10% or by −5% to +5%. To determine the effect of an anti-CD30 antibody on the shedding of sCD30, a CD30-expressing cell line, e.g., L540, are pulse labeled with ³⁵S-methionine for 10 minutes, washed, and resuspended in fresh medium. Aliquots (e.g., of 2×10⁵ cells) of the pulse labeled cells are cultured for a chase period of 16 hours with the anti-CD30 antibody, without the antibody or with a control antibody. sCD30 is isolated as described by Hansen et al. (1989, Biol. Chem. Hoppe Seyler 370:409-16), analyzed by SDS-PAGE (7.5-15% gradient gels under reducing conditions) and visualized by autoradiography. The amount of sCD30 can be quantitated by densitometry or by quantitative phorphorimager analysis.

Antibodies of the invention are preferably monoclonal, and may be multispecific, human, humanized or chimeric antibodies, single chain antibodies, Fab fragments, F(ab′) fragments, fragments produced by a Fab expression library, and CD30 binding fragments of any of the above. The term “antibody,” as used herein, refers to immunoglobulin molecules and immunologically active portions of immunoglobulin molecules, i.e., molecules that contain an antigen binding site that immunospecifically binds CD30. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the invention, the antibodies are human antigen-binding antibody fragments of the present invention and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the variable region(s) alone or in combination with the entirety or a portion of the following: hinge region, CH1, CH2, CH3 and CL domains. Also included in the invention are antigen-binding fragments also comprising any combination of variable region(s) with a hinge region, CH1, CH2, CH3 and CL domains. Preferably, the antibodies are human, murine (e.g., mouse and rat), donkey, sheep, rabbit, goat, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulin, as described infra and, for example in U.S. Pat. No. 5,939,598 by Kucherlapati et al.

The antibodies of the present invention may be monospecific, bispecific, trispecific or of greater multispecificity. Multispecific antibodies may be specific for different epitopes of CD30 or may be specific for both CD30 as well as for a heterologous protein. See, e.g., PCT publications WO 93/17715; WO 92/08802; WO 91/00360; WO 92/05793; Tutt, et al., 1991, J. Immunol. 147:60-69; U.S. Pat. Nos. 4,474,893; 4,714,681; 4,925,648; 5,573,920; 5,601,819; Kostelny et al., 1992, J. Immunol. 148:1547-1553.

Antibodies of the present invention may be described or specified in terms of the particular CDRs they comprise. In certain embodiments antibodies of the invention comprise one or more CDRs of AC10 and/or HeFi-1. The invention encompasses an antibody or derivative thereof comprising a heavy or light chain variable domain, said variable domain comprising (a) a set of three CDRs, in which said set of CDRs are from monoclonal antibody AC10 or HeFi-1, and (b) a set of four framework regions, in which said set of framework regions differs from the set of framework regions in monoclonal antibody AC10 or HeFi-1, respectively, and in which said antibody or derivative thereof immunospecifically binds CD30.

In a specific embodiment, the invention encompasses an antibody or derivative thereof comprising a heavy chain variable domain, said variable domain comprising (a) a set of three CDRs, in which said set of CDRs comprises SEQ ID NO:4, 6, or 8 and (b) a set of four framework regions, in which said set of framework regions differs from the set of framework regions in monoclonal antibody AC10, and in which said antibody or derivative thereof immunospecifically binds CD30.

In a specific embodiment, the invention encompasses an antibody or derivative thereof comprising a heavy chain variable domain, said variable domain comprising (a) a set of three CDRs, in which said set of CDRs comprises SEQ ID NO:20, 22 or 24 and (b) a set of four framework regions, in which said set of framework regions differs from the set of framework regions in monoclonal antibody HeFi-1, and in which said antibody or derivative thereof immunospecifically binds CD30.

In a specific embodiment, the invention encompasses an antibody or derivative thereof comprising a light chain variable domain, said variable domain comprising (a) a set of three CDRs, in which said set of CDRs comprises SEQ ID NO:12, 14 or 16, and (b) a set of four framework regions, in which said set of framework regions differs from the set of framework regions in monoclonal antibody AC10, and in which said antibody or derivative thereof immunospecifically binds CD30.

In a specific embodiment, the invention encompasses an antibody or derivative thereof comprising a light chain variable domain, said variable domain comprising (a) a set of three CDRs, in which said set of CDRs comprises SEQ ID NO:28, 30, or 32, and (b) a set of four framework regions, in which said set of framework regions differs from the set of framework regions in monoclonal antibody HeFi-1, and in which said antibody or derivative thereof immunospecifically binds CD30.

Additionally, antibodies of the present invention may also be described or specified in terms of their primary structures. Antibodies having at least 50%, at least 55%, at least 60%, at least 65%, at least 70%, at least 75%, at least 80%, at least 85%, at least 90%, at least 95% and most preferably at least 98% identity (as calculated using methods known in the art and described herein) to the variable regions and AC10 or HeFi-1 are also included in the present invention. Antibodies of the present invention may also be described or specified in terms of their binding affinity to CD30. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×⁻¹³ M, 10⁻¹³ M, 5×10⁻¹⁴ M, 10⁻¹⁴ M, 5×10⁻¹⁵ M, or 10⁻¹⁵ M.

The antibodies and proteins of the invention can be purified, for example by affinity chromatography with the CD30 antigen. In certain embodiments, the antibody is at least 50%, at least 60%, at least 70% or at least 80% pure. In other embodiments, the antibody is more than 85% pure, more than 90% pure, more than 95% pure or more than 99% pure.

The antibodies of the invention include derivatives that are modified, i.e, by the covalent attachment of any type of molecule to the antibody such that covalent attachment does not prevent the antibody from binding to CD30 or from exerting a cytostatic or cytotoxic effect on HD cells. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to CD30 can be produced by various procedures well known in the art. For example, CD30 can be administered to various host animals including, but not limited to, rabbits, mice, rats, etc. to induce the production of sera containing polyclonal antibodies specific for the protein. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a non-limiting example, mice can be immunized with CD30 or a cell expressing CD30 or a fragment or derivative thereof. Once an immune response is detected, e.g., antibodies specific for CD30 are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding CD30. Ascites fluid, which generally contains high levels of antibodies, can be generated by injecting mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind to CD30.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH 1 domain of the heavy chain.

For example, the antibodies of the present invention can also be generated using various phage display methods known in the art. In phage display methods, functional antibody domains are displayed on the surface of phage particles which carry the nucleic acid sequences encoding them. In a particular embodiment, such phage can be utilized to display antigen binding domains expressed from a repertoire or combinatorial antibody library (e.g., human or murine). In phage display methods, functional antibody domains are displayed on the surface of phage particles which carry the nucleic acid sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 binding domains expressed from phage with Fab, Fv or disulfide stabilized Fv antibody domains recombinantly fused to either the phage gene III or gene VIII protein. Phage expressing an antigen binding domain that binds to CD30 or an AC10 or HeFi-binding portion thereof can be selected or identified with antigen e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology, 191-280; PCT Application No. PCT/GB91/O1 134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 1992, 12(6):864-869; and Sawai et al., 1995, AJRI 34:26-34; and Better et al., 1988, Science 240:1041-1043 (said references incorporated by reference in their entireties).

Examples of techniques which can be used to produce single-chain Fvs and antibodies include those described in U.S. Pat. Nos. 4,946,778 and 5,258,498; Huston et al., 1991, Methods in Enzymology 203:46-88 ; Shu et al., 1993, PNAS 90:7995-7999; and Skerra et al., 1988, Science 240:1038-1040. For some uses, including in vivo use of antibodies in humans and in vitro proliferation or cytotoxicity assays, it is preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202 ; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entirety. Humanized antibodies are antibody molecules from non-human species antibody that binds the desired antigen having one or more CDRs from the non-human species and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323, which are incorporated herein by reference in their entireties.) Antibodies can be humanized using a variety of techniques known in the art including, for example, CDR-grafting (EP 239,400; PCT publication WO 9 1/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al., 1994, PNAS 91:969-973), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which express human immunoglobulin genes. For example, the human heavy and light chain immunoglobulin gene complexes may be introduced randomly or by homologous recombination into mouse embryonic stem cells. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of CD30. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Pat. No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., 1994, Bio/technology 12:899-903).

Further, antibodies to CD30 can, in turn, be utilized to generate anti-idiotype antibodies that “mimic” proteins of the invention using techniques well known to those skilled in the art. (See, e.g., Greenspan & Bona, 1989, FASEB J. 7(5):437-444; and Nissinoff, 1991, J. Immunol. 147(8):2429-2438). Fab fragments of such anti-idiotypes can be used in therapeutic regimens to elicit an individual's own immune response against CD30 and HD cells.

As alluded to above, proteins that are therapeutically or prophylactically useful against HD need not be antibodies. Accordingly, proteins of the invention may comprise one or more CDRs from an antibody that binds to CD30 and exerts a cytotoxic and/or cytostatic effect on HD cells. Preferably, a protein of the invention is a multimer, most preferably a dimer.

The invention also provides proteins, including but not limited to antibodies, that competitively inhibit binding of AC10 or HeFi-1 to CD30 as determined by any method known in the art for determining competitive binding, for example, the immunoassays described herein. In preferred embodiments, the protein competitively inhibits binding of AC10 or HeFi-1 to CD30 by at least 50%, more preferably at least 60%, yet more preferably at least 70%, and most preferably at least 75%. In other embodiments, the protein competitively inhibits binding of AC10 or HeFi-1 to CD30 by at least 80%, at least 85%, at least 90%, or at least 95%.

As discussed in more detail below, the proteins of the present invention may be used either alone or in combination with other compositions in the prevention or treatment of HD. The proteins may further be recombinantly fused to a heterologous protein at the N- or C-terminus or chemically conjugated (including covalently and non-covalently conjugations) to cytotoxic agents, proteins or other compositions. For example, antibodies of the present invention may be recombinantly fused or conjugated to molecules useful as chemotherapeutics or toxins, or comprise a radionuclide for use as a radio-therapeutic. See, e.g., PCT publications WO 92/08495; WO 91/14438; WO 89/12624; U.S. Pat. No. 5,314,995; and EP 396,387.

Proteins of the invention may be produced recombinantly by fusing the coding region of one or more of the CDRs of an antibody of the invention in frame with a sequence coding for a heterologous protein. The heterologous protein may provide one or more of the following characteristics: added therapeutic benefits; promote stable expression of the protein of the invention; provide a means of facilitating high yield recombinant expression of the protein of the invention; or provide a multimerization domain.

In addition to proteins comprising one or more CDRs of an antibody of the invention, proteins of the invention may be identified using any method suitable for screening for protein-protein interactions. Initially, proteins are identified that bind to CD30, then their ability to exert a cytostatic or cytotoxic effect on HD cells can be determined. Among the traditional methods which can be employed are “interaction cloning” techniques which entail probing expression libraries with labeled CD30 in a manner similar to the technique of antibody probing of λgt11 libraries, supra. By way of example and not limitation, this can be achieved as follows: a cDNA clone encoding CD30 (or an AC10 or HeFi-1 binding domain thereof) is modified at the terminus by inserting the phosphorylation site for the heart muscle kinase (HMK) (Blanar & Rutter, 1992, Science 256:1014-1018). The recombinant protein is expressed in E. coli and purified on a GDP-affinity column to homogeneity (Edery et al., 1988, Gene 74:517-525) and labeled using γ³²P-ATP and bovine heart muscle kinase (Sigma) to a specific activity of 1×10⁸ cpm/μg, and used to screen a human placenta λgt11 cDNA library in a “far-Western assay” (Blanar & Rutter, 1992, Science 256:1014-1018). Plaques which interact with the CD30 probe are isolated. The cDNA inserts of positive λ plaques are released and subcloned into a vector suitable for sequencing, such as pBluescript KS (Stratagene).

One method which detects protein interactions in vivo, the two-hybrid system, is described in detail for illustration purposes only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to CD30, and the other consists of the activator protein's activation domain fused to an unknown protein that is encoded by a cDNA which has been recombined into this plasmid as part of a cDNA library. The plasmids are transformed into a strain of the yeast Saccharomyces cerevisiae that contains a reporter gene (e.g., lacZ) whose regulatory region contains the transcription activator's binding sites. Either hybrid protein alone cannot activate transcription of the reporter gene, the DNA-binding domain hybrid cannot because it does not provide activation function, and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology can be used to screen activation domain libraries for proteins that interact with CD30, which in this context is a “bait” gene product. Total genomic or cDNA sequences are fused to the DNA encoding an activation domain. This library and a plasmid encoding a hybrid of a CD30 coding region (for example, a nucleotide sequence which codes for a domain of CD30 known to interact with HeFi-1 or AC10) fused to the DNA-binding domain are co-transformed into a yeast reporter strain, and the resulting transformants are screened for those that express the reporter gene. For example, and not by way of limitation, the CD30 coding region can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

Once a CD30-binding protein is identified, its ability (alone or when multimerized or fused to a dimerization or multimerization domain) to elicit a cytostatic or cytotoxic effect on HD cells is determined by contacting a culture of an HD cell line, such as L428, L450, HDLM2 or KM-H2, with the protein. Culture conditions are most preferably about 5,000 cells in a culture area of about 0.33 cm², and the contacting period being approximately 72 hours. The culture is then exposed to 0.5 μCi of ³H-thymidine during the final 8 hours of the 72-hour period and the incorporation of ³H-thymidine into cells of the culture is measured. The protein has a cytostatic or cytotoxic effect on the HD cell line if the cells of the culture have reduced ³H-thymidine incorporation compared to cells of the same cell line cultured under the same conditions but not contacted with the protein.

Without limitation as to mechanism of action, a protein of the invention preferably has more than one CD30-binding site and therefore a capacity to cross link CD30 molecules. Proteins which bind to CD30 or compete for binding to CD30 with AC10 or HeFi-1 can acquire the ability to induce cytostatic or cytotoxic effects on HD cells if dimerized or multimerized. Wherein the CD30-binding protein is a monomeric protein, it can be expressed in tandem, thereby resulting in a protein with multiple CD30 binding sites. The CD30-binding sites can be separated by a flexible linker region. In another embodiment, the CD30-binding proteins can be chemically cross-linked, for example using gluteraldehyde, prior to administration. In a preferred embodiment, the CD30-binding region is fused with a heterologous protein, wherein the heterologous protein comprises a dimerization and multimerization domain. Prior to administration of the protein of the invention to a subject for the purpose of treating or preventing HD, such a protein is subjected to conditions that allows formation of a homodimer or heterodimer. A heterodimer, as used herein, may comprise identical dimerization domains but different CD30-binding regions, identical CD30-binding regions but different dimerization domains, or different CD30-binding regions and dimerization domains.

Particularly preferred dimerization domains are those that originate from transcription factors.

In one embodiment, the dimerization domain is that of a basic region leucine zipper (“bZIP”). bZIP proteins characteristically possess two domains—a leucine zipper structural domain and a basic domain that is rich in basic amino acids, separated by a “fork” domain (C. Vinson et al., 1989, Science, 246:911-916). Two bZIP proteins dimerize by forming a coiled coil region in which the leucine zipper domains dimerize. Accordingly, these coiled coil regions may be used as fusion partners for the proteins and the invention.

Particularly useful leucine zipper domain are those of the yeast transcription factor GCN4, the mammalian transcription factor CCAAT/enhancer-binding protein C/EBP, and the nuclear transform in oncogene products, Fos and Jun (see Landschultz et al., 1988, Science 240:1759-1764; Baxevanis and Vinson, 1993, Curr. Op. Gen. Devel., 3:278-285; and O'Shea et al., 1989, Science, 243:538-542).

In another embodiment, the dimerization domain is that of a basic-region helix-loop-helix (“bHLH”) protein (Murre et al, 1989, Cell, 56:777-783). bHLH proteins are also composed of discrete domains, the structure of which allows them to recognize and interact with specific sequences of DNA. The helix-loop-helix region promotes dimerization through its amphipathic helices in a fashion analogous to that of the leucine zipper region of the bZIP proteins (Davis et al., 1990 Cell, 60:733-746; Voronova and Baltimore, 1990 Proc. Natl. Acad. Sci. USA, 87:4722-4726). Particularly useful hHLH proteins are myc, max, and mac.

Heterodimers are known to form between Fos and Jun (Bohmann et al., 1987, Science, 238:1386-1392), among members of the ATF/CREB family (Hai et al.,1989, Genes Dev., 3:2083-2090), among members of the C/EBP family (Cao et al., 1991, Genes Dev., 5:1538-1552; Williams et al., 1991, Genes Dev., 5:1553-1567; and Roman et al., 1990, Genes Dev., 4:1404-1415), and between members of the ATF/CREB and Fos/Jun families Hai and Curran, 1991, Proc. Natl. Acad. Sci. USA, 88:3720-3724). Therefore, when a protein of the invention is administered to a subject as a heterodimer comprising different dimerization domains, any combination of the foregoing may be used.

5.2 Binding Assays

As described above, the proteins, including antibodies, of the invention bind to CD30 and exert a cytostatic or cytotoxic effect on HD cells. Methods of demonstrating the ability of a protein of the invention to bind to CD30 are described herein.

The antibodies of the invention may be assayed for immunospecific binding to CD30 by any method known in the art. The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et. al., eds., 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody to the cell lysate, incubating for a period of time (e.g., 1-4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody to immunoprecipitate CD30 can be assessed by, e.g., Western blot analysis. One of skill in the art would be knowledgeable as to the parameters that can be modified to increase the binding of the antibody to CD30 and decrease the background (e.g., pre-clearing the cell lysate with sepharose beads). For further discussion regarding immunoprecipitation protocols see, e.g., Ausubel et al., eds., 1994, Current Protocols in Molecular Biology, Vol. 1, John Wiley & Sons, Inc., New York at 10. 16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), blocking the membrane with primary antibody (i.e., the putative anti-CD30 antibody) diluted in blocking buffer, washing the membrane in washing buffer, incubating the membrane with a secondary antibody (which recognizes the primary antibody, e.g., an anti-human antibody) conjugated to an enzyme substrate (e.g., horseradish peroxidase or alkaline phosphatase) or radioactive molecule (e.g., ³²p or ¹²⁵I) diluted in blocking buffer, washing the membrane in wash buffer, and detecting the presence of the secondary antibody. One of skill in the art would be knowledgeable as to the parameters that can be modified to increase the signal detected and to reduce the background noise. For further discussion regarding Western blot protocols see, e.g., Ausubel et al., eds., 1994, Current Protocols in Molecular Biology, Vol. 1, John Wiley & Sons, Inc., New York at 10.8.1.

ELISAs comprise preparing antigen (i.e., CD30), coating the well of a 96 well microtiter plate with the CD30, adding the antibody conjugated to a detectable compound such as an enzyme (e.g., horseradish peroxidase or alkaline phosphatase) to the well and incubating for a period of time, and detecting the presence of the antibody. In ELISAs the antibody does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of CD30 protein to the coated well. One of skill in the art would be knowledgeable as to the parameters that can be modified to increase the signal detected as well as other variations of ELISAs known in the art. For further discussion regarding ELISAs see, e.g., Ausubel et al., eds., 1994, Current Protocols in Molecular Biology, Vol. 1, John Wiley & Sons, Inc., New York at 11.2.1.

The binding affinity of an antibody to CD30 and the off-rate of an antibody CD30 interaction can be determined by competitive binding assays. One example of a competitive binding assay is a radioimmunoassay comprising the incubation of labeled CD30 (e.g., ³H or ¹²⁵I) with the antibody of interest in the presence of increasing amounts of unlabeled CD30, and the detection of the antibody bound to the labeled CD30. The affinity of the antibody for CD30 and the binding off-rates can then be determined from the data by Scatchard plot analysis. Competition with a second antibody (such as AC10 or HeFi-1) can also be determined using radioimmunoassays. In this case, CD30 is incubated with the antibody of interest conjugated to a labeled compound (e.g., ³H or 125I) in the presence of increasing amounts of an unlabeled second antibody.

Proteins of the invention may also be assayed for their ability to bind to CD30 by a standard assay known in the art. Such assays include far Westerns and the yeast two hybrid system. These assays are described in Section 5.2, supra. Another variation on the far Western technique described above entails measuring the ability of a labeled candidate protein to bind to CD30 in a Western blot. In one non-limiting example of a far Western blot, CD30 or the fragment thereof of interest is expressed as a fusion protein further comprising glutathione-S-transferase (GST) and a protein serine/threonine kinase recognition site (such as a cAMP-dependent kinase recognition site). The fusion protein is purified on glutathione-Sepharose beads (Pharmacia Biotech) and labeled with bovine heart kinase (Sigma) and 100 μCi of ³²P-ATP (Amersham). The test protein(s) of interest are separated by SDS-PAGE and blotted to a nitrocellulose membrane, then incubated with the labeled CD30. Thereafter, the membrane is washed and the radioactivity quantitated. Conversely, the protein of interest can be labeled by the same method and used to probe a nitrocellulose membrane onto which CD30 has been blotted.

5.3 Assays for Cytotoxic and Cytostatic Activities

By definition, a protein of the invention must exert a cytostatic or cytotoxic effect on a cell of HD. Suitable HD cell lines for this purpose include L428, L450, HDLM2 and KM-H2 (all of which are available from the German Collection of Microorganisms and Cell Cultures (DMSZ: Deutsche Sammlung von Mikroorganismen und Zellkulturen GmbH)).

Many methods of determining whether a protein exerts a cytostatic or cytotoxic effect on a cell are known to those of skill in the art, and can be used to elucidate whether a particular protein is a protein of the invention. Illustrative examples of such methods are described below.

Wherein a protein that binds to CD30 does not exert a cytostatic or cytotoxic effect on HD cells, the protein can be multimerized according to the methods described in Section 5.1, supra, and the multimer assayed for its ability to exert a cytostatic or cytotoxic effect on HD cells.

Once a protein is identified that both (i) binds to CD30 and (ii) exerts a cytostatic or cytotoxic effect on HD cells, its therapeutic value is validated in an animal model, as described in Section 6, infra.

In a preferred embodiment, determining whether a protein exerts a cytostatic or cytotoxic effect on a HD cell line can be made by contacting a 5,000 cell-culture of the HD cell line in a culture area of about 0.33 cm² with the protein for a period of 72 hours. During the last 8 hours of the 72-hour period, the culture is exposed to 0.5 μCi of ³H-thymidine. The incorporation of ³H-thymidine into cells of the culture is then measured. The protein has a cytostatic or cytotoxic effect on the HD cell line and is useful for the treatment or prevention of HD if the cells of the culture contacted with the protein have reduced ³H-thymidine incorporation compared to cells of the same HD cell line cultured under the same conditions but not contacted with the anti-CD30 antibody.

In a specific mode of the embodiment, the method comprises (i) immobilizing the antibody in a well, said well having a culture area of about 0.33 cm²; (ii) adding 5,000 cells of the Hodgkin's Disease cell line in the presence of only RPMI with 10% fetal bovine serum or 20% fetal bovine serum to the well; (iii) culturing the cells in presence of only said antibody and RPMI with 10% fetal bovine serum or 20% fetal bovine serum for a period of 72 hours to form a Hodgkin's Disease cell culture; (iv) exposing the Hodgkin's Disease cell culture to 0.5 μCi/well of ³H-thymidine during the final 8 hours of said 72-hour period; and (v) measuring the incorporation of ³H-thymidine into cells of the Hodgkin's Disease cell culture, wherein the antibody has a cytostatic or cytotoxic effect on the Hodgkin's Disease cell line if the cells of the Hodgkin's Disease cell culture have reduced ³H-thymidine incorporation compared to cells of the same Hodgkin's Disease cell line cultured under the same conditions but not contacted with the antibody.

In certain embodiments of the method for determining the cytotoxic or cytostatic effect of the anti-CD30 antibodies of the invention, instead of 10% or 20% serum, 0%, 5%, 7.5%, or 15% serum is added to the well. As is standard practice among those skilled in the art, the serum is heat-inactived prior to its addition to the culture.

There are many other cytotoxicity assays known to those of skill in the art. Some of these assays measure necrosis, while others measure apoptosis (programmed cell death). Necrosis is accompanied by increased permeability of the plasma membrane; the cells swell and the plasma membrane ruptures within minutes. On the other hand, apoptosis is characterized by membrane blebbing, condensation of cytoplasm and the activation of endogenous endonucleases. Only one of these effects on HD cells is sufficient to show that a CD30-binding protein is useful in the treatment or prevention of HD as an alternative to the assays measuring cytostatic or cytotoxic effects described above.

In one embodiment, necrosis measured by the ability or inability of a cell to take up a dye such as neutral red, trypan blue, or ALAMAR™ blue (Page et al., 1993, Intl. J. of Oncology 3:473-476). In such an assay, the cells are incubated in media containing the dye, the cells are washed, and the remaining dye, reflecting cellular uptake of the dye, is measured spectrophotometrically.

In another embodiment, the dye is sulforhodamine B (SRB), whose binding to proteins can be used as a measure of cytotoxicity (Skehan et al., 1990, J. Nat'l Cancer Inst. 82:1107-12).

In yet another embodiment, a tetrazolium salt, such as MTT, is used in a quantitative colorimetric assay for mammalian cell survival and proliferation by detecting living, but not dead, cells (see, e.g., Mosmann, 1983, J. Immunol. Methods 65:55-63).

In yet another embodiment, apoptotic cells are measured in both the attached and “floating” compartments of the cultures. Both compartments are collected by removing the supernatant, trypsinizing the attached cells, and combining both preparations following a centrifugation wash step (10 minutes, 2000 rpm). The protocol for treating tumor cell cultures with sulindac and related compounds to obtain a significant amount of apoptosis has been described in the literature (see, e.g., Piazza et al., 1995, Cancer Research 55:3110-16). Features of this method include collecting both floating and attached cells, identification of the optimal treatment times and dose range for observing apoptosis, and identification of optimal cell culture conditions.

In yet another embodiment, apoptosis is quantitated by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

In yet another embodiment, apoptosis can be observed morphologically. Following treatment with a test protein or nucleic acid, cultures can be assayed for apoptosis and necrosis by fluorescent microscopy following labeling with acridine orange and ethidium bromide. The method for measuring apoptotic cell number has previously been described by Duke & Cohen, 1992, Current Protocols In Immunology, Coligan et al., eds., 3.17.1-3.17.16. In another mode of the embodiment, cells can be labeled with the DNA dye propidium iodide, and the cells observed for morphological changes such as chromatin condensation and margination along the inner nuclear membrane, cytoplasmic condensation, increased membrane blebbing and cellular shrinkage.

In yet another embodiment, cytotoxic and/or cytostatic effects can be determined by measuring the rate of bromodeoxyuridine incorporation. The cells are cultured in complete media with a test protein or nucleic acid. At different times, cells are labeled with bromodeoxyuridine to detect nascent DNA synthesis, and with propidium iodine to detect total DNA content. Labeled cells are analyzed for cell cycle position by flow cytometry using the Becton-Dickinson Cellfit program as previously described (Donaldson et al., 1997, J. Immunol. Meth. 203:25-33). An example of using bromodeoxyuridine incorporation to determine the cytostatic and/or cytotoxic effects of the anti-CD30 antibodies of the invention is described in Section 9, infra.

5.4 Assays for Signaling Activity

In certain preferred embodiments, a protein of the invention is capable of inducing one or more hallmarks of signaling through CD30 upon binding to a CD30-expressing lymphocyte. CD30-expressing lymphocytes that can be assayed for a signaling effect of a CD30 binding protein may be cultured cell lines (e.g., Jurkat and CESS, both of which are available from the ATCC; or Karpas 299 and L540, both of which are available from Deutsche Sammlung von Mikroorganismen und Zellkulturen GmbH), or lymphocytes prepared from a fresh blood sample.

In a preferred embodiment, the proteins of the invention are cross-linked prior to assessing their activity on activated lymphocytes. In an exemplary embodiment, where the protein of the invention is an anti-CD30 antibody, the anti-CD30 antibody can be cross-linked in solution. Briefly, one or more dilutions of the anti-CD30 antibody can be titrated into 96-well flat bottom tissue culture plates in the absence or presence of secondary antibodies. Lymphocytes are then added to the plates at approximately 5,000 cells/well. The signaling activity of the antibody can then be assessed as described herein.

Many methods of determining whether a protein induces one or more hallmarks of signaling through CD30 are known to those of skill in the art. Illustrative examples of such methods are described below.

5.4.1 Calcium Release

In one embodiment, a protein of the invention can induce the release of intracellular free Ca2+ in Jurkat cells when it is cross-linked, for example with a secondary antibody. The release of intracellular free Ca2+ can be measured as described by Ellis et al. (1993, J. Immunol., 151, 2380-2389) or by Mond and Brunswick (1998, Current Protocols in Immunology, Unit 3.9, Wiley).

5.4.2 Traf Localization

Four TNF receptor-associated factors (TRAFs) including TRAF1, TRAF2, TRAF3, and TRAF5 have been demonstrated to interact with the cytoplasmic tail of CD30 (Gedrich et al., 1996, J. Biol. Chem., 271, 12852-12858; Lee et al., 1996, Proc. Natl. Acad. Sci. USA., 93, 9699-9703; Ansieau et al., 1996, Proc. Natl. Acad. Sci. USA., 93, 14053-14058; Aizawa et al., 1997, J. Biol. Chem., 272, 2042-2045; Tsitsikov et al., 1997, Proc. Natl. Acad. Sci. USA., 94, 1390-1395; Lee et al., 1997, J. Exp. Med., 185, 1275-1285; Duckett and Thompson, 1997, Genes Dev., 11, 2810-2821). Using co-transfection studies, yeast two-hybrid screening, and GST fusion proteins, the TRAF interacting sites have been mapped to the carboxyl terminal of the cytoplasmic tail of CD30, and the association between CD30 and the TRAFs in the cytosolic phase has been hypothesized to be a key event in the CD30-mediated signal cascade. The interaction between CD30 and TRAF does not appear to require CD30 ligation (Ansieau et al., 1996, Proc. Natl. Acad. Sci. USA., 93, 14053-14058; Aizawa et al., 1997, J. Biol. Chem., 272, 2042-2045). However, cross-linking of CD30 leads to a disappearance of TRAF1 and TRAF2 from the detergent-soluble fractions of cell lysates (Duckett and Thompson, 1997, Genes Dev., 11, 2810-2821; Arch et al., 2000, Biochem. Biophys. Res. Commun., 272, 936-945). The disappearance of TRAF2 is accompanied by a corresponding increase in the quantity of TRAF2 detectable in the detergent-insoluble fraction containing the nuclei (Arch et al., 2000, Biochem. Biophys. Res. Commun., 272, 936-945). Further subcellular localization studies have confirmed that cross-linking of CD30 induces a translocation of TRAF2 from the cytosol to the perinuclear region of cells (Arch et al., 2000, Biochem. Biophys. Res. Commun., 272, 936-945). Such CD30-mediated translocation of TRAF2 is hypothesized to modulate cell survival by regulating the sensitivity of cells to undergo apoptosis induced by other TRAF-binding members of the TNF receptor superfamily (Duckett and Thompson, 1997, Genes Dev., 11, 2810-2821; Arch et al., 2000, Biochem. Biophys. Res. Commun., 272, 936-945).

To determine whether an antibody of the invention induces nuclear translocation of TRAF2, the antibody of the invention is contacted with CD30+ cells and a cross-linking agent, such as a secondary antibody. Confocal microscopy can then be used to compare localization of TRAF2 in cells incubated with the antibody of the invention (plus cross-linking reagent) versus cells not incubated with the antibody of the invention.

In an alternative embodiment, whether an antibody of the invention induces TRAF2 nuclear localization can be assayed by measuring the amount of TRAF2 in various cell fractions, for example on a Western Blot. For example, 2 μg/ml of an antibody of the invention can be incubated with CD30⁺ cells at 0.5×10⁶/ml. The antibody is cross-linked by 20 μg/ml of a secondary antibody (e.g., where the antibody of the invention is a mouse monoclonal antibody, a goat anti-mouse IgG Fe specific antibody (Jackson ImmunoReseach, West Grove, Pa.) can be used as a secondary antibody) at 37° C. and 5% CO₂. At designated time-points (e.g., 2 to 24 hours), 5×10⁶ cells are removed and spun down. After two washes with ice-cold PBS, cells are lysed at 100×10⁶/ml in a lysis buffer (0.15 M NaCl, 0.05 M Tris-HCl, pH 8.0, 0.005 M EDTA, and 0.5% NP-40 or Triton X-100) supplemented with a protease inhibitor cocktail (Roche Diagnositc GmBH, Mannheim, Germany). Lysis is done at 4° C. for 2 hours with constant mixing. After lysis, the detergent-soluble and detergent-insoluble fractions are separated by centrifugation at 14,000×g for 20 minutes. The detergent-soluble fraction is then transferred to a separate tube and an equal volume of 2×SDS-PAGE reducing sample buffer is added to it. An equal volume of 1×SDS-PAGE reducing sample buffer is also added to the detergent-insoluble fraction, i.e., the pellet after centrifugation. Both fractions are heated to 100° C. for 2 minutes. About 10 μl of the fractions from each time point is then resolved by 12% Tris-glycine SDS-PAGE (Invitrogen, Carlsbad, Calif.). Resolved proteins are Western-transferred onto PVDF membranes (Invitrogen), which is blocked with Tris buffer saline (0.05 M Tris-HCl, pH 8.0, 0.138 M NaCl, 0.0027 M KCl) supplemented with 0.05% Tween 20 and 5% BSA. The blots are immunoblotted with an anti-TRAF2 antibody (Santa Cruz, San Diego, Calif.). The presence of TRAF2 protein in the different fractions is detected by horseradish peroxidase (HRP)-conjugated F(ab′)₂ goat anti-rabbit IgG Fc (Jackson ImmunoResearch) and the peroxidase substrate kit DAB (Vector Laboratories, Burlingame, Calif.). Alternatively, the SuperSignal West Pico Chemiluminescent Substrate kit (Pierce, Rockford, Ill.) can also be used for detection.

5.4.3 NF-κB Activation

Another well-defined signal transduction event that can be induced by certain antibodies of the invention is the activation of NF-κB. Anti-CD30 mAbs including M44, M67, and Ber-H2 can activate NF-κB as detected by standard mobility shift DNA-binding assay (McDonald et al., 1995, Eur. J. Immunol., 25, 2870-2876; Ansieau et al., 1996, Proc. Natl. Acad. Sci. USA., 93, 14053-14058; Horie et al., 1998, Int. Immunol., 10, 203-210). Such effect can be observed in Hodgkin cells, T cells, and transfectant expressing CD30 (McDonald et al., 1995, Eur. J. Immunol., 25, 2870-2876; Biswas et al., 1995, Immunity, 2, 587-596; Ansieau et al., 1996, Proc. Natl. Acad. Sci. USA., 93, 14053-14058; Horie et al., 1998, Int. Immunol., 10, 203-210). Initial mapping studies revealed that the interaction between TRAF1, TRAF2, and TRAF5 with the cytoplasmic tail of CD30 was required for the CD30-mediated activation of NF-κB (Lee et al., 1996, Proc. Natl. Acad. Sci. USA., 93, 9699-9703; Ansieau et al., 1996, Proc. Natl. Acad. Sci. USA., 93, 14053-14058; Aizawa et al., 1997, J. Biol. Chem., 272, 2042-2045; Duckett et al., 1997, Mol. Cell. Biol., 17, 1535-1542). More recently, evidence has become available that ligation of CD30 by agonistic mAbs can also activate NF-κB via a TRAF2/5-independent pathway (Horie et al., 1998, Int. Immunol., 10, 203-210). Some of the biological consequences of the CD30-mediated activation of NF-κB include activation of gene transcription (Biswas et al., 1995, Immunity, 2, 587-596; Maggi et al., 1995, Immunity, 3, 251-255) and regulation of cell survival (Mir et al., 2000, Blood, 96, 4307-4312; Horie et al., 2002, Oncogene, 21, 2439-2503). Any of these characteristics of NF-κB activation can be assayed to determine whether an antibody of the invention induces one or more hallmarks of CD30 signaling.

Whether NF-κB activation is induced in CD30⁺ cells by an antibody of the invention can be measured by, for example, incubating CD30⁺ cells at 3×10⁶/ml with the antibody at 2 μg/ml, the antibody then cross-linked (e.g., where the antibody is a mouse monoclonal antibody, the antibody can be cross-linked by 20 μg/ml of a goat anti-mouse IgG Fc specific antibody (Jackson ImmunoReseach, West Grove, Pa.)) and the culture incubated at 37° C. and 5% CO₂ for 1 hour with constant shaking. The cell density is adjusted to 1.2×10⁶/ml, and incubation with shaking is carried on for an additional hour. Thereafter, cell density is further reduced to 0.6×10⁶/ml, and cells are incubated for an additional 46 hours at 37° C. and 5% CO₂ without any further shaking. At the end of incubation, nuclear extracts can be prepared from stimulated cells and analyzed for NF-κB activation.

NF-κB activation is assayed by collecting the cells by centrifugation at 1850×g for 20 minutes and then washing them once in 5 packed cell volumes of PBS. The cell pellet is resuspended in 5 packed cell volumes of a hypotonic buffer (0.01 M Hepes, pH 7.9, 0.0015 M MgCl₂, 0.01 M KCl, 0.0002 M phenylmethyl sulphonyl fluoride, 0.0005 M dithiothreitol). Cells are collected by centrifugation at 1850×g for 5 minutes. The pellet is then resuspended in 3 packed cell volumes of the hypotonic buffer and allowed to swell on ice for 10 minutes. After that, swollen cells are homogenized with slow up-and-down strokes in a Dounce homogenizer, using a tight B pestle. Cell lysis is monitored by trypan blue exclusion, and enough strokes should be applied to achieve more than 80% cell lysis. The nuclei are pelleted by centrifugation at 3300×g for 15 minutes. The supernatant (cytoplasmic extract) is removed. The nuclear pellet is then resuspended in ½ packed nuclei volume of a low-salt buffer (0.02 M Hepes, pH 7.9, 25% volume/volume glycerol, 0.0015 M MgCl₂, 0.02 M KCl, 0.0002 M EDTA, 0.0002 M phenylmethyl sulphonyl fluoride, 0.0005 M dithiothreitol). An equal volume of a high-salt buffer (0.02 M Hepes, pH 7.9, 25% volume/volume glycerol, 0.0015 M MgCl₂, 1.2 M KCl, 0.0002 M EDTA, 0.0002 M phenylmethyl sulphonyl fluoride, 0.0005 M dithiothreitol) is then slowly added to the nuclei suspension with gentle stirring to give a final KCl concentration of roughly 0.3 M. The extraction is allowed to continue for 30 minutes with gentle stirring. After extraction, the nuclei are removed by centrifugation at 25,000×g for 30 minutes. The nuclear extraction is then dialyzed against 50 volumes of a dialysis buffer (0.02 M Hepes, pH 7.9, 20% volume/volume glycerol, 0.1 M KCl, 0.0002 M EDTA, 0.0002 M phenylmethyl sulphonyl fluoride, 0.0005 M dithiothreitol) until the conductivity of the nuclear extract is the same as the dialysis buffer. The nuclear extract is centrifuged once more at 25,000×g for 20 minutes to remove residual debris, and the protein concentration of the supernatant is determined by the micro-BCA assay (Pierce).

The presence of NF-κB in nuclear extract of anti-CD30 stimulated cells can be detected by standard mobility shift DNA-binding assay using the Gel Shift Assay System (Promega, Madison, Wis.). A double stranded oligonucleotide probe containing a consensus NF-κB binding motif with the sequence 5′-AGT TGA GGG GAC TTT CCC AGG C-3′ (SEQ ID NO:33) (Lenardo and Baltimore, 1989, Cell, 58, 227-229) is used as the specific probe to detect NF-κB in nuclear extracts. This probe is phosphorylated by T4 polynucleotide kinase and [α-³²P]ATP. The phosphorylated probe is purified by Sepharose G25 spin columns equilibrated with TE buffer (0.01 M Tris-HCl, pH 8.0, 0.001 M EDTA). Purified probed is then precipitated with ammonium acetate and ethanol and then resuspended in 100 μl of TE buffer. Reaction mixtures containing nuclear extracts from anti-CD30-treated cells and control-treated cells are separately combined with the Gel Shift Binding buffer, water and unlabeled competitor probes according to the manufacturers instruction. An unlabeled oligonucleotide containing the NF-κB consensus and an unlabeled irrelevant oligonucleotide are included in the reaction mixture as the sequence-specific and sequence-nonspecific competitors. After incubation for 10 minutes at room temperature, 1 μl of the ³²P-labeled NF-κB consensus oligonucleotide is added to each reaction. The reactions are allowed to continue for an additional 20 minutes at room temperature. At the end of the incubation, 1 μl of a 10× loading buffer (0.25M Tris-HCl, pH 7.5, 40% volume/volume glycerol, 0.2% bromophenol blue) is added to the reactions. The reactions are then loaded into individual wells of a 6% DNA retardation gel (Invitrogen) and resolved at 100 volt for 90 minutes in 0.5×TBE (0.045M Tris-HCl, 0.045 M boric acid, 0.001M EDTA). After electrophoresis, the gel is covered with plastic wrap and exposed to X-ray film at -70° C. to detect the specific interaction between NF-κB and the oligonucleotide containing the NF-κB binding sequence.

5.5 Nucleic Acids of the Invention

The invention further provides nucleic acids comprising a nucleotide sequence encoding a protein, including but not limited to, a protein of the invention and fragments thereof. Nucleic acids of the invention preferably encode one or more CDRs of antibodies that bind to CD30 and exert cytotoxic or cytostatic effects on HD cells. Exemplary nucleic acids of the invention comprise SEQ ID NO:3, SEQ ID NO:5, SEQ ID NO:7, SEQ ID NO:11, SEQ ID NO:13, SEQ ID NO:15, SEQ ID NO:19, SEQ ID NO:21, SEQ ID NO:23, SEQ ID NO:27, SEQ ID NO:29 or SEQ ID NO:31. Preferred nucleic acids of the invention comprise SEQ ID NO:1, SEQ ID NO:9, SEQ ID NO:17, or SEQ ID NO:25. (See Table 1 at pages 9-10, supra, for identification of the domain of AC10 or HeFi-1 to which these sequence identifiers correspond).

The invention also encompasses nucleic acids that hybridize under stringent, moderate or low stringency hybridization conditions, to nucleic acids of the invention, preferably, nucleic acids encoding an antibody of the invention.

By way of example and not limitation, procedures using such conditions of low stringency for regions of hybridization of over 90 nucleotides are as follows (see also Shilo and Weinberg, 1981, Proc. Natl. Acad. Sci. U.S.A. 78,:6789-6792). Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art (e.g., as employed for cross-species hybridizations).

Also, by way of example and not limitation, procedures using such conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography.

Other conditions of high stringency which may be used depend on the nature of the nucleic acid (e.g. length, GC content, etc.) and the purpose of the hybridization (detection, amplification, etc.) and are well known in the art. For example, stringent hybridization of a nucleic acid of approximately 15-40 bases to a complementary sequence in the polymerase chain reaction (PCR) is done under the following conditions: a salt concentration of 50 mM KCl, a buffer concentration of 10 mM Tris-HCl, a Mg²⁺ concentration of 1.5 mM, a pH of 7-7.5 and an annealing temperature of 55-60° C.

In another specific embodiment, a nucleic acid which is hybridizable to a nucleic acid of the invention acid, or its complement, under conditions of moderate stringency is provided. Selection of appropriate conditions for such stringencies is well known in the art (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, © 1987-1997, Current Protocols, © 1994-1997 John Wiley and Sons, Inc.).

The nucleic acids of the invention may be obtained, and the nucleotide sequence of the nucleic acids determined, by any method known in the art. For example, if the nucleotide sequence of the protein is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the protein, annealing and ligating of those oligonucleotides, and then amplification of the ligated oligonucleotides by PCR.

Alternatively, a nucleic acid encoding a protein of the invention may be generated from nucleic acid from a suitable source. If a clone containing a nucleic acid encoding a particular protein is not available, but the sequence of the protein molecule is known, a nucleic acid encoding the protein may be chemically synthesized or obtained from a suitable source (e.g., a cDNA library such as an antibody cDNA library or a cDNA library generated from, or nucleic acid, preferably poly A+ RNA, isolated from, any tissue or cells expressing the protein. If the protein is an antibody, the library source can be hybridoma cells selected to express the antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the protein. Amplified nucleic acids generated by PCR may then be cloned into replicable cloning vectors using any method well known in the art.

Once the nucleotide sequence and corresponding amino acid sequence of the antibody is determined, the nucleotide sequence of the protein may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties ), to generate antibodies having a different amino acid sequence, for example to create amino acid substitutions, deletions, and/or insertions.

In a specific embodiment, the protein is an antibody, and the amino acid sequence of the heavy and/or light chain variable domains may be inspected to identify the sequences of the CDRs by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and are preferably human framework regions (see, e.g., Chothia et al., 1998, J. Mol. Biol. 278:457-479 for a listing of human framework regions). The nucleic acid generated by the combination of the framework regions and CDRs encodes an antibody that specifically binds CD30 and exerts a cytostatic and/or cytotoxic effect on HD cells. Preferably, as discussed supra, one or more amino acid substitutions may be made within the framework regions, and, preferably, the amino acid substitutions improve binding of the antibody to CD30 and/or to enhance the cytostatic and/or cytotoxic effect of the antibody. Additionally, such methods may be used to make amino acid substitutions or deletions of one or more variable region cysteine residues participating in an intrachain disulfide bond to generate antibody molecules lacking one or more intrachain disulfide bonds. Other alterations to the nucleic acid are encompassed by the present invention and within the skill of the art.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54) can be adapted to produce single chain antibodies. Single chain antibodies are formed by linking the heavy and light chain fragments of the Fv region via an amino acid bridge, resulting in a single chain protein. Techniques for the assembly of functional Fv fragments in E. coli may also be used (Skerra et al., 1988, Science 242:1038-1041).

5.6 Sequences Related to AC10 and HeFi-1

The present invention further encompasses proteins and nucleic acids comprising a region of homology to CDRs of AC10 and HeFi-1, or the coding regions therefor, respectively. In various embodiments, the region of homology is characterized by at least 50%, at least 55%, at least 60%, at least 65%, at least 70%, at least 75%, at least 80%, at least 85%, at least 90%, at least 95% or at least 98% identity with the corresponding region of AC10 or HeFi-1.

In one embodiment, the present invention provides a protein with a region of homology to a CDR of HeFi-1 (SEQ ID NO:20, SEQ ID NO:22; SEQ ID NO:24; SEQ ID NO:28, SEQ ID NO:30 or SEQ ID NO:32). In another embodiment, the present invention provides a protein with a region of homology to a CDR of AC10 (SEQ ID NO:4; SEQ ID NO:6; SEQ ID NO:8; SEQ ID NO:12; SEQ ID NO:14; or SEQ ID NO:16).

In another embodiment, the present invention provides a nucleic acid with a region of homology to a CDR coding region of HeFi-1 (SEQ ID NO:19, SEQ ID NO:21, SEQ ID NO:23, SEQ ID NO:27, SEQ ID NO:29 or SEQ ID NO:31). In yet another embodiment, the present invention provides a nucleic acid with a region of homology to a CDR coding region of AC10 (SEQ ID NO:3, SEQ ID NO:5, SEQ ID NO:7, SEQ ID NO:11, SEQ ID NO:13, SEQ ID NO:15).

The present invention further encompasses proteins and nucleic acids comprising a region of homology to the variable regions of AC10 and HeFi-1, or the coding region therefor, respectively. In various embodiments, the region of homology is characterized by at least 50%, at least 55%, at least 60%, at least 65%, at least 70%, at least 75%, at least 80%, at least 85%, at least 90%, at least 95% or at least 98% identity with the corresponding region of AC10 or HeFi-1.

In one embodiment, the present invention provides a protein with a region of homology to a variable region of HeFi-1 (SEQ ID NO:18 or SEQ ID NO:26). In another embodiment, the present invention provides a protein with a region of homology to a variable region of AC10 (SEQ ID NO:2 or SEQ ID NO:10).

In one embodiment, the present invention provides a nucleic acid with a region of homology to a variable region coding region of HeFi-1 (SEQ ID NO:17 or SEQ ID NO:25). In another embodiment, the present invention provides a nucleic with a region of homology to a variable region coding region of AC10 (SEQ ID NO:1 or SEQ ID NO:9).

To determine the percent identity of two amino acid sequences or of two nucleic acids, e.g. between the sequences of an AC10 or HeFi-1 variable region and sequences from other proteins with regions of homology to the AC10 or HeFi-1 variable region, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid encoding a SCA-1 modifier protein. BLAST protein searches can be performed with the XBLAST program, score=50, wordlength=3 to obtain amino acid sequences homologous to a SCA-1 modifier protein. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci., 10:3-5; and FASTA described in Pearson and Lipman, 1988, Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference.

Alternatively, protein sequence alignment may be carried out using the CLUSTAL W algorithm, as described by Higgins et al., 1996, Methods Enzymol. 266:383-402.

The percent identity between two sequences can be determined using techniques similar to those described above, with or without allowing gaps. In calculating percent identity, only exact matches are counted.

5.7 Methods of Producing the Proteins of the Invention

The proteins, including antibodies, of the invention can be produced by any method known in the art for the synthesis of proteins, in particular, by chemical synthesis or preferably, by recombinant expression techniques.

Recombinant expression of a protein of the invention, including a fragment, derivative or analog thereof, (e.g., a heavy or light chain of an antibody of the invention) requires construction of an expression vector containing a nucleic acid that encodes the protein. Once a nucleic acid encoding a protein of the invention has been obtained, the vector for the production of the protein molecule may be produced by recombinant DNA technology using techniques well known in the art. Thus, methods for preparing a protein by expressing a nucleic acid containing nucleotide sequence encoding said protein are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a protein of the invention operably linked to a promoter. Wherein the protein is an antibody, the nucleotide sequence may encode a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce a protein of the invention. Thus, the invention encompasses host cells containing a nucleic acid encoding a protein of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the proteins molecules of the invention. Such host-expression systems represent vehicles by which the coding sequences of interest may be produced and subsequently purified, but also represent cells which may, when transformed or transfected with the appropriate nucleotide coding sequences, express a protein of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecules, are used for the expression of a recombinant protein of the invention. For example, mammalian cells such as Chinese hamster ovary cells (CHO), in conjunction with a vector such as the major intermediate early gene promoter element from human cytomegalovirus is an effective expression system for proteins of the invention (Foecking et al., 1986, Gene 45:101; Cockett et al., 1990, Bio/Technology 8:2).

In bacterial systems, a number of expression vectors may be advantageously selected depending upon the use intended for the folding and post-translation modification requirements protein being expressed. Where possible, when a large quantity of such a protein is to be produced, for the generation of pharmaceutical compositions comprising a protein of the invention, vectors which direct the expression of high levels of fusion protein products that are readily purified may be desirable. Such vectors include, but are not limited, to the E. coli expression vector pUR278 (Ruther et al., 1983, EMBO 1. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express fusion proteins with glutathione S-transferase (GST). In general, such fusion proteins are soluble and can easily be purified from lysed cells by adsorption and binding to matrix glutathioneagarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the coding sequence of the protein of the invention may be ligated to an adenovirus transcription/translation control complex, e.g., the late promoter and tripartite leader sequence. This chimeric gene may then be inserted in the adenovirus genome by in vitro or in vivo recombination. Insertion in a non- essential region of the viral genome (e.g., region E1 or E3) will result in a recombinant virus that is viable and capable of expressing the protein of the invention in infected hosts. (See, e.g., Logan & Shenk, 1984, Proc. Natl. Acad. Sci. USA 8 1:355-359). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein of the invention. Different host cells have characteristic and specific mechanisms for the post-translational processing and modification of proteins and gene products. Appropriate cell lines or host systems can be chosen to ensure the correct modification and processing of the foreign protein expressed. To this,end, eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK, 293, 3T3, and W138.

For long-term, high-yield production of recombinant proteins, stable expression is preferred. For example, cell lines which stably express the protein of the invention may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the protein of the invention.

A number of selection systems may be used, including but not limited to the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt- cells, respectively. Also, antimetabolite resistance can be used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95 ; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of a protein of the invention can be increased by vector amplification (for a review, see Bebbington and Hentschel, “The Use of Vectors Based on Gene Amplification for the Expression of Cloned Genes in Mammalian Cells in DNY Cloning”, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the protein of the invention will also increase (Crouse et al., 1983, Mol. Cell. Biol. 3:257).

Wherein the protein of the invention is an antibody, the host cell may be co-transfected with two expression vectors of the invention, the first vector encoding a heavy chain derived protein and the second vector encoding a light chain derived protein. The two vectors may contain identical selectable markers which enable equal expression of heavy and light chain proteins. Alternatively, a single vector may be used which encodes, and is capable of expressing, both heavy and light chain proteins. In such situations, the light chain should be placed before the heavy chain to avoid an excess of toxic free heavy chain (Proudfoot, 1986, Nature 322:52 (1986); Kohler, 1980, Proc. Natl. Acad. Sci. USA 77:2 197). The coding sequences for the heavy and light chains may comprise cDNA or genomic DNA.

Once a protein molecule of the invention has been produced by an animal, chemically synthesized, or recombinantly expressed, it may be purified by any method known in the art for purification of proteins, for example, by chromatography (e.g., ion exchange; affinity, particularly by affinity for the specific antigen, Protein A (for antibody molecules, or affinity for a heterologous fusion partner wherein the protein is a fusion protein; and sizing column chromatography), centrifugation, differential solubility, or by any other standard technique for the purification of proteins.

The present invention encompasses CD3-binding proteins recombinantly fused or chemically conjugated (including both covalent and non-covalent conjugation) to heterologous proteins (of preferably at least 10, 20, 30, 40, 50, 60, 70, 80, 90 or at least 100 amino acids) of the present invention to generate fusion proteins. The fusion does not necessarily need to be direct, but may occur through linker sequences.

The present invention further includes compositions comprising proteins of the invention fused or conjugated to antibody domains other than the variable regions. For example, the proteins of the invention may be fused or conjugated to an antibody Fc region, or portion thereof. The antibody portion fused to a protein of the invention may comprise the constant region, hinge region, CH 1 domain, CR2 domain, and CH3 domain or any combination of whole domains or portions thereof. The proteins may also be fused or conjugated to the above antibody portions to form multimers. For example, Fc portions fused to the proteins of the invention can form dimers through disulfide bonding between the Fc portions. Higher multimeric forms can be made by fusing the proteins to portions of IgA and IgM. Methods for fusing or conjugating the proteins of the invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 9 1/06570; Ashkenazi et al., 1991, Proc. Nat. Acad. Sci. USA 88:10535-10539; Zheng et al., 1995, J. Immunol. 154:5590-5600; and Vil et al., 1992, Proc. Natl. Acad. Sci. USA 89:11337-11341 (said references incorporated by reference in their entireties).

5.8 Conjugates and Fusion Proteins

As discussed, supra, the proteins of the invention encompass proteins that bind to CD30 and exert a cytostatic and/or cytotoxic effect on HD cells, and that are further fused or conjugated to heterologous proteins or cytotoxic agents.

The present invention thus provides for treatment of Hodgkin's Disease by administration of a protein or nucleic acid of the invention. Proteins of the invention include but are not limited to: AC10 and HeFi-1 proteins, antibodies and analogs and derivatives thereof (e.g., as described herein above); the nucleic acids of the invention include but are not limited to nucleic acids encoding such AC10 and HeFi-1 proteins, antibodies and analogs or derivatives (e.g., as described herein above).

In certain embodiments of the invention, a protein or nucleic acid of the invention may be chemically modified to improve its cytotoxic and/or cytostatic properties. For example, a protein of the invention can be administered as a conjugate. Particularly suitable moieties for conjugation to proteins of the invention are chemotherapeutic agents, pro-drug converting enzymes, radioactive isotopes or compounds, or toxins. Alternatively, a nucleic acid of the invention may be modified to functionally couple the coding sequence of a pro-drug converting enzyme with the coding sequence of a protein of the invention, such that a fusion protein comprising the functionally active pro-drug converting enzyme and protein of the invention is expressed in the subject upon administration of the nucleic acid in accordance with the gene therapy methods described in Section 5.7, infra.

In one embodiment, a protein of the invention is fused to a marker sequence, such as a peptide, to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., 1984, Cell 37:767) and the “flag” tag. Such fusion proteins can be generated by standard recombinant methods known to those of skill in the art.

In another embodiment, the proteins of the invention are fused or conjugated to a therapeutic agent. For example, a protein of the invention may be conjugated to a cytotoxic agent such as a chemotherapeutic agent, a toxin (e.g., a cytostatic or cytocidal agent), or a radionuclide (e.g., alpha-emitters such as, for example, ²¹²Bi, ²¹¹At, or beta-emitters such as, for example, ¹³¹I, ⁹⁰Y, or ⁶⁷Cu).

Drugs such as methotrexate (Endo et al., 1987, Cancer Research 47:1076-1080), daunomycin (Gallego et al., 1984, Int. J. Cancer. 33:737-744), mitomycin C (MMC) (Ohkawa et al., 1986, Cancer Immunol. Immunother. 23:81-86) and vinca alkaloids (Rowland et al., 1986, Cancer Immunol Immunother. 21:183-187) have been attached to antibodies and the derived conjugates have been investigated for anti-tumor activities. Care should be taken in the generation of chemotherapeutic agent conjugates to ensure that the activity of the drug and/or protein does not diminish as a result of the conjugation process.

Examples of chemotherapeutic agents include the following non-mutually exclusive classes of chemotherapeutic agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids. Examples of individual chemotherapeutics that can be conjugated to a nucleic acid or protein of the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26. In a preferred embodiment, the chemotherapeutic agent is auristatin E. In a more preferred embodiment, the chemotherapeutic agent is the auristatin E derivative AEB (as described in U.S. application Ser. No. 09/845,786 filed Apr. 30, 2001, which is incorporated by reference here in its entirety).

The conjugates of the invention used for enhancing the therapeutic effect of the protein of the invention include non-classical therapeutic agents such as toxins. Such toxins include, for example, abrin, ricin A, pseudomonas exotoxin, or diphtheria toxin.

Techniques for conjugating such therapeutic moieties to proteins, and in particular to antibodies, are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc., 1985); Hellstrom et al., “Antibodies For Drug Delivery”, in Controlled Drug Delivery (2nd ed.), Robinson et al. (eds.), pp. 623-53 (Marcel Dekker, Inc., 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody of the invention can be conjugated to a second antibody to form an antibody heteroconjugate as described by Segal in U.S. Pat. No. 4,676,980, which is incorporated herein by reference in its entirety.

As discussed above, in certain embodiments of the invention, a protein of the invention can be co-administered with a pro-drug converting enzyme. The pro-drug converting enzyme can be expressed as a fusion protein with or conjugated to a protein of the invention. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

5.9 Anti-CD30 Antibody-Drug Conjugates

The present invention encompasses the use of anti-CD30 antibody-drug conjugates (anti-CD30 ADCs) for the treatment or prevention of an immunological disorder. The ADCs of the invention are tailored to produce clinically beneficial cytotoxic or cytostatic effects on CD30-expressing cells when administered to a patient with an immune disorder involving CD30-expressing cells, preferably when administered alone but also in combination with other therapeutic agents.

Techniques for conjugating such drugs to proteins, and in particular to antibodies, are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc., 1985); Hellstrom et al., “Antibodies For Drug Delivery”, in Controlled Drug Delivery (2nd ed.), Robinson et al. (eds.), pp. 623-53 (Marcel Dekker, Inc., 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Because in many of the disease states that are encompassed by the treatment methods of the present invention a significant amount of soluble CD30 is shed from the activated lymphocytes, it is preferable when using an anti-CD30 antibody that is conjugated to a drug (e.g., a cytotoxic agent or an immunosuppressive agent) or prodrug converting enzyme that the drug or prodrug converting enzyme is active in the vicinity of the activated lymphocytes rather than any place in the body that soluble CD30 may be found.

Two approaches may be taken to minimize drug activity outside the activated lymphocytes that are targeted by the anti-CD30 antibodies of the invention: first, an antibody that binds to cell membrane but not soluble CD30 may be used, so that the drug, including drug produced by the actions of the prodrug converting enzyme, is concentrated at the cell surface of the activated lymphocyte. A more preferred approach for minimizing the activity of drugs bound to the antibodies of the invention is to conjugate the drugs in a manner that would reduce their activity unless they are hydrolyzed or cleaved off the antibody. Such methods would employ attaching the drug to the antibodies with linkers that are sensitive to the environment at the cell surface of the activated lymphocyte (e.g., the activity of a protease that is present at the cell surface of the activated lymphocyte) or to the environment inside the activated lymphocyte the conjugate encounters when it is taken up by the activated lymphocyte (e.g., in the endosomal or, for example by virtue of pH sensitivity or protease sensitivity, in the lysosomal environment).

In one embodiment, the linker is an acid- labile hydrazone or hydrazide group that is hydrolyzed in the lysosome (see, e.g., U.S. Pat. No. 5,622,929) In alternative embodiments, drugs can be appended to anti-CD30 antibodies through other acid-labile linkers, such as cis-aconitic amides, orthoesters, acetals and ketals (Dubowchik and Walker, 1999, Pharm. Therapeutics 83:67-123; Neville et al., 1989, Biol. Chem. 264:14653-14661). Such linkers are relatively stable under neutral pH conditions, such as those in the blood, but are unstable at below pH 5, the approximate pH of the lysosome.

In other embodiments, drugs are attached to the anti-CD30 antibodies of the invention using peptide spacers that are cleaved by intracellular proteases. Target enzymes include cathepsins B and D and plasmin, all of which are known to hydrolyze dipeptide drug derivatives resulting in the release of active drug inside target cells (Dubowchik and Walker, 1999, Pharm. Therapeutics 83:67-123). The advantage of using intracellular proteolytic drug release is that the drug is highly attenuated when conjugated and the serum stabilities of the conjugates can be extraordinarily high.

In yet other embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

The drugs used for conjugation to the anti-CD30 antibodies of the present invention can include conventional chemotherapeutics, such as doxorubicin, paclitaxel, melphalan, vinca alkaloids, methotrexate, mitomycin C, etoposide, and others. In addition, potent agents such CC-1065 analogues, calichiamicin, maytansine, analogues of dolastatin 10, rhizoxin, and palytoxin can be linked to the anti-CD30 antibodies using the conditionally stable linkers to form potent immunoconjugates. Examples of other suitable drugs for conjugation to the anti-CD30 antibodies of the present invention are provided in Section 5.12.1, infra.

5.9.1 Linkers

As discussed above in Section 5.6, ADCs are generally made by conjugating a drug to an antibody through a linker. Thus, a majority of the ADCs of the present invention, which comprise an anti-CD30 antibody and a high potency drug and/or an intemalization-promoting drug, further comprise a linker. Any linker that is known in the art may be used in the ADCs of the present invention, e.g., bifunctional agents (such as dialdehydes or imidoesters) or branched hydrazone linkers (see, e.g., U.S. Pat. No. 5,824,805, which is incorporated by reference herein in its entirety).

In certain, non-limiting, embodiments of the invention, the linker region between the drug moiety and the antibody moiety of the anti-CD30 ADC is cleavable or hydrolyzable under certain conditions, wherein cleavage or hydrolysis of the linker releases the drug moiety from the antibody moiety. Preferably, the linker is sensitive to cleavage or hydrolysis under intracellular conditions.

In a preferred embodiment, the linker region between the drug moiety and the antibody moiety of the anti-CD30 ADC is hydrolyzable if the pH changes by a certain value or exceeds a certain value. In a particularly preferred embodiment of the invention, the linker is hydrolyzable in the milieu of the lysosome, e.g., under acidic conditions (i.e., a pH of around 5-5.5 or less). In other embodiments, the linker is a peptidyl linker that is cleaved by a peptidase or protease enzyme, including but not limited to a lysosomal protease enzyme, a membrane-associated protease, an intracellular protease, or an endosomal protease. Preferably, the linker is at least two amino acids long, more preferably at least three amino acids long. Peptidyl linkers that are cleavable by enzymes that are present in CD30-expressing cancers are preferred. For example, a peptidyl linker that is cleavable by cathepsin-B (e.g., a Gly-Phe-Leu-Gly linker), a thiol-dependent protease that is highly expressed in cancerous tissue, can be used. Other such linkers are described, e.g., in U.S. Pat. No. 6,214,345, which is incorporated by reference in its entirety herein.

In other, non-mutually exclusive embodiments of the invention, the linker by which the anti-CD30 antibody and the drug of an ADC of the invention are conjugated promotes cellular internalization. In certain embodiments, the linker-drug moiety of the ADC promotes cellular internalization. In certain embodiments, the linker is chosen such that the structure of the entire ADC promotes cellular internalization.

In a specific embodiment of the invention, derivatives of valine-citrulline are used as linker (val-cit linker). The synthesis of doxorubicin with the val-cit linker have been previously described (U.S. Pat. No. 6,214,345 to Dubowchik and Firestone, which is incorporated by reference herein in its entirety).

In another specific embodiment, the linker is a phe-lys linker.

In another specific embodiment, the linker is a thioether linker (see, e.g., U.S. Pat. No. 5,622,929 to Willner et al., which is incorporated by reference herein in its entirety).

In yet another specific embodiment, the linker is a hydrazone linker (see, e.g., U.S. Pat. No. 5,122,368 to Greenfield et al and U.S. Pat. No. 5,824,805 to King et al., which are incorporated by reference herein in their entireties).

In yet other specific embodiments, the linker is a disulfide linker. A variety of disulfide linkers are known in the art, including but not limited to those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene). SPDB and SMPT (see, e.g., Thorpe et al., 1987, Cancer Res., 47:5924-5931; Wawrzynczak et al.,1987, In Immunoconjugates: Antibody Conjugates in Radioimagery and Therapy of Cancer, ed. C. W. Vogel, Oxford U. Press, pp. 28-55; see also U.S. Pat. No. 4,880,935 to Thorpe et al., which is incorporated by reference herein in its entirety).

A variety of linkers that can be used with the compositions and methods of the present invention are described in U.S. provisional application No. 60/400,403, entitled “Drug Conjugates and their use for treating cancer, an autoimmune disease or an infectious disease”, by Inventors: Peter D. Senter, Svetlana Doronina and Brian E. Toki, submitted on Jul. 31, 2002, which is incorporated by reference in its entirety herein.

In yet other embodiments of the present invention, the linker unit of an anti-CD30 antibody-linker-drug conjugate (anti-CD30 ADC) links the cytotoxic or cytostatic agent (drug unit; —D) and the anti-CD30 antibody unit (—A). As used herein the term anti-CD30 ADC encompasses anti-CD30 antibody drug conjugates with and without a linker unit. The linker unit has the general formula:

—Tₐ—W_{w}—Y_{y}

wherein:

—T— is a stretcher unit;

a is 0 or 1;

each —W— is independently an amino acid unit;

w is independently an integer ranging from 2 to 12;

—Y— is a spacer unit; and

y is 0, 1 or 2.

5.9.2 The Stretcher Unit

The stretcher unit (—T—), when present, links the anti-CD30 antibody unit to an amino acid unit (—W—). Useful functional groups that can be present on an anti-CD30 antibody, either naturally or via chemical manipulation include, but are not limited to, sulfhydryl, amino, hydroxyl, the anomeric hydroxyl group of a carbohydrate, and carboxyl. Preferred functional groups are sulfhydryl and amino. Sulfhydryl groups can be generated by reduction of the intramolecular disulfide bonds of an anti-CD30 antibody. Alternatively, sulthydryl groups can be generated by reaction of an amino group of a lysine moiety of an anti-CD30 antibody with 2-iminothiolane (Traut's reagent) or other sulfhydryl generating reagents. In specific embodiments, the anti-CD30 antibody is a recombinant antibody and is engineered to carry one or more lysines. In other embodiments, the recombinant anti-CD30 antibody is engineered to carry additional sulfhydryl groups, e.g., additional cysteines.

In certain specific embodiments, the stretcher unit forms a bond with a sulfur atom of the anti-CD30 antibody unit. The sulfur atom can be derived from a sulfhydryl (—SH) group of a reduced anti-CD30 antibody (A). Representative stretcher units of these embodiments are depicted within the square brackets of Formulas (Ia) and (Ib; see infra), wherein A—, —W—, —Y—, —D, w and y are as defined above and R¹ is selected from —C₁-C₁₀ alkylene-, —C₃-C8 carbocyclo-, —O—(C₁-C₈alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —(CH₂CH₂O)ᵣ—, and —(CH₂CH₂O)ᵣ—CH₂—; and r is an integer ranging from 1-10.

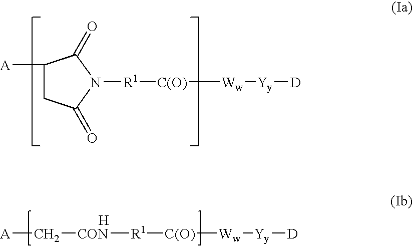

An illustrative stretcher unit is that of formula (Ia) where R¹ is —(CH₂)₅—:

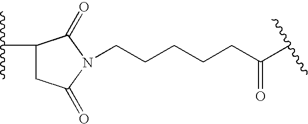

Another illustrative stretcher unit is that of formula (Ia) where R¹ is —(CH₂CH₂O)ᵣ—CH₂—; and r is 2:

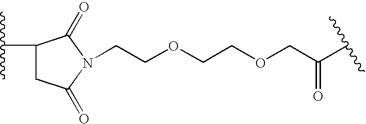

Still another illustrative stretcher unit is that of formula (Ib) where R¹ is —(CH₂)₅—:

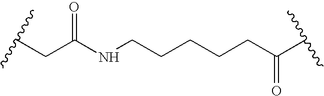

In certain other specific embodiments, the stretcher unit is linked to the anti-CD30 antibody unit (A) via a disulfide bond between a sulfuir atom of the anti-CD30 antibody unit and a sulfur atom of the stretcher unit. A representative stretcher unit of this embodiment is depicted within the square brackets of Formula (II), wherein R¹, A—, —W—, —Y—, —D, w and y are as defined above.

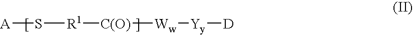

In even other specific embodiments, the reactive group of the stretcher contains a reactive site that can be reactive to an amino group of an anti-CD30 antibody. The amino group can be that of an arginine or a lysine. Suitable amine reactive sites include, but are not limited to, activated esters such as succinimide esters, 4-nitrophenyl esters, pentafluorophenyl esters, anhydrides, acid chlorides, sulfonyl chlorides, isocyanates and isothiocyanates. Representative stretcher units of these embodiments are depicted within the square brackets of Formulas (IIIa) and (IIIb), wherein R¹, A—, —W—, —Y—, —D, w and y are as defined above;

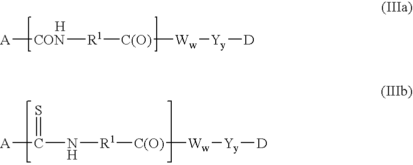

In yet another aspect of the invention, the reactive function of the stretcher contains a reactive site that is reactive to a modified carbohydrate group that can be present on an anti-CD30 antibody. In a specific embodiment, the anti-CD30 antibody is glycosylated enzymatically to provide a carbohydrate moiety. The carbohydrate may be mildly oxidized with a reagent such as sodium periodate and the resulting carbonyl unit of the oxidized carbohydrate can be condensed with a stretcher that contains a functionality such as a hydrazide, an oxime, a reactive amine, a hydrazine, a thiosemicarbazone, a hydrazine carboxylate, and an arylhydrazide such as those described by Kaneko, T. et al. Bioconjugate Chem 1991, 2, 133-41. Representative stretcher units of this embodiment are depicted within the square brackets of Formulas (IVa)-(IVc), wherein R¹, A—, —W—, —Y—, —D, w and y are as defined above.

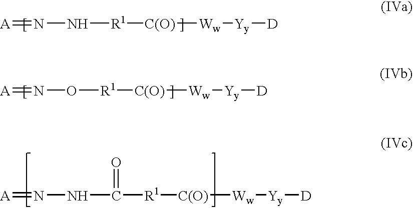

5.9.3 The Amino Acid Unit

The amino acid unit (—W—) links the stretcher unit (—T—) to the Spacer unit (—Y—) if the Spacer unit is present, and links the stretcher unit to the cytotoxic or cytostatic agent (Drug unit; D) if the spacer unit is absent.

—W_{w}— is a dipeptide, tripeptide, tetrapeptide, pentapeptide, hexapeptide, heptapeptide, octapeptide, nonapeptide, decapeptide, undecapeptide or dodecapeptide unit. Each —W— unit independently has the formula denoted below in the square brackets, and w is an integer ranging from 2 to 12:

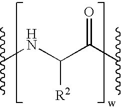

wherein R² is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

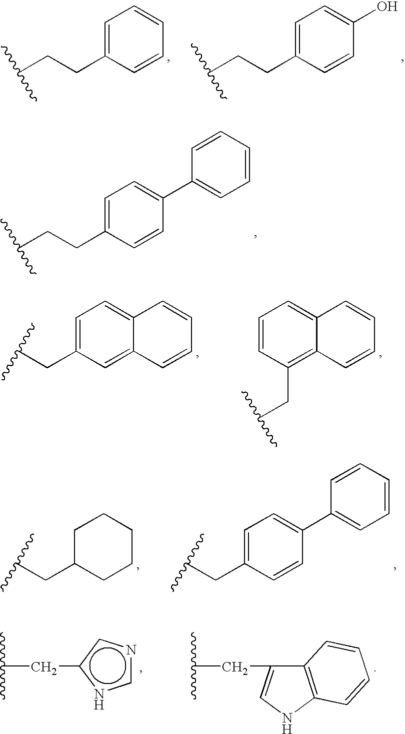

The amino acid unit of the linker unit can be enzymatically cleaved by an enzyme including, but not limited to, a tumor-associated protease to liberate the drug unit (—D) which is protonated in vivo upon release to provide a cytotoxic drug (D). Illustrative W_{w} units are represented by formulas (V)-(VII):

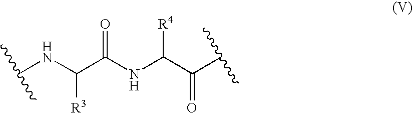

wherein R³ and R⁴ are as follows:

| R³ | R⁴ |
|---|---|
| Benzyl | CH₂)₄NH₂; |
| Methyl | (CH₂)₄NH₂; |
| Isopropyl | (CH₂)₄NH₂; |
| Isopropyl | (CH₂)₃NHCONH₂; |
| Benzyl | (CH₂)₃NHCONH₂; |
| Isobutyl | (CH₂)₃NHCONH₂; |
| sec-butyl | (CH₂)₃NHCONH₂; |
| 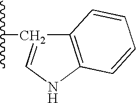 | (CH₂)₃NHCONH₂; |
| Benzyl | methyl; and |
| Benzyl | (CH₂)₃NHC(═NH)NH₂; |

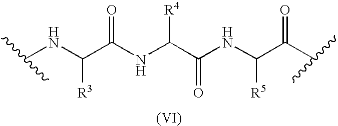

wherein R³, R⁴ and R⁵ are as follows:

| R³ | R⁴ | R⁵ |
|---|---|---|
| benzyl | Benzyl | (CH₂)₄NH₂; |
| isopropyl | Benzyl | (CH₂)₄NH₂; and |
| H | Benzyl | (CH₂)₄NH₂; |

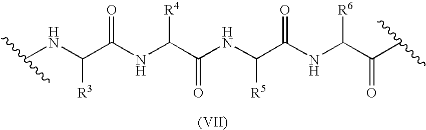

wherein R³, R⁴, R⁵ and R⁶ are as follows:

| R³ | R⁴ | R⁵ | R⁶ |
|---|---|---|---|
| H | Benzyl | Isobutyl | H; and |
| methyl | isobutyl | Methyl | isobutyl. |

Preferred amino acid units include, but are not limited to, units of formula (V) where: R³ is benzyl and R⁴ is —(CH₂)₄NH₂; R³ is isopropyl and R⁴ is —(CH₂)₄NH₂; R³ is isopropyl and R⁴ is —(CH₂)₃NHCONH₂. Another preferred amino acid unit is a unit of formula (VI), where: R³ is benzyl, R⁴ is benzyl, and R⁵ is —(CH₂)₄NH₂.

—W_{w}— units useful in the present invention can be designed and optimized in their selectivity for enzymatic cleavage by a particular tumor-associated protease. The preferred —W_{w}— units are those whose cleavage is catalyzed by the proteases, cathepsin B, C and D, and plasmin.

In one embodiment, —W_{w}— is a dipeptide, tripeptide or tetrapeptide unit.

Where R², R³, R⁴, R⁵ or R⁶ is other than hydrogen, the carbon atom to which R², R³, R⁴, R⁵ or R⁶ is attached is chiral.

Each carbon atom to which R², R³, R⁴, R⁵ or R⁶ is attached is independently in the (S) or (R) configuration.

In a preferred embodiment, the amino acid unit is a phenylalanine-lysine dipeptide (phe-lys or FK linker). In antother preferred embodiment, the amino acid unit is a valine-citrulline dipeptide (val-cit or VC linker).

5.9.4 The Spacer Unit

The spacer unit (—Y—), when present, links an amino acid unit to the drug unit. Spacer units are of two general types: self-immolative and non self-immolative. A non self-immolative spacer unit is one in which part or all of the spacer unit remains bound to the drug unit after enzymatic cleavage of an amino acid unit from the anti-CD30 antibody-linker-drug conjugate or the drug-linker compound. Examples of a non self-immolative spacer unit include, but are not limited to a (glycine-glycine) spacer unit and a glycine spacer unit (both depicted in Scheme 1). When an anti-CD30 antibody-linker-drug conjugate of the invention containing a glycine-glycine spacer unit or a glycine spacer unit undergoes enzymatic cleavage via a tumor-cell associated-protease, a cancer-cell-associated protease or a lymphocyte-associated protease, a glycine-glycine-drug moiety or a glycine-drug moiety is cleaved from A—T—W_{w}—. To liberate the drug, an independent hydrolysis reaction should take place within the target cell to cleave the glycine-drug unit bond.

In a preferred embodiment, —Y_{y}— is a p-aminobenzyl ether which can be substituted with Qₘ where Q is is —C₁-C₈ alkyl, —C₁-C₈ alkoxy, -halogen,-nitro or -cyano; and m is an integer ranging from 0-4.

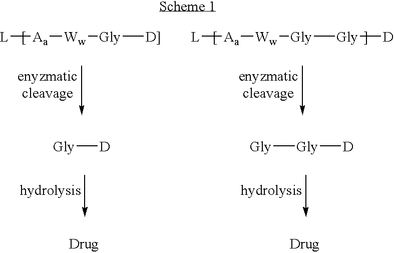

In one embodiment, a non self-irnmolative spacer unit (—Y—) is -Gly-Gly-.

In another embodiment, a non self-immolative the spacer unit (—Y—) is -Gly-.

In one embodiment, the drug-linker compound or an anti-CD30 antibody-linker-drug conjugate lacks a spacer unit (y=0).

Alternatively, an anti-CD30 antibody-linker-drug conjugate of the invention containing a self-immolative spacer unit can release the drug (D) without the need for a separate hydrolysis step. In these embodiments, —Y— is a p-aminobenzyl alcohol (PAB) unit that is linked to —W_{w}— via the nitrogen atom of the PAB group, and connected directly to —D via a carbonate, carbamate or ether group (Scheme 2 and Scheme 3).

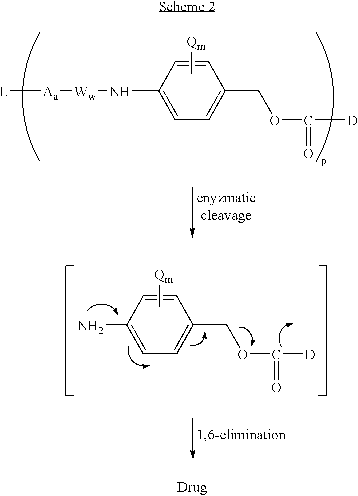

where Q is —C₁-C₈ alkyl, —C₁-C₈ alkoxy, -halogen, -nitro or -cyano; m is an integer ranging from 0-4; and p is an integer ranging from 1-20.

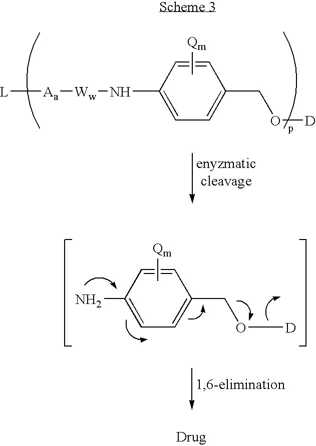

where Q is —C₁-C₈ alkyl, —C₁-C₈ alkoxy, -halogen,-nitro or -cyano; m is an integer ranging from 0-4; and p is an integer ranging from 1-20.

Other examples of self-immolative spacers include, but are not limited to, aromatic compounds that are electronically equivalent to the PAB group such a 2-aminoimidazol-5-methanol derivatives (see Hay et al., Bioorg. Med. Chem. Lett., 1999, 9, 2237 for examples) and ortho or para-aminobenzylacetals. Spacers can be used that undergo facile cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867). Elimination of amine-containing drugs that are substituted at the a-position of glycine (Kingsbury, et al., J. Med. Chem., 1984, 27, 1447) are also examples of self-immolative spacer strategies that can be applied to the anti-CD30 antibody-linker-drug conjugates of the invention.

In an alternate embodiment, the spacer unit is a branched bis(hydroxymethyl)styrene (BHMS) unit (Scheme 4), which can be used to incorporate additional drugs.

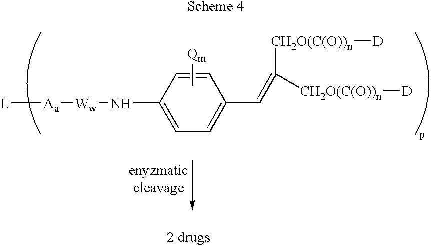

where Q is —C₁-C₈ alkyl, —C₁-C₈ alkoxy, -halogen, -nitro or -cyano; m is an integer ranging from 0-4; n is 0 or 1; and p is an integer raging from 1-20.

In one embodiment, the two —D moieties are the same.

In another embodiment, the two —D moieties are different.

Preferred spacer units (—Y_{y}—) are represented by Formulas (VIII)-(X):

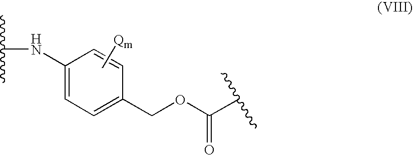

where Q is C₁-C₈ alkyl, C₁-C₈ alkoxy, halogen, nitro or cyano; and m is an integer ranging from 0-4;

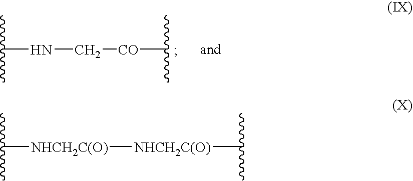

5.10 Drugs

The present invention encompasses the use of anti-CD30 ADCs for the treatment or prevention of an immunological disorder. As used herein, the term “drug” or “cytotoxic agent,” where employed in the context of an anti-CD30 ADC of the invention, does not include radioisotopes. Otherwise, any drug that is known to the skilled artisan can be used in connection with the ADCs of the present invention.

The drugs used for conjugation to the anti-CD30 antibodies of the present invention can include conventional chemotherapeutics, such as doxorubicin, paclitaxel, melphalan, vinca alkaloids, methotrexate, mitomycin C, etoposide, and others. In addition, potent agents such CC-1065 analogues, calichiamicin, maytansine, analogues of dolastatin 10, rhizoxin, and palytoxin can be linked to the anti-CD30 antibodies using the conditionally stable linkers to form potent immunoconjugates. Examples of other suitable drugs for conjugation to the anti-CD30 antibodies of the present invention are provided in Section 5.12.1 below.

In certain embodiments, the ADCs of the invention comprise drugs that are at least 40-fold more potent than doxorubicin on CD30-expressing cells. Such drugs include, but are not limited to: DNA minor groove binders, including enediynes and lexitropsins, duocarmycins, taxanes (including paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epithilone A and B, estramustine, cryptophysins, cemadotin, maytansinoids, dolastatins, e.g., auristatin E, dolastatin 10, MMAE, discodermolide, eleutherobin, and mitoxantrone.

In certain specific embodiments, an anti-CD30 ADC of the invention comprises an enediyne moiety. In a specific embodiment, the enediyne moiety is calicheamicin. Enediyne compounds cleave double stranded DNA by generating a diradical via Bergman cyclization.

A variety of cytotoxic and cytostatic agents that can be used with the compositions and methods of the present invention are described in U.S. provisional application No. 60/400,403, entitled “Drug Conjugates and their use for treating cancer, an autoimmune disease or an infectious disease”, by Inventors: Peter D. Senter, Svetlana Doronina and Brian E. Toki, filed on Jul. 31, 2002, which is incorporated by reference in its entirety herein.

In other specific embodiments, the cytotoxic or cytostatic agent is auristatin E or a derivative thereof.

In preferred embodiments, the auristatin E derivative is an ester formed between auristatin E and a keto acid. For example, auristatin E can be reacted with paraacetyl benzoic acid or benzoylvaleric acid to produce AEB and AEVB, respectively. Other preferred auristatin derivatives include MMAE and AEFP.

The synthesis and structure of auristatin E, also known in the art as dolastatin-10, and its derivatives are described in U.S. Pat. application Ser. Nos. 09/845,786 and 10/001,191; in the International Patent Application No.: PCT/US02/13435, in U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, all of which are incorporated by reference in their entireties herein.

In specific embodiments, the drug is a DNA minor groove binding agent. Examples of such compounds and their syntheses are disclosed in U.S. Pat. No. 6,130,237, which is incorporated by reference in its entirety herein. In certain embodiments, the drug is a CBI compound.

In certain embodiments of the invention, an ADC of the invention comprises an anti-tubulin agent. Anti-tubulin agents are a well established class of cancer therapy compounds. Examples of anti-tubulin agents include, but are not limited to, taxanes (e.g., Taxol® (paclitaxel), docetaxel), T67 (Tularik), vincas, and auristatins (e.g., auristatin E, AEB, AEVB, MMAE, AEFP). Antitubulin agents included in this class are also: vinca alkaloids, including vincristine and vinblastine, vindesine and vinorelbine; taxanes such as paclitaxel and docetaxel and baccatin derivatives, epithilone A and B, nocodazole, colchicine and colcimid, estramustine, cryptophysins, cemadotin, maytansinoids, combretastatins, dolastatins, discoderrnolide and eleutherobin.

In a specific embodiment, the drug is a maytansinoid, a group of anti-tubulin agents. In a more specific embodiment, the drug is maytansine. Further, in a specific embodiment, the cytotoxic or cytostatic agent is DM-1 (ImmunoGen, Inc.; see also Chari et al., 1992, Cancer Res 52:127-131). Maytansine, a natural product, inhibits tubulin polymerization resulting in a mitotic block and cell death. Thus, the mechanism of action of maytansine appears to be similar to that of vincristine and vinblastine. Maytansine, however, is about 200 to 1,000-fold more cytotoxic in vitro than these vinca alkaloids.

In another specific embodiment, the drug is an AEFP.

In certain specific embodiments of the invention, the drug is not a polypeptide of greater than 50, 100 or 200 amino acids, for example a toxin. In a specific embodiment of the invention, the drug is not ricin.

In other specific embodiments of the invention, an ADC of the invention does not comprise one or more of the cytotoxic or cytostatic agents the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, vinca alkaloids, purine antagonists, and dihydrofolate reductase inhibitors. In more specific embodiments, the high potency drug is not one or more of an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscarnet, and trifluridine.

5.10.1 Dolastatin Drugs

In certain embodiments, the cytotoxic or cytostatic agent is a dolastatin. In more specific embodiments, the dolastatin is of the auristatin class. In a specific embodiment of the invention, the cytotoxic or cytostatic agent is MMAE (MMAE; Formula XI). In another specific embodiment of the invention, the cytotoxic or cytostatic agent is AEFP (Formula XVI).

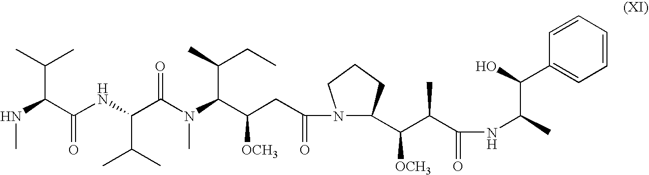

In certain embodiments of the invention, the cytotoxic or cytostatic agent is a dolastatin of formulas XII-XVIII.

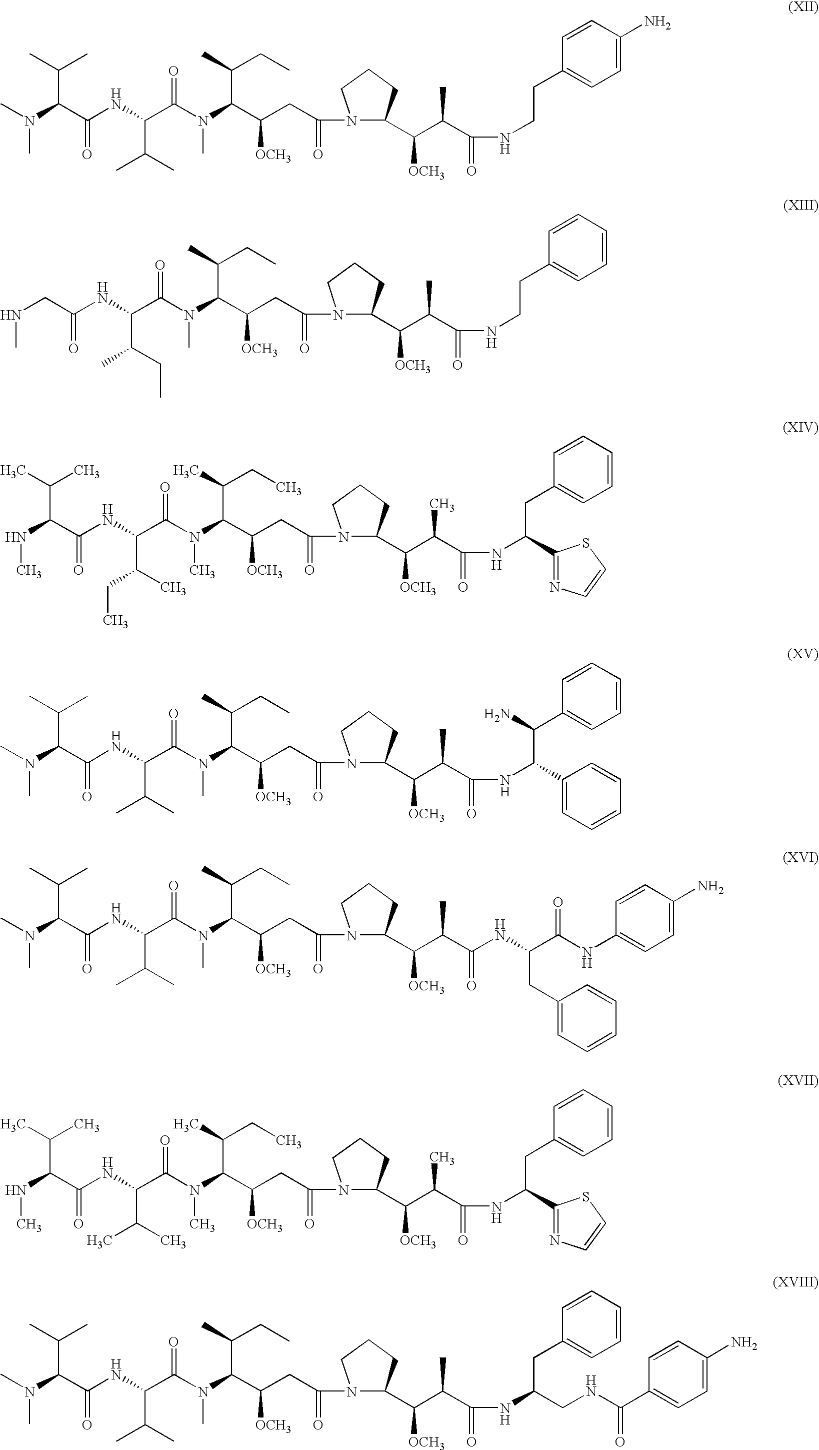

5.10.2 Formation of Anti-CD30 Antibody-Drug Conjugates

The generation of anti-CD30 antibody drug conjugates (ADCs) can be accomplished by any technique known to the skilled artisan. Briefly, the anti-CD30 ADCs comprise an anti-CD30 antibody, a drug, and a linker that joins the drug and the antibody. A number of different reactions are available for covalent attachment of drugs to antibodies. This is often accomplished by reaction of the amino acid residues of the antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of the antibody molecule. Also available for attachment of drugs to antibodies is the Schiff base reaction. This method involves the periodate oxidation of a drug that contains glycol or hydroxy groups, thus forming an aldehyde which is then reacted with the antibody molecule. Attachment occurs via formation of a Schiff base with amino groups of the antibody molecule. Isothiocyanates can also be used as coupling agents for covalently attaching drugs to antibodies. Other techniques known to the skilled artisan and within the scope of the present invention. Non-limiting examples of such techniques are described in, e.g., U.S. Pat. Nos. 5,665,358, 5,643,573, and 5,556,623, which are incorporated by reference in their entireties herein.

In certain embodiments, an intermediate, which is the precursor of the linker, is reacted with the drug under appropriate conditions. In certain embodiments, reactive groups are used on the drug and/or the intermediate. The product of the reaction between the drug and the intermediate, or the derivatized drug, is subsequently reacted with the anti-CD30 antibody under appropriate conditions. Care should be taken to maintain the stability of the antibody under the conditions chosen for the reaction between the derivatized drug and the antibody.

5.11 Gene Therapy

In a specific embodiment, nucleic acids of the invention are administered to treat, inhibit or prevent HD. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; May, 1993, TIBTECH 1, 1(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the therapeutic comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue- specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438. In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid- carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration in linkage to a peptide which is known to enter the nucleus; administration in linkage to a ligand subject to receptor-mediated endocytosis (see, e.g., Wu and Wu, 1987, J. Biol. Chem. 262:4429-4432) (which can be used to target cell types specifically expressing the receptors); etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06 180; WO 92/22635; W092/20316; W093/14188, and WO 93/20221). Alternatively, the nucleic acid can be introduced intracellularly and incorporated within host cell DNA for expression by homologous recombination (Koller and Smithies, 1989, Proc. Natl. Acad. Sci. USA 86:8932-8935; Zijlstra et al., 1989, Nature 342:435-438).

In a specific embodiment, viral vectors that contain nucleic acid sequences encoding an antibody of the invention are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, thereby facilitating delivery of the gene into a patient. More detail about retroviral vectors can be found in Boesen et al., 1994, Biotherapy 6:29 1-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a gene, e.g. an AC10 or HeFi-1 gene, to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to fibroblasts; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of an protein or pharmaceutical composition include determining the effect of the protein or pharmaceutical composition on a Hodgkin's cell line or a tissue sample from a patient with Hodgkin's Disease. The cytotoxic and/or cytostatic effect of the protein or composition on the Hodgkin's cell line and/or tissue sample can be determined utilizing techniques known to those of skill in the art. A preferred method, described in Section 6 infra, entails contacting a culture of the Hodgkin's Disease cell line grown at a density of approximately of about 5,000 cells in a 0.33 cm² of culture area for a period of 72 hours with the protein or pharmaceutical composition, exposing the culture to 0.5 μCi of ³H-thymidine during the final 8 hours of said 72-hour period, and measuring the incorporation of ³H-thymidine into cells of the culture. The protein or pharmaceutical composition has a cytostatic or cytotoxic effect on the Hodgkin's Disease cell line and is useful for the treatment or prevention of Hodgkin's Disease if the cells of the culture have reduced ³H-thymidine incorporation compared to cells of the same Hodgkin's Disease cell line cultured under the same conditions but not contacted with the protein or pharmaceutical composition. Alternatively, in vitro assays which can be used to determine whether administration of a specific protein or pharmaceutical composition is indicated, include in vitro cell culture assays in which a tissue sample from a Hodgkin's Disease patient is grown in culture, and exposed to or otherwise a protein or pharmaceutical composition, and the effect of such compound upon the Hodgkin's tissue sample is observed.

5.12 Therapeutic/Prophylactic Administration and Compositions

The invention provides methods of treatment and prophylaxis by administration to a subject of an effective amount of a CD30-binding protein which has a cytotoxic or cytostatic effect on Hodgkin's Disease cells (i.e., a protein of the invention), a nucleic acid encoding said CD30-binding protein (i.e., a nucleic acid of the invention), or a pharmaceutical composition comprising a protein or nucleic acid of the invention (hereinafter, a pharmaceutical of the invention). According to the present invention, treatment of HD encompasses the treatment of patients already diagnosed as HD at any clinical stage; such treatment resulting in delaying tumor growth; and/or promoting tumor regression.

In a preferred embodiment, the protein of the invention is the monoclonal antibody AC10 or HeFi-1 or a fragment or derivative thereof. In a preferred aspect, a pharmaceutical of the invention comprises a substantially purified protein or nucleic acid of the invention (e.g., substantially free from substances that limit its effect or produce undesired side-effects). In various embodiments, the protein or nucleic acid is at least 50%, 60%, 70%, 80% or 90% pure.

The subject is preferably an animal, including but not limited to animals such as cows, pigs, horses, chickens, cats, dogs, etc., and is preferably a mammal, and most preferably human.

Formulations and methods of administration that can be employed are described above; additional appropriate formulations and routes of administration can be selected from among those described herein below.

Various delivery systems are known and can be used to administer a nucleic acid or protein of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction include but are not limited to intradermal, intramuscular, intraperitoneal, intravenous, subcutaneous, intranasal, epidural, and oral routes. Nucleic acids and proteins of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents such as chemotherapeutic agents (see Section). Administration can be systemic or local.

In a specific embodiment, it may be desirable to administer the nucleic acid or protein of the invention by injection, by means of a catheter, by means of a suppository, or by means of an implant, said implant being of a porous, non-porous, or gelatinous material, including a membrane, such as a sialastic membrane, or a fiber. Preferably, when administering a protein, including an antibody, of the invention, care must be taken to use materials to which the protein does not absorb.

In another embodiment, the compound or composition can be delivered in a vesicle, in particular a liposome (see Langer, 1990, Science 249:1527-1533; Treat et al., 1989, in Liposomes in the Therapy of Infectious Disease and Cancer, Lopez-Berestein and Fidler (eds.), Liss, New York, pp. 353-365; Lopez-Berestein, ibid., pp. 317-327; see generally, ibid.)

In yet another embodiment, the compound or composition can be delivered in a controlled release system. In one embodiment, a pump may be used (see Langer, supra; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

Other controlled release systems are discussed in the review by Langer, 1990, Science 249:1527-1533.

In a specific embodiment where a nucleic acid of the invention is administered, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox- like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

As alluded to above, the present invention also provides pharmaceutical compositions (pharmaceuticals of the invention). Such compositions comprise a therapeutically effective amount of a nucleic acid or protein of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the nucleic acid or protein of the invention, preferably in purified form, together with a suitable amount of carrier so as to provide the form for proper administration to the patient. The formulation should suit the mode of administration.

In a preferred embodiment, the pharmaceutical of the invention is formulated in accordance with routine procedures as a pharmaceutical composition adapted for intravenous administration to human beings. Typically, compositions for intravenous administration are solutions in sterile isotonic aqueous buffer. Where necessary, the pharmaceutical of the invention may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical of the invention is to be administered by infusion, it can be dispensed with an infusion bottle containing sterile pharmaceutical grade water or saline. Where the pharmaceutical of the invention is administered by injection, an ampoule of sterile water for injection or saline can be provided so that the ingredients may be mixed prior to administration.

The amount of the nucleic acid or protein of the invention which will be effective in the treatment or prevention of HD can be determined by standard clinical techniques. In addition, in vitro assays may optionally be employed to help identify optimal dosage ranges. The precise dose to be employed in the formulation will also depend on the route of administration, and the stage of HD, and should be decided according to the judgment of the practitioner and each patient's circumstances. Effective doses may be extrapolated from dose-response curves derived from in vitro or animal model test systems.

5.13 Kits

The invention also provides a pharmaceutical pack or kit comprising one or more containers filled with a nucleic acid or protein of the invention and optionally one or more pharmaceutical carriers. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, a kit comprises a purified protein of the invention. In a preferred mode of the embodiment, the protein is an antibody. The protein may be conjugated to a radionuclide or chemotherapeutic agent. The kit optionally further comprises a pharmaceutical carrier.

In another embodiment, a kit of the invention comprises a nucleic acid of the invention, or a host cell comprising a nucleic acid of the invention, operably linked to a promoter for recombinant expression.

5.14 Effective Dose

Toxicity and therapeutic efficacy of the proteins of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Proteins that exhibit large therapeutic indices are preferred. While proteins that exhibit toxic side effects may be used, care should be taken to design a delivery system that targets such proteins to the site of affected tissue in order to minimize potential damage to uninfected cells and, thereby, reduce side effects.

The data obtained from the cell culture assays and animal studies can be used in formulating a range of dosage for use in humans. The dosage of such proteins lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Generally, the dosage of a protein of the invention in a pharmaceutical of the invention administered to a Hodgkin's Disease patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign proteins. Thus, lower dosages of humanized, chimeric or human antibodies and less frequent administration is often possible.

5.15 Formulations

Pharmaceutical compositions for use in accordance with the present invention may be formulated in conventional manner using one or more physiologically acceptable carriers or excipients.

Thus, the proteins and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate) lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the proteins for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The proteins may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The proteins may also be formulated in rectal compositions such as suppositories or retention enemas, e.g., containing conventional suppository bases such as cocoa butter or other glycerides.

In addition to the formulations described previously, the proteins may also be formulated as a depot preparation. Such long acting formulations may be administered by implantation (for example subcutaneously or intramuscularly) or by intramuscular injection. Thus, for example, the proteins may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration preferably for administration to a human.

5.16 Combination Therapy for Treatment of Hodgkin's Disease

The nucleic acids and proteins of the invention can be administered together with treatment with irradiation or one or more chemotherapeutic agents. In specific embodiments, the chemotherapeutic agent is a cytostatic, cytotoxic, and/or immunosuppressive agent.

In certain specific embodiments, the immunosuppressive agent is gancyclovir, acyclovir, etanercept, rapamycin, cyclosporine or tacrolimus. In other embodiments, the immunosuppressive agent is an antimetabolite, a purine antagonist (e.g., azathioprine or mycophenolate mofetil), a dihydrofolate reductase inhibitor (e.g., methotrexate), a glucocorticoid. (e.g., cortisol or aldosterone), or a glucocorticoid analogue (e.g., prednisone or dexamethasone). In yet other embodiments, the immunosuppressive agent is an alkylating agent (e.g., cyclophosphamide). In yet other embodiments, the immunosuppressive agent is an anti-inflammatory agent, including but not limited to a cyclooxygenase inhibitor, a 5-lipoxygenase inhibitor, and a leukotriene receptor antagonist.

For irradiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds., 2nd. Ed., J. B. Lippencott Company, Philadelphia.

Useful classes of chemotherapeutic agents include, but are not limited to, the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids. Individual chemotherapeutics encompassed by the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In a specific embodiment, a nucleic acid or protein of the invention is administered concurrently with radiation therapy or one or more chemotherapeutic agents. In another specific embodiment, chemotherapy or radiation therapy is administered prior or subsequent to administration of a nucleic acid or protein of the invention, by at least an hour and up to several months, for example at least an hour, five hours, 12 hours, a day, a week, a month, or three months, prior or subsequent to administration of a nucleic acid or protein of the invention.

In a specific embodiment in which a protein of the invention is conjugated to a pro-drug converting enzyme, or in which a nucleic acid of the invention encodes a fusion protein comprising a pro-drug converting enzyme, the protein or nucleic acid is administered with a pro-drug. Administration of the pro-drug can be concurrent with administration of the nucleic acid or protein of the invention, or, more preferably, follows the administration of the nucleic acid or protein of the invention by at least an hour to up to one week, for example about five hours, 12 hours, or a day. Depending on the pro-drug converting enzyme administered, the pro-drug can be a benzoic acid mustard, an aniline mustard, a phenol mustard, p-hydroxyaniline mustard-glucuronide, epirubicin-glucuronide, adriamycin-N phenoxyaceryl, N-(4′-hydroxyphenyl acetyl)-palytoxin doxorubicin, melphalan, nitrogen mustard-cephalosporin, β-phenylenediamine, vinblastine derivative-cephalosporin, cephalosporin mustard, cyanophenylmethyl-β-D-gluco-pyranosiduronic acid, 5-(adaridin-1-yl-)2, 4-dinitrobenzamide, or methotrexate-alanine.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The invention is further described in the following examples which are in no way intended to limit the scope of the invention.

### 6. EXAMPLE

### Anti-CD30 Monoclonal Antibodies AC10 and HEFI-1 Inhibit the Growth of CD30-Expressing Hodgkin's Disease Cell Lines

6.1 Materials and Methods

Cells and culture conditions: The CD30 expressing cell lines, L540, HDLM2, L428, KM-H2 and Karpas 299. were obtained from the German Collection of Microorganisms and Cell Cultures/DSMZ in Braunschweig, Germany. The Hodgkin's cell line L540cy was a provided by Dr. V. Diehl of the University of Cologne, Cologne, Germany. The cell lines were maintained in the recommended media formulations and subcultured every 3-4 days.

Reagents and antibodies: Anti-CD30 monoclonal antibody hybridoma line AC10 was described by Bowen et al. (Bowen et al., 1993, J. Immunol. 151:5896-5906) and was provided by Dr. E. Podack, University of Miami. Purified antibody was isolated from serum-free supernatants using a protein-G immunoaffinity column. The resulting AC10 antibody was determined to be >97% monomeric by size exclusion chromatography. The monoclonal antibody HeFi-1 has been previously described and was provided by Dr. T. Hecht, NCI, Bethesda, M D. HeFi-1 mAb was demonstrated by size exclusion chromatography to be greater than 98% monomer.

Proliferation assays: CD30 expressing cell lines were cultured in flat-bottom 96-well plates at a density of 50,000 or 5,000 cells/well in growth media (RPMI with 10% (heat-inactived) fetal bovine serum (FBS) for cell lines L428, KM-H2 and Karpas 299, and RPMI/20% (heat inactivated) FBS for cell lines HDLM-2 and L540. The cell lines were cultured in the absence or presence of cross-linked soluble anti-CD30 mAbs or immobilized anti-CD30 mAbs, as described below.

Antibody cross-linking in solution: To cross-link the anti-CD30 antibodies in solution, various dilutions of AC10 or HeFi-1 were titrated into 96-well flat bottom tissue culture plates in the absence or presence of 20 μg/ml polyclonal goat anti-mouse IgG antibodies. Hodgkin's disease cell lines were then added to the plates at either 50,000 or 5,000 cells/well. The plates were incubated at 37° C. for 72 hours and were labeled with ³H-thymidine, 1 μCi/well, for the final 5 hours.

Antibody immobilization: Antibody immobilization was obtained by coating wells with antibody in 50 mmol/L Tris buffer (pH 8.5) for 18 hours at 4° C. Prior to the addition of cells, wells were washed twice with PBS to remove unbound mAb. 50,000 or 5,000 cells in a total volume of 200 μl were added to each well. Proliferation was determined by uptake of ³H-thymidine (0.5 μCi/well) during the final 8 hours of a 72 hour culture period.

6.2 Results

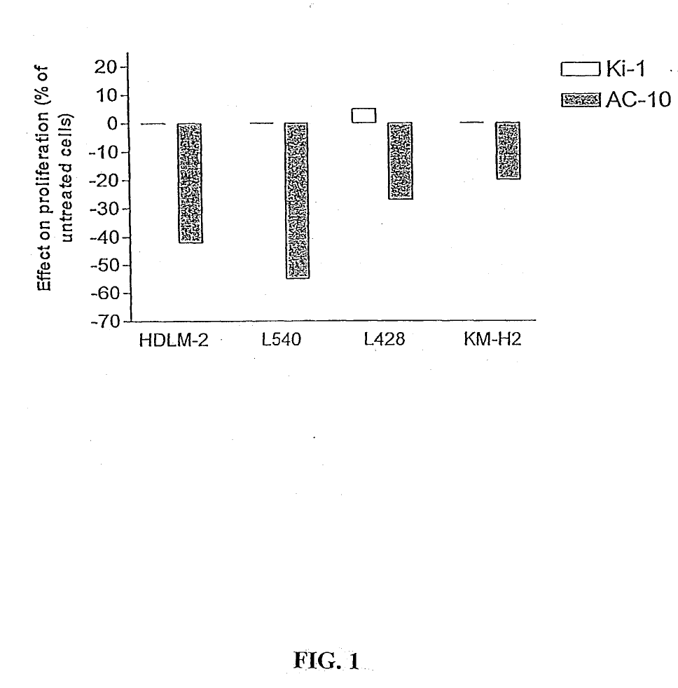

To evaluate the biologic activity of anti-CD30 mAbs, CD30-expressing HD cell lines (50,000 cells/well) were cultured in the presence of immobilized anti-CD30 mAb AC10. mAb AC10 demonstrated inhibition of cell growth of T-cell-like (L540 and HDLM-2) or B-cell-like (L428 and KM-H2) HD lines (FIG. 1). Ki-1, which was previously shown to have no effect on HD cell lines (Gruss et al., 1996, Blood 83:2045-2056), was used as a control.

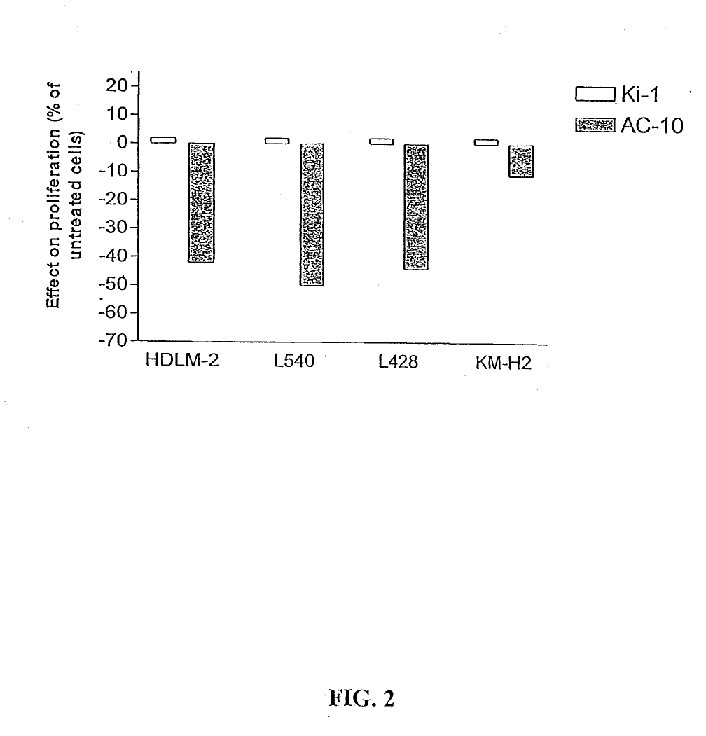

To further evaluate the activity of AC10, a second series of assays were performed. In order to assess the activity of the AC10 during a period of logarithmic tumor cell growth, the cell density of the cultures was decreased to provide more optimal growth conditions. To that end, HD cell lines were cultured in flat-bottom 96 well plates at a density of 5,000 cells/well in the presence or absence of mAb AC10. AC10 demonstrated growth inhibition of all four HD cell lines tested (L540, HDLM-2, L428 and KM-H2; FIG. 2).

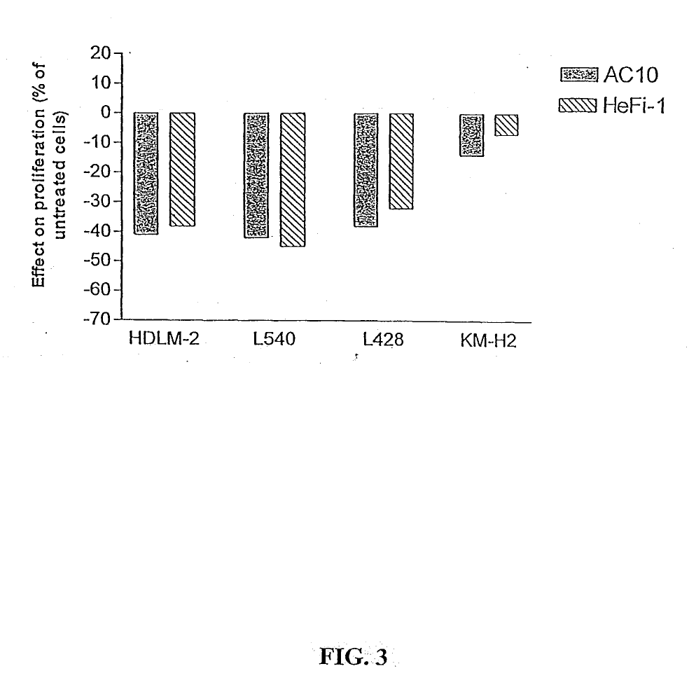

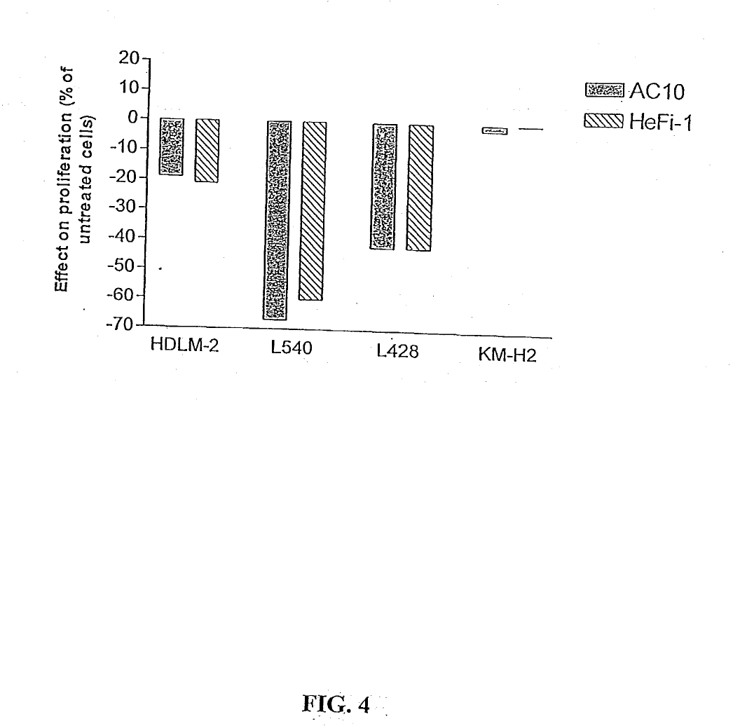

In another set of experiments, HD cell lines were incubated with soluble AC10 or HeFi-1 that were cross-linked in solution by the addition of soluble goat anti-mouse IgG antibodies. Under these cross-linking conditions, all four HD cell lines, when plated at 5×10⁴ cell/well, were growth inhibited by AC10 and HeFi-1 (FIG. 3). When the cells were plated at 5×10³ cell/well, AC10 inhibited the growth of HDLM-2, L540, and L428 and, to a lesser extent, the cell line KM-H2, while HeFi-1 inhibited the growth of the cell lines HDLM-2, L540, and L428 (FIG. 4).

The data resulting from the experiments testing the effects of AC10 and HeFi-1 on CD30-expressing tumor cell lines are summarized in Table 2, infra. Table 2 further provides a comparison of the anti-tumor activity of AC10 and HeFi-1 with that of mAb M44.

TABLE 2
Cytostatic and/or cytotoxic activity of signaling
anti-CD30 mAbs on
CD30-expressing malignant cell lines

| | | Inhibition of Growth by | | |
|---|---|---|---|---|
| Cell Line | Cell Type | M44^{a} | HeFi-1 | AC10 |
| Karpas 299 | ALCL | + | + | + |
| Michel | ALCL | + | ND | ND |
| KM-H2 | HD (B cell phenotype) | − | + | + |
| L428 | HD (B cell phenotype) | − | + | + |
| HDLM-2 | HD (T cell phenotype) | − | + | + |
| L540 | HD (T cell phenotype) | − | + | + |

^{a}Published data from Gruss et al, Blood 83(8): 2045-2056

Taken together, these data indicate that mAbs AC10 and HeFi-1 are distinguished from the previously described anti-CD30 mAbs by their ability to inhibit the growth of CD30-expressing HD lines. It is of interest to note that Hubinger et al. recently evaluated the activity of the anti-CD30 mAb M44, in immobilized form, in a proliferation assay utilizing 5,000 cells/well. Under these conditions, M44 inhibited the growth of the CD30-expressing ALCL line, Karpas 299 but not the HD cell line HDLM-2 (Hubinger et al., 1999, Exp. Hematol. 27(12):1796-805).

### 7. AC10 Enhances the Cytotoxic Effect of Chemotherapeutics on Hodgkin's Disease Cell Lines

7.1 Materials and Methods

L428 cells were cultured for 24 hours in the presence or absence of 0.1 μg/ml anti-CD30 antibody, AC10, crosslinked by the addition of 20 μg/ml goat anti-mouse IgG antibodies. After the 24-hour culture period, the cells were harvested and washed with phosphate buffered saline (PBS). The cells were then plated into 96-well flat-bottom tissue culture plates at 5×10³ cells/well and mixed with various dilutions of chemotherapeutic drugs. After a 1-hour exposure to the drugs the cells were washed twice, followed by the addition of fresh culture media. The plates were then incubated at 37° C. for 72 hours followed by a 4-hour incubation with 0.5 μCi/well ³H-thymidine. The inhibition of growth was determined by comparing the amount of ³H-thymidine incorporated into treated cells to the amount incorporated into untreated control cells.

7.2 Results

To evaluate the effect of the anti-CD30 mAb in combination with chemotherapeutic drugs, L428 cells were incubated for 24 hours in either the absence of antibody or the presence of AC10 at 0.1 μg/ml with 20 μg/ml goat anti-mouse IgG to provide crosslinking for the primary antibody. After this incubation the cells were plated into 96-well tissue culture plates at 5×10³ cells/well in the presence of dilutions of chemotherapeutic drugs including doxorubicin, cisplatin, and etoposide (Table 3). The EC₅₀, concentration of drug needed to inhibit the incorporation of ³H-thymidine by 50% compared to untreated control cells, was then determined for cells treated with the drugs alone or the combinations of drug and antibody. For doxorubicin, incubation with AC10 decreased the EC₅₀ on L428 cells (i.e. decreased the amount of drug necessary to inhibit 50% of DNA synthesis) from approximately 45 nM (doxorubicin alone) to approximately 9 nM, for cisplatin AC10 decreased the EC₅₀ from ˜1,500 nM to ˜500 nM, and for etoposide AC10 decreased the EC₅₀ from ˜1,500 nM to ˜600 nM.

TABLE 3
AC10 enhances the effectiveness of chemotherapeutic
drugs on the HD cell line L428.

| | EC₅₀, nM | |
|---|---|---|
| Drug | without AC10 | with AC10 |
| Doxorubicin | 45 | 9 |
| Cisplatin | 1,500 | 500 |
| Etoposide | 1,500 | 600 |

### 8. Antitumor Activity of AC10 and HEFI-1 in Disseminated and Localized (Subcutaneous) L540CY Hodgkin's Disease Xenografts

8.1 Materials and Methods

Human tumor xenograft models: Female C.B-17 SCID mice, obtained from Taconic (Germantown, N.Y.) at 4-6 weeks of age, were used for all efficacy studies. To establish xenograft models of Hodgkin's disease, L540cy (HD) cells were harvested from cell culture, washed in ice cold phosphate buffered saline (PBS), resuspended in PBS, and maintained on ice until implantation. For disseminated disease models, mice were injected intravenously through the tail vein with 10⁷ L540cy cells. Solid tumor xenografts were established by injecting mice subcutaneously (s.c.) with 2×10⁷ L540cy cells. For therapeutic evaluation the indicated treatment doses and schedules were used.

Administration of AC10 and HeFi-1: Disseminated L540cy tumor bearing mice received 10⁷ cells through the tail vein on d0 followed by therapy initiated on d1. Treated mice received i.p. injections of either AC10 or HeFi-1 every two days for a total of 10 injections, q2d×10, at 1 mg/kg/injection.

For the subcutaneous L540cy model, mice were injected s.c. with 2×10⁷ cells and were observed daily for solid tumor formation. When tumors were palpable, the animals were randomly distributed into groups and received either AC10 or HeFi-1 q2d×10 at 2 mg/kg/injection.

8.2 Results

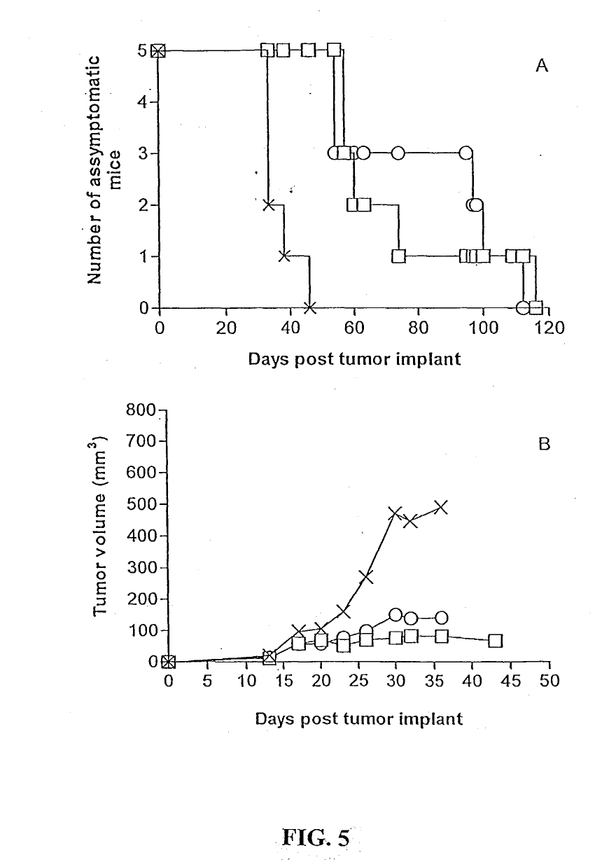

AC10 and HeFi-1 were tested in L540cy Hodgkin's disease xenografted SCID mice, as described above. In the mouse population with disseminated L540cy tumors, all of the untreated control animals developed signs of severe disseminated disease such as hind limb paralysis or the formation of a solid tumor mass and had to be sacrificed (mean survival time=37 days). In contrast, all of the mice that received either AC10 or HeFi-1 survived for >46 days with no signs of disease (FIG. 5A).

With respect to the mouse population with subcutaneous L540cy tumors, while the untreated control tumors rapidly grew to >450 mm³, both mAbs significantly delayed tumor growth as shown in FIG. 5B.

The inventors have identified murine monoclonal antibodies (mAbs) which target the human CD30 receptor and display a profile of activity not previously described for other anti-CD30 mAbs. In unmodified form, these antibodies, AC10 and HeFi-1 inhibit the growth of HD and the ALCL line Karpas 299 and display in vivo antitumor activity in a tumor xenograft model of Hodgkin's disease.

### 9. In Vitro Activities of Chimeric AC10

9.1 Materials and Methods

Cells and reagents: The AC10 hybridoma was grown in RPMI-1640 media (Life Technologies Inc. Gaithersburg, Md.) supplemented with 10% fetal bovine serum. Antibody was purified from culture supematants by protein A chromatography. CD30-positive HD lines L540, KM-H2, HDLM-2 and L428, as well as the ALCL line Karpas-299, were obtained from the Deutsche Sammlung von Mikroorganismen und Zellkulturen GmbH (Braunschweig, Germany); L540cy was provided by Dr. V. Diehl; HL-60 and Daudi were obtained from ATCC (Manassas, Va.). DG44 CHO cells were obtained from Lawrence Chasm (Columbia University, New York, N.Y.). Goat-anti-mouse-FITC or goat-anti-human-FITC were from Jackson Immunoresearch, (West Grove, Pa.). Anti-CD30 mAb Ki-1 was from Accurate Chemicals (Westbury, N.Y.).

FACS analysis: To evaluate CD30 expression on cell lines, 3×10⁵ cells were combined with saturating levels (4 μg/ml) of either AC10 or chimeric AC10 (cAC10) in ice-cold 2% FBS/PBS (staining media) for 20 mm on ice and washed twice with ice-cold staining media to remove unbound mAb. Cells were then stained with secondary mAbs diluted 1:50 in ice-cold staining media, goat-anti-mouse FITC for AC10 or goat-anti-human-FITC for cAC10, incubated for 20 minutes on ice, washed as described above and resuspended in 5 μg/mL propidium iodide (PI). Labeled cells were examined by flow cytometry on a Becton Dickinson FACScan flow cytometer and were gated to exclude the non-viable cells. Data was analyzed using Becton Dickinson CellQuest software version 3.3 and the background-corrected mean fluorescence intensity was determined for each cell type.

For antibody saturation binding, 3×10⁵ Karpas 299 cells were combined with increasing concentrations of AC10 or cAC10 diluted in ice-cold staining media for 20 minutes on ice, washed twice with ice-cold staining media to remove free mAb and incubated with 1:50 goat-anti-mouse-FITC or goat-anti-human-FITC. respectively. The labeled cells were washed, resuspended in P1 and analyzed as described above. The resultant mean fluorescence intensities were plotted versus mAb concentration.

For analysis of cell cycle position cells were cultured in complete media and at the indicated times were labeled with bromodeoxynridine (BrdU) (10 μM final; Sigma, St.Louis, Mo.) for 20 mm to detect nascent DNA synthesis, and with PI to detect total DNA content as previously described (Donaldson et al., 1997, J. Immunol. Meth. 203:25-33). Labeled cells were analyzed for cell cycle position and apoptosis by flow cytometry using the Becton-Dickinson CellQuest program as previously described (Donaldson et al., 1997, J. Immunol. Meth. 203:25-33).

In vitro growth inhibition: Evaluation of growth inhibition by murine mAbs was carried out by immobilizing the mAb at 10 μg/mL in 50 mM Tris-HCl pH 8.5, to plastic 96-well tissue culture plates overnight at 4° C. Plates were washed twice with PBS to remove unbound mAb followed by addition of cells in 100 μl complete media at 5,000 cells/well. Following 48 h incubation at 37° C., 5% CO₂, cells were labeled with ³H-TdR by the addition of 50 μl complete media containing 0.5 μCi of ³H-TdR for 2 h and the level of DNA synthesis determined relative to cells in untreated control wells. Evaluation of growth inhibition by cAC10 was carried out using soluble mAb and secondary crosslinker. Cells were plated at 5,000 cells/well in 180 μl complete media in a 96-well format. cAC10 in complete media containing a corresponding 10-fold excess of goat-anti-human IgG was added at the concentrations noted, in 20 μl. At 96 h postincubation cells were labeled with ³H-TdR for 4 h followed by cell harvest and scintillation counting to quantify the level of nascent DNA synthesis. The percent inhibition relative to untreated control wells was plotted versus cAC10 concentration.

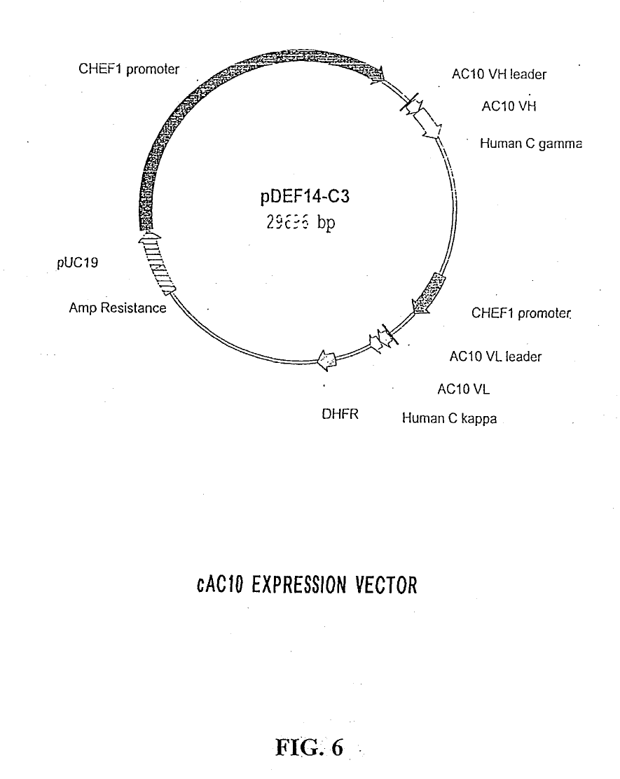

Construction and expression of chimeric AC10 (cAC10): For construction of cAC10, the heavy chain and light chain variable regions were cloned from the AC10 hybridoma using the methods of Gilliland et al., 1996, Tissue Antigens 47:1-20. Total RNA was isolated from the AC10 hybridoma and cDNA of the variable regions was generated using mouse kappa and IgG2b gene-specific primers. DNA encoding the AC10 heavy chain variable region (VH) was joined to sequence encoding the human gamma I constant region (huCγl, SwissProt accession number P01857) in a cloning vector and the AC10 light chain variable region (VL) was similarly joined to the human kappa constant region (huCκ, PID G185945) in a separate cloning vector. Both the heavy and light chain chimeric sequences were cloned into pDEF14 for expression of intact chimeric monoclonal antibody in CHO cells. The plasmid pDEF14 utilizes the Chinese hamster elongation factor 1 alpha gene promoter that drives transcription of heterologous genes (U.S. Pat. No. 5,888,809) leading to high levels of expression of recombinant proteins without the need for gene amplification. The resulting plasmid was designated pDEF14-C3 (FIG. 6).

For generation of the cAC10 expressing cell line, pDEF14-C3 was linearized and transfected into DG44 CHO cells by electroporation. After electroporation, the cells were allowed to recover for two days in complete DMEM/F12 media containing 10% FBS, after which the media was replaced with selective media without hypoxanthine and thymidine. Only those cells that incorporated the plasmid DNA, which includes the DLIFR gene, were able to grow in the absence of hypoxanthine and thymidine. High titer clones were selected and cultured in bioreactors. cAC10 antibody was purified by protein A, ion exchange, and hydrophobic interaction chromatographies, with the final product determined by HPLC-SEC to be >99% monomer antibody.

9.2 Results

Binding of murine AC10 and chimeric AC10 to Hodgkin 's disease cell lines: AC10 was originally produced by immunizing mice with the CD30-positive large granular lymphoma cell line YT and was shown to be specific for CD30 (Bowen et al., 1993, J. Immunol. 151:5896-5906). Prior to evaluating the effects of AC10 and cAC10 on the growth of HD cells, the levels of CD30 expression on several cultured cell lines were compared. All four HD lines tested were CD30-positive based on flow cytometry fluorescence ratios (Table 4). The T cell-like HD cell lines HDLM-2 and L540 as well as the ALCL line Karpas-299 expressed qualitatively similar, high levels of CD30 while expression on two B cell-like HD lines KM-H2 and L428 were somewhat lower. L540cy, a subclone of L540, displayed an intermediate level of CD30 expression. Although the binding of cAC10 and AC10 to these cell lines was detected using different secondary antibodies—FITC conjugated goat anti-human or goat anti-mouse, respectively—these data demonstrate that the chimerization process did not diminish cAC10-specific binding to cell surface CD30. The promyelocytic leukemia line HL-60 and the Burkitt's lymphoma line Daudi were both CD30-negative and served as controls in subsequent studies.

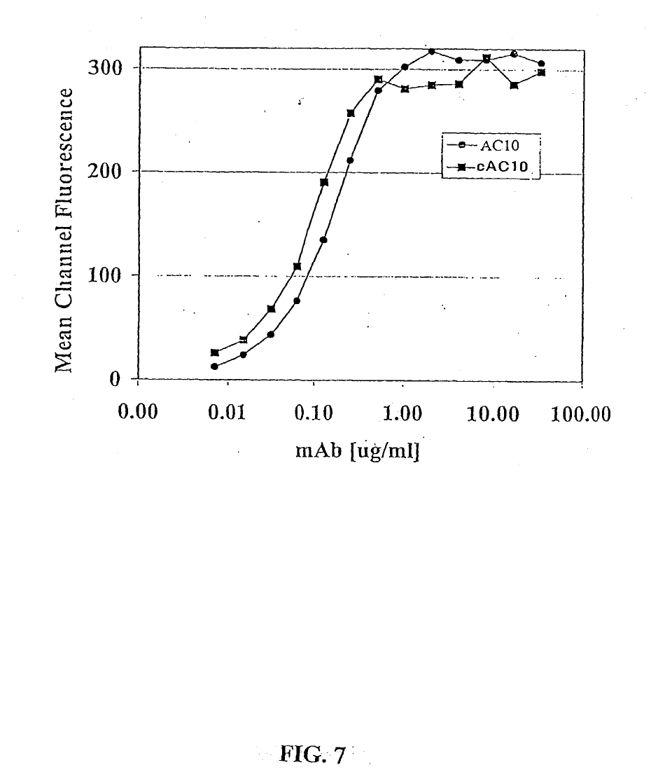

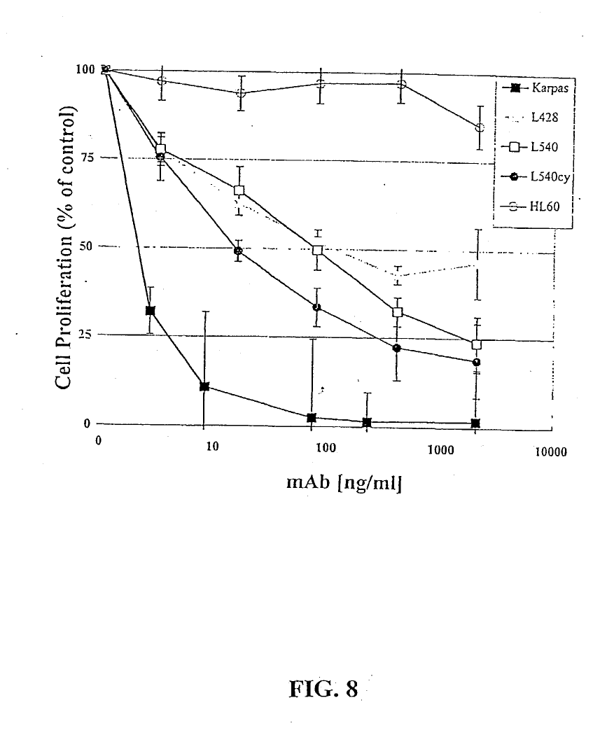

To further compare the binding activity of the murine and chimeric antibodies, Karpas-299 cells were incubated with titrations of AC10 or cAC10 followed by labeling with goat-anti-mouse-FITC or goat-anti-human-FITC (Jackson Immunoresearch, West Grove, Pa.), respectively, to determine levels required for saturation. Labeled cells were examined by flow cytometry and the mean fluorescence intensity plotted against mAb concentration. Binding saturation for both forms of the mAb occurred at ˜0.5 μg/ml (FIG. 7). Saturation was consistent for all CD30-positive cell lines examined (data not shown), further demonstrating that cAC10 retained the binding activity of the parental murine antibody.

Freshly isolated peripheral blood mononuclear cells did not react with cAC10 and showed no signal above background in this assay. Similarly, isolated human primary B-cells and T-cells did not bind cAC10. Primary human peripheral T-cells activated with anti-CD3 and anti-CD28, and B-cells activated by pokeweed mitogen both showed transient, low level binding of cAC10 at 72 h-post activation, which diminished thereafter (data not shown).

In vitro activities of AC10 and cAC10: Anti-CD30 antibodies such as M44 and M67 have been shown to have anti-proliferative effects on ALCL lines, while having either no effect or stimulating the growth of HD lines (Gruss et al, 1994, Blood 83:2045-2056; Tian et al., 1995, Cancer Res. 55:5335-5341). To initially evaluate the effect of the mAb AC10 on HD cell proliferation, AC10 was compared to mAb Ki-1 under previously reported solid phase conditions (Gruss et al, 1994, Blood 83:2045-2056). For these studies mAbs were immobilized onto plastic tissue culture plates prior to the addition of HD cells as described in Materials and Methods. Following incubation for 48 h at 37° C., cells were labeled with ³H-TdR and the level of DNA synthesis determined relative to cells in untreated control wells. FIG. 3A shows that the presence of immobilized mAb Ki-1 had nominal effect on the growth of the HD lines. In contrast, the presence of immobilized AC10 resulted in significant growth inhibition.

Following chimerization, a titration of cAC10 was performed on the HD cell lines L540, L540cy and L428 as well as the ALCL line Karpas-299. cAC10 was added in solution at the concentrations noted in the presence of 10-fold excess of goat-anti-human IgG. Cross-linking antibody was added to potentiate the effects of cAC10 and to approximate the effects of FcR-mediated crosslinking that could occur in vivo. The CD30-positive ALCL line was highly sensitive to cAC10, with anIC₅₀ (concentration of mAb that inhibited 50% of cell growth) of 2 ng/ml. The HD lines L428, L540 and L540cy showed IC₅₀ sensitivities to cAC10 of 100 ng/ml, 80 ng/ml and 15 ng/ml respectively. In parallel studies these cells treated with a non-binding control mAb and cross-linker showed no decrease in DNA synthesis over the concentration range tested (data not shown) and the CD30-negative line HL-60 showed only slight inhibition by cAC10 at the highest level tested (FIG. 8).

Cell cycle effects of cAC10: Hubinger et al. have recently shown that anti-CD30 mAbs can inhibit the growth of ALCL cells, including Karpas-299, through induction of cell cycle arrest and without induction of apoptosis (Hubinger et al., 2001, Oncogene 20:590-598). However, these antibodies did not have inhibitory effect on HD cells, and in some cases they stimulated proliferation. To more closely examine the cell cycle effects of cAC10 in vitro, the HD cell line L540cy was cultured in complete media containing 1.0 μg/ml of cAC10 complexed with goat-anti-human IgG at 10 μg/ml. At the indicated times, cells were labeled with bromodeoxyunridine for 20 min to detect nascent DNA synthesis, and with propidium iodine to detect total DNA content. Labeled cells were analyzed for cell cycle position by flow cytometry using the Becton-Dickinson Cellfit program as previously described (Donaldson et al., 1997, J. Immunol. Meth. 203:25-33).

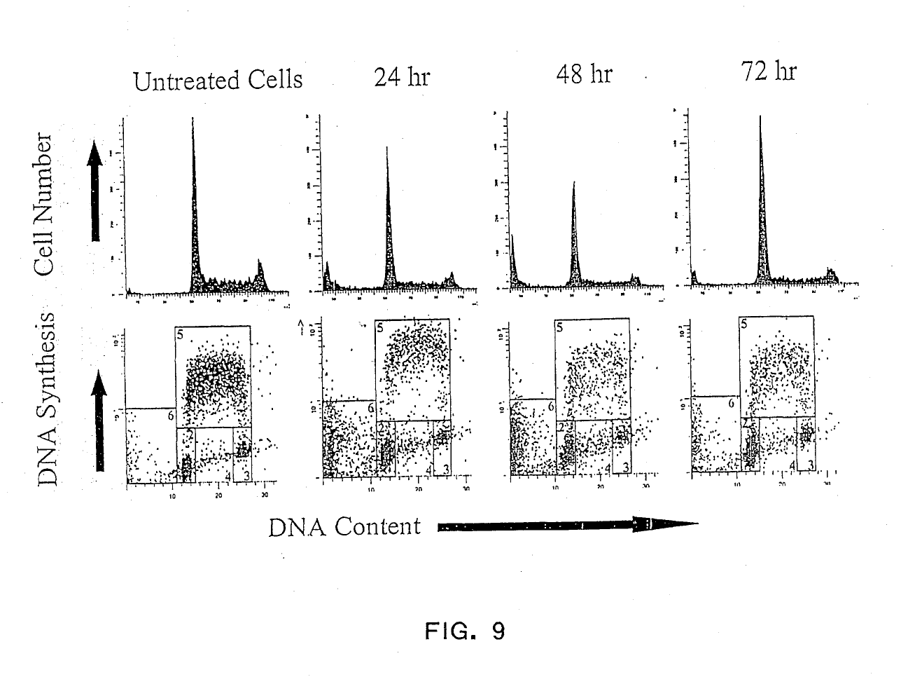

FIG. 9 shows a representative shift in DNA content and DNA synthesis in of L540cy HD cells following exposure to cAC10. The percent of the population in each region was quantified as described in section 9.1 and shown in Table 5. Exposure of L540cy to cAC10 results in time-dependent loss of the S-phase cells from 40% in the untreated population to 13% at 2 days -post exposure. Coordinately, the G₁ content of this population increased from 40% in untreated cells to 65% at 3 days -post exposure. The region of less than G₁ content gives an accurate indication of apoptotic cells undergoing DNA fragmentation (Donaldson et al., 1997, J. Immnunol. Meth. 203:25-33) and this population increased from 6% in the untreated population to 29% at 48 h post cAC10 exposure. These flow cytometric studies were corroborated by a parallel dye exclusion assay using a hemocytometer. As measured by dye exclusion, untreated L540cy cells were 93% viable and this decreased to 72% at 48 h post cAC10 exposure. Karpas cells treated with cAC10 showed a similar decrease in S-phase from 40% to 11% at 48 h post-cAC10 (Table 5). In control studies, the CD30-negative B-cell line Daudi showed only nominal modulation of cell cycle and no increase in apoptosis following treatment with cAC10 (Table 5). Unlike previous studies in which immobilized mAb to CD30 induced apoptosis in ALCL cells (Mir et al, 2000, Blood 96:4307-43 12.), little to no apoptosis on these cells with soluble cAC10 and a crosslinking secondary antibody was observed. Taken together, these data demonstrate cAC10 induced growth arrest and accumulation of the G₁ population and diminution of S-phase in both CD30-positive lines, and induction of apoptosis in L540cy HD cells in vitro.

TABLE 4
Binding of AC10 and cAC10 to different cell lines.

| MFIb | | | | Binding Ratiosc | |
|---|---|---|---|---|---|
| Cell Line | Lineage^{a} | AC10 | cAC10 | AC10 | cAC10 |
| HDLM2 | Hodgkin's Disease | 507.2 | 591.8 | 156 | 176 |
| | (T-cell like) | | | | |
| L540 | Hodgkin's Disease | 435.8 | 582.5 | 183 | 251 |
| | (T-cell like) | | | | |
| L540cy | Hodgkin's Disease | 363.3 | 495.9 | 120 | 156 |
| | (T-cell like) | | | | |
| Karpas | Anaplastic Large Cell | 399.9 | 579.2 | 158 | 176 |
| | Lymphoma | | | | |
| KM-H2 | Hodgkin's Disease | 102.0 | 105.8 | 33 | 41 |
| | (B-cell like) | | | | |
| L428 | Hodgkin's Disease | 174.4 | 186.0 | 67 | 67 |
| | (B-cell like) | | | | |
| HL60 | Acute Myelogenous | 1.0 | 3.8 | 1 | 2 |
| | Leukemia | | | | |
| Daudi | Burkitt's Lymphoma | −0.6 | 0.9 | 1 | 1 |
| | B-cell | | | | |

^{a}Gruss et al., 1994
^{b}Mean Fluorescence Intensity
^{c}Binding ratios were determined by dividing the geometric mean fluorescence intensity of cells stained with primary (AC10 or cAC10 at 4 μg/ml) and appropriate secondary (goat anti-mouse or goat anti-human Ig respectively) - FITC conjugate, by the geometric mean fluorescence intensity of cells stained with respective secondary antibody alone.

TABLE 5
Cell cycle effects of cAC10.

| | | | |
|---|---|---|---|
| Untreated | 24 hr | 48 hr | 72 hr |

L540cy

| | | | | |
|---|---|---|---|---|
| % G1 | 40 | 52 | 51 | 65 |
| % S | 40 | 21 | 13 | 17 |
| % G2/M | 13 | 5 | 5 | 7 |
| % Apop. | 6 | 20 | 29 | 10 |

Karpas299

| | | | | |
|---|---|---|---|---|
| % G1 | 41 | 71 | 64 | 59 |
| % S | 40 | 7 | 11 | 17 |
| % G2/M | 15 | 17 | 10 | 11 |
| % Apop. | 2 | 5 | 12 | 10 |

Daudi

| | | | | |
|---|---|---|---|---|
| % G1 | 25 | 26 | 24 | 25 |
| % S | 53 | 41 | 53 | 53 |
| % G2/M | 13 | 16 | 12 | 13 |
| % Apop. | 7 | 14 | 8 | 7 |

### 10. In Vivo Efficacy of Chimeric AC10 against Hodgkin's Disease Xenografats

10.1 Materials and Methods

Xenograft models of human Hodgkin's disease: For the disseminated HD model, 1×10⁷ L540cy cells were injected via the tail vein into C.B-17 SCID mice. Treatment with cAC10 was initiated at the indicated times and administered via intraperitoneal injection every four days for a total of 5 injections. Animals were evaluated daily for signs of disseminated disease, in particular hind-limb paralysis. Mice that developed these or other signs of disease were then sacrificed. For the localized model of HD, L540cy cells were implanted with 2×10⁷ cells into the right flank of SCID mice. Therapy with cAC10 was initiated when the tumor size in each group of 5 animals averaged ˜50 mm³. Treatment consisted of intraperitoneal injections of cAC10 every 4 days for 5 injections. Tumor size was determined using the formula (L×W²)/2.

10.2 Results

The in vivo activity of cAC10 was evaluated in SCID mice using L540cy cells. The establishment of human HD models in mice has proven to be difficult. Unlike other HD-derived cell lines that give very poor engraftment in immunodeficient mice, L540cy HD tumor cell models can be successfully established in SCID mice (Kapp et al., 1994, Ann Oncol. 5Suppl 1:121-126). Two separate disease models employing L540cy cells, a disseminated model, and a localized subcutaneous tumor model were used to evaluate the in vivo efficacy of cAC10.

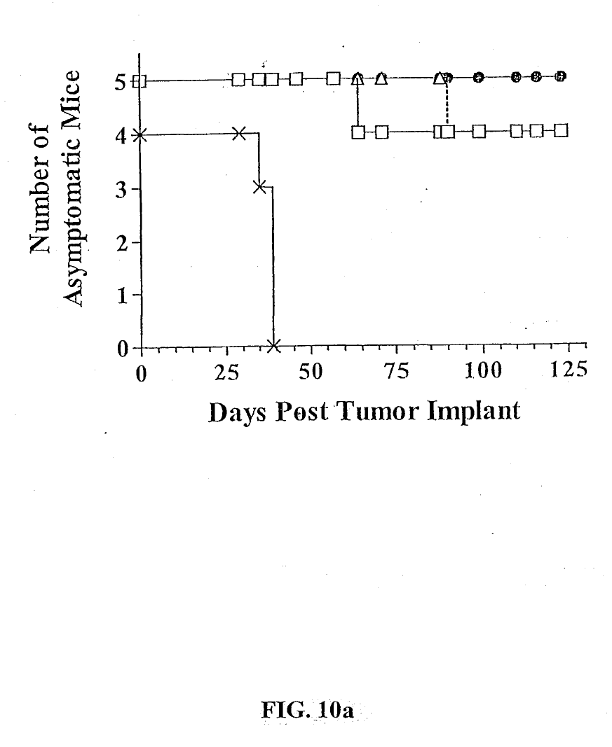

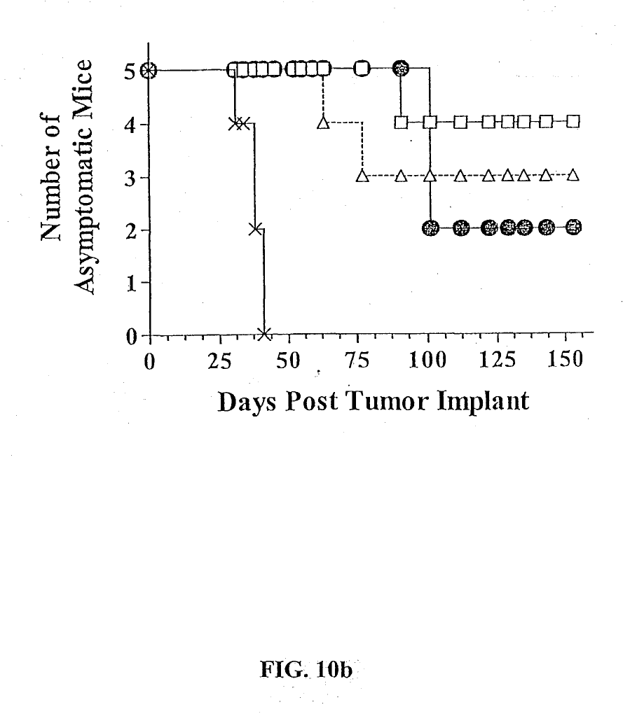

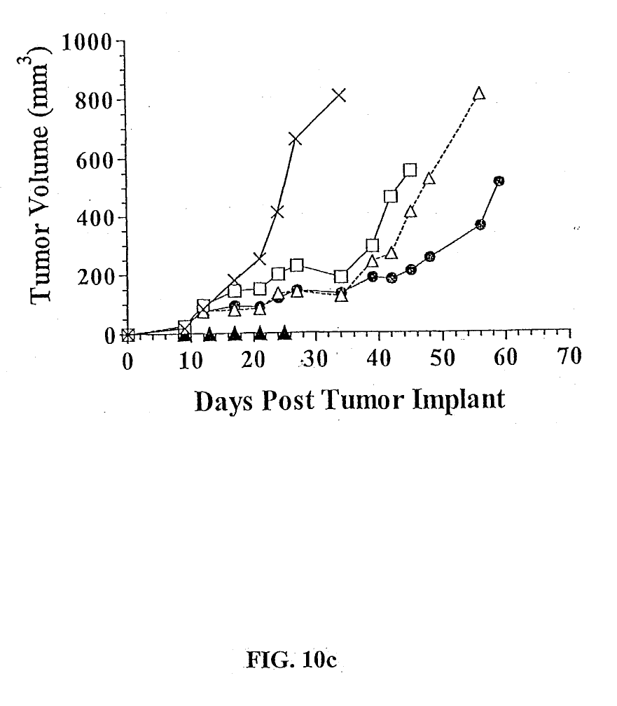

Previous studies have shown that L540cy cells injected intravenously into SCID mice spread in a manner comparable to the dissemination of human HD and show preferential localization to the lymph nodes (Kapp et al., 1994, Ann Oncol. 5Suppl 1:121-126). To evaluate cAC10 in this disseminated HD model, 1×10⁷ L540cy cells were injected via the tail vein into C.B-17 SCID mice. Untreated mice, or those that were treated with a non-binding control mAb, developed signs of disseminated disease, in particular hind-limb paralysis, within 30-40 days of tumor cell injection (FIG. 10A). Mice that developed these or other signs of disease were then sacrificed in accordance with IACUC guidelines. Therapy with cAC10 was initiated one day after tumor cell injection and administered via intraperitoneal injection every four days for a total of 5 injections. All animals (5/5) that received 4 mg/kg/injection dose regimen, and 4/5 that received either 1 mg/kg/injection or 2 mg/kg/injection, survived for greater than 120 days (the length of the study) with no signs of disease.

In a subsequent study the efficacy of cAC10 was further evaluated by varying the day on which therapy was initiated. For this study L540cy cells were injected into SCID mice via the tail vein on day 0 and therapy was initiated either on day 1, day 5, or day 9 (FIG. 10B). In all of the treated groups, cAC10 was administered at 4 mg/kg using a schedule of q4d×5. Consistent with the previous study cAC10 significantly impacted survival of animals that received therapy starting on day 1, with 4/5 animals disease-free after 140 days. When the initiation of therapy was delayed, cAC10 still demonstrated significant efficacy; 3/5 animals that received therapy starting on day 5, and 2/5 starting on day 9, remained disease-free for the length of the study.

cAC10 also demonstrated efficacy in subcutaneous L540cy HD tumor models. SCID mice were implanted with 2×10⁷ cells into the flank. Therapy with cAC10 was initiated when the tumor size in each group of 5 animals averaged 50 mm³. Treatment consisted of intraperitoneal injections of cAC10 every 4 days for S injections using the same doses as in the disseminated model: i.e., 1, 2, and 4 mg/kg/injection. Tumors in the untreated animals grew rapidly and reached an average of >800 mm³ by day 34. cAC10 produced a significant delay in tumor growth at all concentrations tested in a dose dependent manner (FIG. 10C).

### 11. Antitumor Activity of Chimeric Acid Produced in a Hybridoma Cell Line against Subcutaneous L540CY Hodgkin's Disease Xenografts

11.1 Materials and Methods

Chimeric AC10 (cAC10) was generated via homologous recombination essentially as previously described using human IgG1-kappa heavy and light chain conversion vectors (Yarnold and Fell, 1994, Cancer Res. 54: 506-512). These vectors were designed such that the murine immunoglobulin heavy and light chain constant region loci are excised and replaced by the human gamma 1 and kappa constant region loci via homologous recombination. The resulting chimeric hybridoma cell line expresses a chimeric antibody consisting of the heavy and light chain variable regions of the original monoclonal antibody and the human gamma 1 and kappa constant regions.

11.2 Results

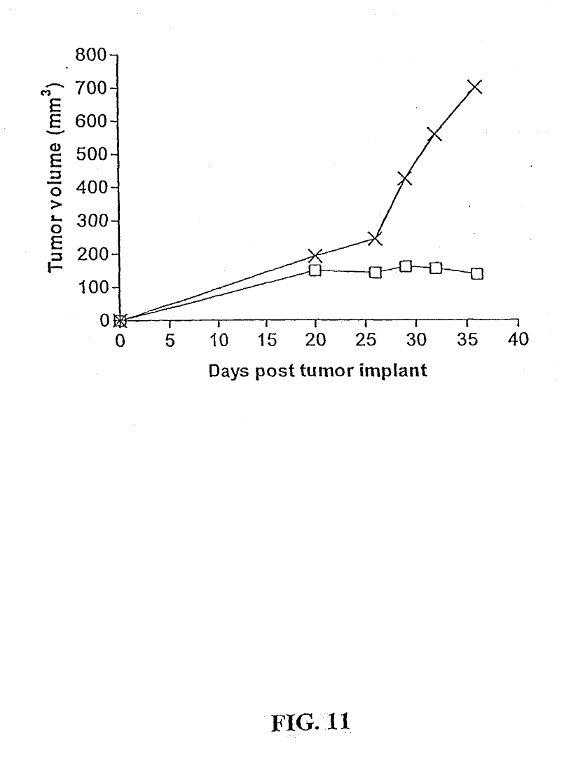

To evaluate the efficacy of cAC10 in vivo, SCID mice were implanted subcutaneously with L540cy cells as described above. When the tumors reached an average size of greater than 150 mm³ the mice were divided into groups that were either untreated or treated with 2 mg/kg cAC10 twice per week for a total of five injections. The tumors in the untreated mice rapidly grew to an average size of greater than 600 mm³ (FIG. 11). In contrast, the average tumor size in the animals treated with cAC10 remained about the same size.

### 12. In Vitro Activity of Chimeric AC10-Drug Conjugates

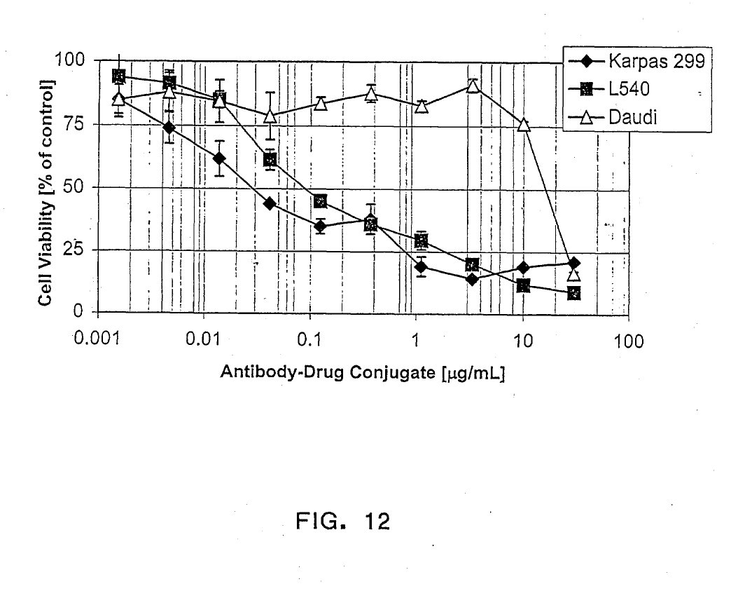

cAC10 can be used to selectively deliver a cytotoxic agent to CD30 positive cells. As shown in FIG. 12, CD30-positive Karpas (ALCL) and L540cy (HD), and the CD30-negative B-cell line Daudi were examined for relative sensitivity to a cytotoxic agent delivered via an cAC10 antibody drug conjugate (ADC). Cells were exposed to cAC10 conjugated to the cytotoxic agent AEB (cAC10-AEB) for 2 h, washed to remove free ADC and cell viability determined at 96 h. Cytotoxicity as determined by the tetrazolium dye (XTT) reduction assay. Both of Karpas 299 and L540cy were sensitive to the cAC10-AEB conjugate with IC₅₀ values (concentration that killed 50% of the cells) of <0.1 microgram/ml. In contrast, the IC50 values on Daudi cells was >10 microgram/ml. All three cell lines were equally sensitive to unconjugated auristatin E by itself (data not shown).

### 13. Antitumor Activity of Chimeric AC10-Drug Conjugates

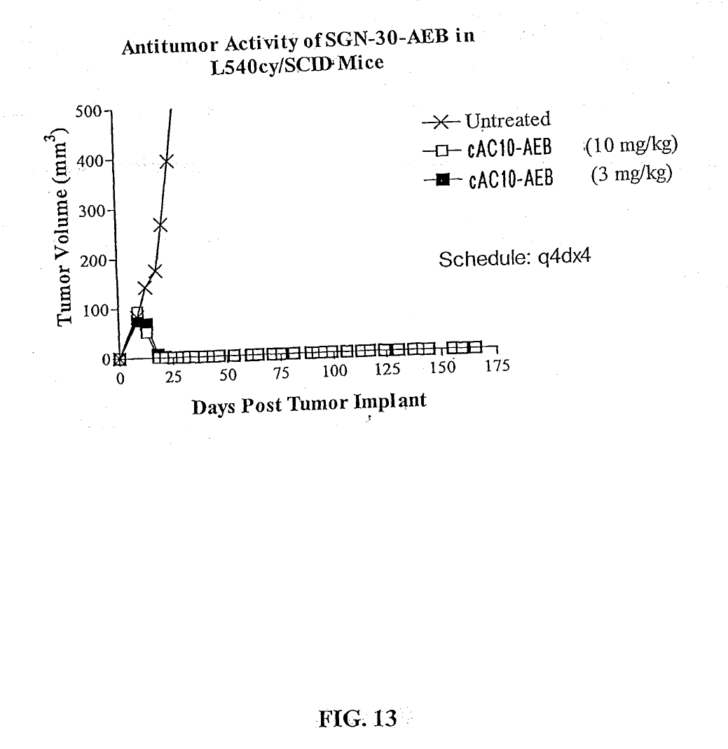

The antitumor activity of chimeric AC10 conjugated to the auristatin E derivative AEB (as described in U.S. application Ser. No. 09/845,786 filed Apr. 30, 2001, which is incorporated by reference here in its entirety) was evaluated in SCID mice bearing L540cy Hodgkin's disease xenografts (FIG. 13). Mice were implanted with L540cy cells subcutaneously and therapy therapy was initiated when the tumors reached an average volume of approximately 75 mm³. Therapy consisted of administering cAC10-AEB at either 3 mg/kg/dose or 10 mg/kg/dose with a total of 4 doses administered at 4-day intervals (q4d×4). The tumors in all of the mice that received cAC10-AEB at both doses completely regressed and were cured by day 18 post tumor implant, 9 days after the start of therapy. These complete regressions remained in effect for the length of the study. These results demonstrate that a chimeric anti-CD30 antibody conjugated to a chemotherapeutic drug, such as auristatin E, can have significant efficacy in Hodgkin's disease.

### 14. Specific Embodiments, Citation of References

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references, including patent applications, patents, and scientific publications, are cited herein, the disclosures of which are incorporated herein by reference in their entireties.

What is claimed is:

1. An antibody that:
(a) immunospecifically binds CD30,
(b) exerts a cytostatic or cytotoxic effect on a Hodgkin's Disease cell line, which cytostatic or cytotoxic effect is complement-independent and achieved in the absence of:
(i) conjugation to a cytostatic or cytotoxic agent, and
(ii) effector cells, and
(c) is not monoclonal antibody AC10 or HeFi-1 and does not result from cleavage of AC10 or HeFi-1 with papain or pepsin.

2. The antibody of claim 1, which comprises a human constant domain.

3. An antibody that:
(a) competes for binding to CD30 with monoclonal antibody AC10 or HeFi-1,
(b) exerts a cytostatic or cytotoxic effect on a Hodgkin's Disease cell line, which cytostatic or cytotoxic effect is not complement-dependent and is achieved in the absence of:
(i) conjugation to a cytostatic or cytotoxic agent, and
(ii) effector cells, and
(c) is not monoclonal antibody AC10 or HeFi-1 and does not result from cleavage of AC10 or HeFi-1 with papain or pepsin.

4. The antibody of claim 3, which comprises a human constant domain.

5. An antibody that:
(a) immunospecifically binds CD30;
(b) exerts a cytostatic or cytotoxic effect on a Hodgkin's Disease cell line, wherein said antibody exerts the cytostatic or cytotoxic effect on the Hodgkin's Disease cell line in the absence of conjugation to a cytostatic or cytotoxic agent, respectively; and
(c) is not monoclonal antibody AC10 or HeFi-1 and does not result from cleavage of AC10 or HeFi-1 with papain or pepsin, wherein the cytostatic or cytotoxic effect is exhibited upon performing a method comprising:
(i) immobilizing said antibody in a well, said well having a culture area of about 0.33 cm²;
(ii) adding 5,000 cells of the Hodgkin's Disease cell line in the presence of only RPMI with 10% fetal bovine serum or 20% fetal bovine serum to the well;
(iii) culturing the cells in presence of only said antibody and RPMI with 10% fetal bovine serum or 20% fetal bovine serum for a period of 72 hours to form a Hodgkin's Disease cell culture;
(iv) exposing the Hodgkin's Disease cell culture to 0.5 μCi/well of ³H-thymidine during the final 8 hours of said 72-hour period; and
(v) measuring the incorporation of ³H-thymidine into cells of the Hodgkin's Disease cell culture,
wherein the antibody has a cytostatic or cytotoxic effect on the Hodgkin's Disease cell line if the cells of the Hodgkin's Disease cell culture have reduced ³H-thymidine incorporation compared to cells of the same Hodgkin's Disease cell line cultured under the same conditions but not contacted with the antibody.

6. The antibody of claim 5, which comprises a human constant domain.

7. The antibody of any one of claims 1-6 which is purified.

8. The antibody of any one of claims 1-6 which is a human, humanized or chimeric antibody.

9. The antibody of any one of claims 1-6 which is conjugated to a cytotoxic agent.

10. The antibody of claim 8 which is conjugated to a cytotoxic agent.

11. The antibody of any one of claims 1-6 which is a fusion protein comprising the amino acid sequence of a second protein that is not an antibody.

12. The antibody of any one of claims 1-4, wherein the cytostatic or cytotoxic effect is exhibited upon performing a method comprising:
(a) immobilizing said antibody in a well, said well having a culture area of about 0.33 cm²;
(b) adding 5,000 cells of the Hodgkin's Disease cell line in the presence of only RPMI with 10% fetal bovine serum or 20% fetal bovine serum to the well;
(c) culturing the cells in presence of only said antibody and RPMI with 10% fetal bovine serum or 20% fetal bovine serum for a period of 72 hours to form a Hodgkin's Disease cell culture;
(d) exposing the Hodgkin's Disease cell culture to 0.5 μCi/well of ³H-thymidine during the final 8 hours of said 72-hour period; and
(e) measuring the incorporation of ³H-thymidine into cells of the Hodgkin's Disease cell culture,
wherein the antibody has a cytostatic or cytotoxic effect on the Hodgkin's Disease cell line if the cells of the Hodgkin's Disease cell culture have reduced ³H-thymidine incorporation compared to cells of the same Hodgkin's Disease cell line cultured under the same conditions but not contacted with the antibody.

13. The antibody of claim 5, wherein the said ³H-thymidine incorporation is reduced by at least 15%.

14. The antibody of claim 12, wherein the said ³H-thymidine incorporation is reduced by at least 15%.

15. A pharmaceutical composition comprising a therapeutically effective amount of the antibody of any one of claims 1-6 and a pharmaceutically acceptable carrier.

16. A method for the treatment of Hodgkin's Disease in a subject comprising administering to the subject, in an amount effective for said treatment, the antibody of any one of claims 1-6.

17. A method for the treatment of Hodgkin's Disease in a subject comprising administering to the subject, in an amount effective for said treatment, the pharmaceutical composition of claim 15.

18. An isolated nucleic acid comprising a nucleotide sequence encoding a heavy chain of the antibody of any one of claims 1-6.


SEQUENCE LISTING (32 sequences)

SEQ ID NO: 1 - DNA, 351 bp, Mus musculus
  CDS (1)...(351)
cagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgca

SEQ ID NO: 2 - protein, 117 aa, Mus musculus
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 3 - DNA, 15 bp, Mus musculus
gactactatataacc

SEQ ID NO: 4 - protein, 5 aa, Mus musculus
DYYIT

SEQ ID NO: 5 - DNA, 51 bp, Mus musculus
tggatttatcctggaagcggtaatactaagtacaatgagaagttcaagggc

SEQ ID NO: 6 - protein, 17 aa, Mus musculus
WIYPGSGNTKYNEKFKG

SEQ ID NO: 7 - DNA, 24 bp, Mus musculus
tatggtaactactggtttgcttac

SEQ ID NO: 8 - protein, 8 aa, Mus musculus
YGNYWFAY

SEQ ID NO: 9 - DNA, 333 bp, Mus musculus
  CDS (1)...(333)
gacattgtgctgacccaatctccagcttctttggctgtgtctctagggcagagggccaccatctcctgcaaggccagccaaagtgttgattttgatggtgatagttatatgaactggtaccaacagaaaccaggacagccacccaaagtcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 10 - protein, 111 aa, Mus musculus
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 11 - DNA, 45 bp, Mus musculus
aaggccagccaaagtgttgattttgatggtgatagttatatgaac

SEQ ID NO: 12 - protein, 15 aa, Mus musculus
KASQSVDFDGDSYMN

SEQ ID NO: 13 - DNA, 21 bp, Mus musculus
gctgcatccaatctagaatct

SEQ ID NO: 14 - protein, 7 aa, Mus musculus
AASNLES

SEQ ID NO: 15 - DNA, 27 bp, Mus musculus
cagcaaagtaatgaggatccgtggacg

SEQ ID NO: 16 - protein, 9 aa, Mus musculus
QQSNEDPWT

SEQ ID NO: 17 - DNA, 375 bp, Mus musculus
  CDS (1)...(375)
gaggtgaagctggtggagtctggaggaggcttggtacagcctgggggttctctgagactctcctgtgcaacttctgggttcaccttcagtgattactatatgaactgggtccgccagcctccaggaaaggctcttgagtggttgggttttattagaaacaaagctaatggttacacaacagagttcagtgcatctgtgatgggtcggttcaccatctccagagatgattcccaaagcatcctctatcttcagatgaacaccctgagagctgaggacagtgccacttattactgtgcaagagatcccccctatggtaacccccattattatgctatggactactggggtcaaggaacctcagtcaccgtctcctca

SEQ ID NO: 18 - protein, 125 aa, Mus musculus
EVKLVESGGGLVQPGGSLRLSCATSGFTFSDYYMNWVRQPPGKALEWLGFIRNKANGYTTEFSASVMGRFTISRDDSQSILYLQMNTLRAEDSATYYCARDPPYGNPHYYAMDYWGQGTSVTVSS

SEQ ID NO: 19 - DNA, 15 bp, Mus musculus
gattactatatgaac

SEQ ID NO: 20 - protein, 5 aa, Mus musculus
DYYMN

SEQ ID NO: 21 - DNA, 57 bp, Mus musculus
tttattagaaacaaagctaatggttacacaacagagttcagtgcatctgtgatgggt

SEQ ID NO: 22 - protein, 19 aa, Mus musculus
FIRNKANGYTTEFSASVMG

SEQ ID NO: 23 - DNA, 42 bp, Mus musculus
gatcccccctatggtaacccccattattatgctatggactac

SEQ ID NO: 24 - protein, 14 aa, Mus musculus
DPPYGNPHYYAMDY

SEQ ID NO: 25 - DNA, 333 bp, Mus musculus
  CDS (1)...(333)
gacattgtgctgacccagtctcctgcttccttagctgtttctctggggcagagggccaccatctcatgcagggccagcaaaagtgtcagtgcatctggctataattatatgcactggtaccaacagaaagcagggcagccacccaaactcctcatccatcttgcatccaacctagaatctggggtccctgccaggttcagtggcagtgggtctgggacagacttcaccctcaacatccatcctgtggaggaggaggatgcttcaacctattactgtcagcacagtggggagcttccattcacgttcggctcggggacaaagttggaaataaaa

SEQ ID NO: 26 - protein, 111 aa, Mus musculus
DIVLTQSPASLAVSLGQRATISCRASKSVSASGYNYMHWYQQKAGQPPKLLIHLASNLESGVPARFSGSGSGTDFTLNIHPVEEEDASTYYCQHSGELPFTFGSGTKLEIK

SEQ ID NO: 27 - DNA, 45 bp, Mus musculus
agggccagcaaaagtgtcagtgcatctggctataattatatgcac

SEQ ID NO: 28 - protein, 15 aa, Mus musculus
RASKSVSASGYNYMH

SEQ ID NO: 29 - DNA, 21 bp, Mus musculus
cttgcatccaacctagaatct

SEQ ID NO: 30 - protein, 7 aa, Mus musculus
LASNLES

SEQ ID NO: 31 - DNA, 27 bp, Mus musculus
cagcacagtggggagcttccattcacg

SEQ ID NO: 32 - protein, 9 aa, Mus musculus
QHSGELPFT